SEQUENCE LISTING (103 sequences)

SEQ ID NO: 1 - DNA, 3069 bp, unidentified
  source 1..3069: mol_type = unassigned DNA; note = Triticum aestivum L.
tagaaactttctgaatcacacgtccagttttatccgcatcgagagacgcaccacatccgggttattcaggaaaattgctgctaggaatttgatgactggatttctgttcgttaaccagaaatgcgaaagtaacctcggagccatcttgagaatttgccatcagttgccgcttatcttgcgaactgttgcttagaagcagcgcctccgaactgaacctcattttagagcggaaagctgaacccttttgcggttcatgaggtgcaaaagtatactacggagtagtttccatccatgttcttgcaaattttcttggcttggatacagttgcagtagacgaagatttgtaaccgcagcacattattccatgaaaaatgctcaaatgttgtgtcctccaaaagcagaggttcaggataactgacagtcgtcttggccggtgccttcttaccgaaggcgaaggctggctccatcacttgggccattgaattgccttttgagctcatcgcaccggccgataacaaactccggccgacatatccactggcccaaaggagcattcaggacgagcacgcggcaaaatgatttgcaagttgcaatcccggcatgctgcagcacagtataaatacctggccacacacaccagctgaatccatcagttctccaccgtcctcttgcagagcacacacagagctgaagacgatggcgaacaaacacttgtccctctcgctcttcctcgtcctccttggcctgtcggccagcttggcctccgggcaagtcctgtttcaggtaagctcctgttcgtcggcaagaactgtcttcagactgtgatcatgttttcttttggactgaggcctcctttggtttgtaggaatctcatatgaatttcatagaataggatttttaaatgaaaaattcctatggagcccttcagtttgtacgaataaatttctattactgtgtatgatagggttcaatccttcacatttcaaagaaaaaaaatattaatctagactcaatgaaaaaattcctatcttatgcattaaatgacatctctttctttatagaaattcagatgcatgtcatctcacttcatatgaagttcctattcctataatattcctatcctatgaaccaaagagggcctcaactttgagttgtgcgtgcggcagggtttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcacggcggagcacaaggacggccggggcatctactgcatcttcgaaggcggcacccccgacgcccgcctcgactggggcccccacatgatctgccgtgacgaccggccctacgccgacggcaccggcaacccggacaccggcgccgacttcggggccgcccccgacatcgaccacctcaacccgcgcgtccagaaggagctcgtcgagtggctcaactggctcaagaccgacatcggcttcgacggatggcgcttcgacttcgccaagggctactccgcggacgtcgccaagatttacatcgaccgctcggagcccggcttcgccgtggccgagatatggacgtcgctggcgtacggcggtgacggcaagcccaacctgaaccaggaccagcaccggcaggagctggtgaactgggtggacaaggtcggcggcaaaggccccgctaccacgttcgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggatccacgcagcacatgtggcccttcccttctgacagggtcatgcagggatatgcctacatcctcacgcacccagggaccccatgcatcgtgagtcatcgtaccaatactacatcatatctaaattttcttctcgttttttcgttcattatagaaaaccacgaccgaactgatgaaaataatcgtgattctccagttctacgatcatttcttcgattggggccttaaggaggagatcgatcgcctggtgtcaatcaggacccggcatgggatacacagtgagagcaagctgcaaatcatagaggccgacgccgacctttatctcgccgagatcgacggcaaggtcatcgtcaagctcgggccaagatacgatgtggggcacctcattccgggaggcttcaaggtgggcgcgcacggcaatgactatgccgtatgggagaaaatatgagcaaaattaccaaagcagctctacgagttttttacaatatgaatgacatacatattagtccgagctcacgttgtccacatagtacgatttagtacttcctccatgtaaaagtgaggatgagggacattcattgtatttttgataaaataataatcaataaggatttttgcactcttggcttacttagtgttggttgaatgaaaactgagcagccacgacctcgtacgggagcataccggagtagtggtagactcacgagcgttttgttacggaattgtgttacgggggttgggtttaaaggagatagtactaatgcttatttggtgtatatggcaattacgtaatgatcttactcatgataaaaaggttcctccagtggatattactgttgactttttagtgagctatatgaattttatttgtgatgcgaagaagttgagcactgaggaaatactcaaagggaaaatgccggttgctgattgggctttgcccgaagtgagaattaatcctgctcctaagccttggccacctcctactacaaactttgttgctctattagttgatggatcttttacaattgattctggcgccgcaaccata

SEQ ID NO: 2 - DNA, 1380 bp, unidentified
  source 1..1380: mol_type = unassigned DNA; note = Triticum aestivum L.
atgctgcagcacagtataaatacctggccacacacaccagctgaatccatcagttctccaccgtcctcttgcagagcacacacagagctgaagacgatggcgaacaaacacttgtccctctcgctcttcctcgtcctccttggcctgtcggccagcttggcctccgggcaagtcctgtttcagggtttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcacggcggagcacaaggacggccggggcatctactgcatcttcgaaggcggcacccccgacgcccgcctcgactggggcccccacatgatctgccgtgacgaccggccctacgccgacggcaccggcaacccggacaccggcgccgacttcggggccgcccccgacatcgaccacctcaacccgcgcgtccagaaggagctcgtcgagtggctcaactggctcaagaccgacatcggcttcgacggatggcgcttcgacttcgccaagggctactccgcggacgtcgccaagatttacatcgaccgctcggagcccggcttcgccgtggccgagatatggacgtcgctggcgtacggcggtgacggcaagcccaacctgaaccaggaccagcaccggcaggagctggtgaactgggtggacaaggtcggcggcaaaggccccgctaccacgttcgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggatccacgcagcacatgtggcccttcccttctgacagggtcatgcagggatatgcctacatcctcacgcacccagggaccccatgcatcttctacgatcatttcttcgattggggccttaaggaggagatcgatcgcctggtgtcaatcaggacccggcatgggatacacagtgagagcaagctgcaaatcatagaggccgacgccgacctttatctcgccgagatcgacggcaaggtcatcgtcaagctcgggccaagatacgatgtggggcacctcattccgggaggcttcaaggtgggcgcgcacggcaatgactatgccgtatgggagaaaatatga

SEQ ID NO: 3 - DNA, 1380 bp, unidentified; identical to SEQ ID NO: 2
  source 1..1380: mol_type = unassigned DNA; note = Triticum aestivum L.

SEQ ID NO: 4 - protein, 459 aa, unidentified
  source 1..459: note = Triticum aestivum L.
MLQHSINTWPHTPAESISSPPSSCRAHTELKTMANKHLSLSLFLVLLGLSASLASGQVLFQGFNWESWKHNGGWYNFLMGKVDDIAAAGITHVWLPPASQSVAEQGYMPGRLYDLDASKYGNKAQLKSLIGALHGKGVKAIADIVINHRTAEHKDGRGIYCIFEGGTPDARLDWGPHMICRDDRPYADGTGNPDTGADFGAAPDIDHLNPRVQKELVEWLNWLKTDIGFDGWRFDFAKGYSADVAKIYIDRSEPGFAVAEIWTSLAYGGDGKPNLNQDQHRQELVNWVDKVGGKGPATTFDFTTKGILNVAVEGELWRLRGTDGKAPGMIGWWPAKAVTFVDNHDTGSTQHMWPFPSDRVMQGYAYILTHPGTPCIFYDHFFDWGLKEEIDRLVSIRTRHGIHSESKLQIIEADADLYLAEIDGKVIVKLGPRYDVGHLIPGGFKVGAHGNDYAVWEKI

SEQ ID NO: 5 - DNA, 2888 bp, unidentified
  source 1..2888: mol_type = unassigned DNA; note = Triticum aestivum L.
tttatgtccagttttatccgcctcgagggacgcacctcatccgggttatccaggaatttgctcacccggattttagctggccgtcgtcaacctgaaacgcgcaagtaaccgcggagccatctcgccaaagttaccgtcagttgcccctgatcttacgagctgttatagcacagctgaccctcattgcagtgcgcgagagccgaaacctattcgattgataagctgcaaaaccttctcgacttttatcacagttattgaattagtagacgatggctcgtaagcacaaagactattccatttccaccgctagtttggcgtgtgagttctgaggaattctcaagttcagcgggataactgacagccgtattggccggtaacaaacttctcatacaagggtggctccatcactcgccttttgagctcaccgcaccggccgataacaaactccggccgacatatccatcgagccgccggcccaacggagcattgaagtcgagcacgtacaccggaacatgtcttgcaagttgcccaccggcatgctgcagcctgcagcacactataaatacctggccagacgcaccagctgaatccatcaggtctccatcctcctcttcgagagcacagctagatagagctgaagaacatggcgagcaaacactcgtccctctccctcttcctcgtcctccttggcctgtcggccagcttggcctccgggcaagtcctgtttcaggtaagatcgtgccctgtcttcagcttctacacgtactactgtgatcatgtttcggggactgagctttgagttctgctgcgtgcggcagggtttcaactgggagtcgtggaagcacaatggcgggtggtacaacttcctgatgggcaaggtggacgacatcgccgccgccggcgtcacgcacgtctggctccctccggcgtcgcagtccgtcgccgagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacgggcggggcatctactgcatcttcgagggcggcaccccggacgcgcgcctcgactggggcccccacatgatctgccgcgacgatcggccctacgccgacggcaccggcaacccagacacgggcgccgacttcggggccgccccggacatcgaccacctcaacccgcgtgtgcagaaggagctcgtcgagtggctcaactggctcaggaccaacgtcggcttcgacggctggcgcttcgacttcgccaagggctactccgcggacgtcgccaagatctacatcgaccgctccggggccagcttcgccgtggccgagatatggacgtcgctggcgtacggtggggacggcaagccgaacctcaaccaggacccgcaccggcaggagctggtgaactgggtgaacaaggtgggcggctccggccccggcaccacgttcgacttcaccaccaagggcatcctcaacgtggccgtggagggcgagctctggcggctgcgcggcaccgacggcaaggcgccaggcatgatcgggtggtggccagccaaggcggtgaccttcgtggacaaccacgacaccggctccacgcagcacatgtggcccttcccttctgacagggtcatgcagggatacgcctacatcctcacgcaccctgggaccccatgcatcgtgagtcatcctaccagttcatcgtctaaattgctcttcttttttcgttcatataagaacccatgcatgagtgacgaaaaatattcgtgattcttcagttctacgatcatttcttcgactggggcctgaaggaggagatcgatcgcctggtgtcaatcaggacccggcaggggatacacagtgagagcaagctgcaaatcatagaggctgacgccgacctttacctggctgagatcgacggcaaggtcatcgtcaagctcgggccaagatacgatgtcgggcacctcattccccaaggcttcaaggtggtcgcgcacggcaatgactatgccgtatgggagaaaatataagcaaaattatccaagcagctccacaattttttttcctatatatgaatgccacacctattagtccgagctcgtgttgtccacatagtacgattttagtacttcctccatgtaaaagtgaggatgagggacatccattgtatttttcataaataataatcaataatcgttttcgctactcatggtttactcaatgttcattgaccaaaaattgcatagagcacgaccatgtgggagcgtgaaacctgaaacttgaggctttgatggtaggagctctggctttgcattaaggattagggctcttctaggatcagataccgaccagaaggggagggggtgaatgggaacttcaacaatttcttcaaaaaaaatgagttttttgagcaaaaactaagtaaatagaacaagtgcagattagaatagagaataaactaaactgaaaagagaaataattgaggttaatctaagtagcaattccaaactaaacatgacataggtagcagaataacaagtgctcaaaatcgtcttcgaagcaatcacaacaatcaccgtgaaataagacggggagaagttcttcgaacgcatgtacgttacataagtaaatgagtttgataaagtaaaaaacatgtaagcttgacgaaggcatagatttgttcgaccagtccgagttgctggcacaacactatgatctgtttggaggggccgagattaagctcggaagacaacgtcttcatcctatgttccttcagataaagtcacttagacttcgctcaattac

SEQ ID NO: 6 - DNA, 1502 bp, unidentified
  source 1..1502: mol_type = unassigned DNA; note = Triticum aestivum L.
ccatcctcctcttcgagagcacagctagatagagctgaagaacatggcgagcaaacactcgtccctctccctcttcctcgtcctccttggcctgtcggccagcttggcctccgggcaagtcctgtttcagggtttcaactgggagtcgtggaagcacaatggcgggtggtacaacttcctgatgggcaaggtggacgacatcgccgccgccggcgtcacgcacgtctggctccctccggcgtcgcagtccgtcgccgagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacgggcggggcatctactgcatcttcgagggcggcaccccggacgcgcgcctcgactggggcccccacatgatctgccgcgacgatcggccctacgccgacggcaccggcaacccagacacgggcgccgacttcggggccgccccggacatcgaccacctcaacccgcgtgtgcagaaggagctcgtcgagtggctcaactggctcaggaccaacgtcggcttcgacggctggcgcttcgacttcgccaagggctactccgcggacgtcgccaagatctacatcgaccgctccggggccagcttcgccgtggccgagatatggacgtcgctggcgtacggtggggacggcaagccgaacctcaaccaggacccgcaccggcaggagctggtgaactgggtgaacaaggtgggcggctccggccccggcaccacgttcgacttcaccaccaagggcatcctcaacgtggccgtggagggcgagctctggcggctgcgcggcaccgacggcaaggcgccaggcatgatcgggtggtggccagccaaggcggtgaccttcgtggacaaccacgacaccggctccacgcagcacatgtggcccttcccttctgacagggtcatgcagggatacgcctacatcctcacgcaccctgggaccccatgcatcttctacgatcatttcttcgactggggcctgaaggaggagatcgatcgcctggtgtcaatcaggacccggcaggggatacacagtgagagcaagctgcaaatcatagaggctgacgccgacctttacctggctgagatcgacggcaaggtcatcgtcaagctcgggccaagatacgatgtcgggcacctcattccccaaggcttcaaggtggtcgcgcacggcaatgactatgccgtatgggagaaaatataagcaaaattatccaagcagctccacaattttttttcctatatatgaatgccacacctattagtccgagctcgtgttgtccacatagtacgattttagtacttcctccatgtaaaagtgaggatgagggacatccattgtatttttcataaataataatcaataatcgttttcgcta

SEQ ID NO: 7 - DNA, 1284 bp, unidentified
  source 1..1284: mol_type = unassigned DNA; note = Triticum aestivum L.
atggcgagcaaacactcgtccctctccctcttcctcgtcctccttggcctgtcggccagcttggcctccgggcaagtcctgtttcagggtttcaactgggagtcgtggaagcacaatggcgggtggtacaacttcctgatgggcaaggtggacgacatcgccgccgccggcgtcacgcacgtctggctccctccggcgtcgcagtccgtcgccgagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacgggcggggcatctactgcatcttcgagggcggcaccccggacgcgcgcctcgactggggcccccacatgatctgccgcgacgatcggccctacgccgacggcaccggcaacccagacacgggcgccgacttcggggccgccccggacatcgaccacctcaacccgcgtgtgcagaaggagctcgtcgagtggctcaactggctcaggaccaacgtcggcttcgacggctggcgcttcgacttcgccaagggctactccgcggacgtcgccaagatctacatcgaccgctccggggccagcttcgccgtggccgagatatggacgtcgctggcgtacggtggggacggcaagccgaacctcaaccaggacccgcaccggcaggagctggtgaactgggtgaacaaggtgggcggctccggccccggcaccacgttcgacttcaccaccaagggcatcctcaacgtggccgtggagggcgagctctggcggctgcgcggcaccgacggcaaggcgccaggcatgatcgggtggtggccagccaaggcggtgaccttcgtggacaaccacgacaccggctccacgcagcacatgtggcccttcccttctgacagggtcatgcagggatacgcctacatcctcacgcaccctgggaccccatgcatcttctacgatcatttcttcgactggggcctgaaggaggagatcgatcgcctggtgtcaatcaggacccggcaggggatacacagtgagagcaagctgcaaatcatagaggctgacgccgacctttacctggctgagatcgacggcaaggtcatcgtcaagctcgggccaagatacgatgtcgggcacctcattccccaaggcttcaaggtggtcgcgcacggcaatgactatgccgtatgggagaaaatataa

SEQ ID NO: 8 - protein, 427 aa, unidentified
  source 1..427: note = Triticum aestivum L.
MASKHSSLSLFLVLLGLSASLASGQVLFQGFNWESWKHNGGWYNFLMGKVDDIAAAGVTHVWLPPASQSVAEQGYMPGRLYDLDASKYGNKAQLKSLIGALHGKGVKAIADIVINHRTAERKDGRGIYCIFEGGTPDARLDWGPHMICRDDRPYADGTGNPDTGADFGAAPDIDHLNPRVQKELVEWLNWLRTNVGFDGWRFDFAKGYSADVAKIYIDRSGASFAVAEIWTSLAYGGDGKPNLNQDPHRQELVNWVNKVGGSGPGTTFDFTTKGILNVAVEGELWRLRGTDGKAPGMIGWWPAKAVTFVDNHDTGSTQHMWPFPSDRVMQGYAYILTHPGTPCIFYDHFFDWGLKEEIDRLVSIRTRQGIHSESKLQIIEADADLYLAEIDGKVIVKLGPRYDVGHLIPQGFKVVAHGNDYAVWEKI

SEQ ID NO: 9 - DNA, 2833 bp, unidentified
  source 1..2833: mol_type = unassigned DNA; note = Triticum aestivum L.
attcaggaaattttgccaggaatttcctgacccggagggagtacttctcaactgacccccatctcactgcgcgaaagctgaaatcttttcggttgcaaaaccttctcaattttcttcgcctgatttacactgttttagtagatgatggttggtaacccgtaacacacacacacacacacacacacacacaattccaattccaccgttagaaaggacactataaatacctgagttctgaggaattctgaagatgaaggacactataaattccaccgttagtgcactaaagatgaaggacactgtttatttccccctagtttggtgtgtgagttctgaggaattctcaagttcagtaggataactgacagccgtattgggtgccgtctcatacaagggtggctccatcactcgccttttgagctcaccgcaccggccgataacaaactccggccgacatatccatcgatccaacggagcattgaagtcgatcgcaccggaacatgtactctttgcaagatgcccaccggcatcctgcagcctgcagcacactataaatacctggccagacgcaccagctgaatccatcagttctccatcgtcctcttccaaagcacagctagctagagctcaaggtcatggcgaacaaacactcgtccctctccctcttcctcgtcctccttggcctgtcggcaagcttagcctccggtcaagtcctgtttcaggtaagatcatgtcctgtcttcaacttctacacgtactgtgatcatgtttcggggactgagttttcagttctgcatgcggcagggtttcaactgggagtcgtggaagcacaatggcgggtggtacaacttcctgatgggcaaggtggacgacatcgccgccgccggcgtcacgcacgtgtggctccccccggcgtcgcagtccgtcgccgagcaagggtacatgccgggccggctgtacgacctggacgcgtccaagtacggcaacaaggcgcagctcaagtccctgatcggggcgctccacggcaagggcgtcaaggccatcgccgacatcgtcatcaaccaccgcacggcggagcgcaaggacggccggggcatctactgcatcttcgagggcggcacccctgacgcgcgcctcgactggggcccccacatgatctgccgcgacgaccggccctacgccgacggcacgggcaacccagacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctcggagcccagcttcgccgtggccgagatatggacgtcgctggcgtacggcggggacggcaagcccaacctcaaccaggaccagcaccggcaggagctggtgaactgggtgaacaaggtgggcggctccggccccggcaccacgttcgacttcaccaccaaaggcatcctcaacgtggctgtggagggcgagctgtggcggctgcgcggcaccgacggcaaggcgccaggcatgatcgggtggtggccagccaaggcggtgaccttcgtggacaaccatgacaccggctccacgcagcacatgtggcccttcccttccgacaaggtcatgcagggatacgcctacatcctcacgcaccctgggaccccatgcatcgtgagtcatcgtgccattgccaaatcatcatatctaaattgtcttttttttcgtccgttcataagaaaccatgacctaacttgacaaaaaaaatgatattcttcagttctacgatcatttcttcgactgggggctgaaggaggagatcgatcgcctggtgtcaatcaggacccggcaggggatacacagtgagagcaagctgcaaatcatagaggccgacgccgacctttacctggccgagatcgatggcaaggtcatcgtcaagctcgggccaagatacgatgtcgggcacctcattcctcaaggcttcaaggtggtcgcgcacggcaatgactatgccgtatgggagaaaatataagcaaaattatccgagctgctccacaatttttcttccctatatatgaatgtcacacctattagtccgagctcgtgttgtccacatagtacgattttagtacttcctccatgtaaaagtgaggatgagggacatccattgtatttttcataaataataatcaataagtatttttgctactcatggtttactcgatgttggttgaccacaaattatgcagcgcacgaccactgactcgtgcgagagcatgcaatctccaagcttgatgcttggggtggtaggagctctgcctttgcattaaggattagagctctacataagaaagcatggaaaggagaagaagaaatcttcacaaggcgatgtctgtagcttagttatacaatgctcagctatcagttctgaattaactttgatcagagctgccatgtgttgtagaaaatctgactatttctctttttttgcaaaaaaaaaaaaaaaacccgacggtttctttcattcaaaatatgtactgagcaaagtatgatggaactgaagctgttcatctgagttgcaccaaagtgtggcatgttgcagctaagtttgaacgatgtggcgaacggatagcggtggattcgtgggctgcaaattctagtagctggtcacaaagaaagcaaacatggttgtaatcgcagcctcctgcatgcggtctgtcagcggtgtgaagctgattttgttttttttttcgaaaag

SEQ ID NO: 10 - DNA, 1445 bp, unidentified
  source 1..1445: mol_type = unassigned DNA; note = Triticum aestivum L.
ctcttccaaagcacagctagctagagctcaaggtcatggcgaacaaacactcgtccctctccctcttcctcgtcctccttggcctgtcggcaagcttagcctccggtcaagtcctgtttcagggtttcaactgggagtcgtggaagcacaatggcgggtggtacaacttcctgatgggcaaggtggacgacatcgccgccgccggcgtcacgcacgtgtggctccccccggcgtcgcagtccgtcgccgagcaagggtacatgccgggccggctgtacgacctggacgcgtccaagtacggcaacaaggcgcagctcaagtccctgatcggggcgctccacggcaagggcgtcaaggccatcgccgacatcgtcatcaaccaccgcacggcggagcgcaaggacggccggggcatctactgcatcttcgagggcggcacccctgacgcgcgcctcgactggggcccccacatgatctgccgcgacgaccggccctacgccgacggcacgggcaacccagacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctcggagcccagcttcgccgtggccgagatatggacgtcgctggcgtacggcggggacggcaagcccaacctcaaccaggaccagcaccggcaggagctggtgaactgggtgaacaaggtgggcggctccggccccggcaccacgttcgacttcaccaccaaaggcatcctcaacgtggctgtggagggcgagctgtggcggctgcgcggcaccgacggcaaggcgccaggcatgatcgggtggtggccagccaaggcggtgaccttcgtggacaaccatgacaccggctccacgcagcacatgtggcccttcccttccgacaaggtcatgcagggatacgcctacatcctcacgcaccctgggaccccatgcatcttctacgatcatttcttcgactgggggctgaaggaggagatcgatcgcctggtgtcaatcaggacccggcaggggatacacagtgagagcaagctgcaaatcatagaggccgacgccgacctttacctggccgagatcgatggcaaggtcatcgtcaagctcgggccaagatacgatgtcgggcacctcattcctcaaggcttcaaggtggtcgcgcacggcaatgactatgccgtatgggagaaaatataagcaaaattatccgagctgctccacaatttttcttccctatatatgaatgtcacacctattagtccgagctcgtgttgtccacatagtacgattttagtacttcctccatgtaaaagtgaggatgag

SEQ ID NO: 11 - DNA, 1284 bp, unidentified
  source 1..1284: mol_type = unassigned DNA; note = Triticum aestivum L.
atggcgaacaaacactcgtccctctccctcttcctcgtcctccttggcctgtcggcaagcttagcctccggtcaagtcctgtttcagggtttcaactgggagtcgtggaagcacaatggcgggtggtacaacttcctgatgggcaaggtggacgacatcgccgccgccggcgtcacgcacgtgtggctccccccggcgtcgcagtccgtcgccgagcaagggtacatgccgggccggctgtacgacctggacgcgtccaagtacggcaacaaggcgcagctcaagtccctgatcggggcgctccacggcaagggcgtcaaggccatcgccgacatcgtcatcaaccaccgcacggcggagcgcaaggacggccggggcatctactgcatcttcgagggcggcacccctgacgcgcgcctcgactggggcccccacatgatctgccgcgacgaccggccctacgccgacggcacgggcaacccagacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctcggagcccagcttcgccgtggccgagatatggacgtcgctggcgtacggcggggacggcaagcccaacctcaaccaggaccagcaccggcaggagctggtgaactgggtgaacaaggtgggcggctccggccccggcaccacgttcgacttcaccaccaaaggcatcctcaacgtggctgtggagggcgagctgtggcggctgcgcggcaccgacggcaaggcgccaggcatgatcgggtggtggccagccaaggcggtgaccttcgtggacaaccatgacaccggctccacgcagcacatgtggcccttcccttccgacaaggtcatgcagggatacgcctacatcctcacgcaccctgggaccccatgcatcttctacgatcatttcttcgactgggggctgaaggaggagatcgatcgcctggtgtcaatcaggacccggcaggggatacacagtgagagcaagctgcaaatcatagaggccgacgccgacctttacctggccgagatcgatggcaaggtcatcgtcaagctcgggccaagatacgatgtcgggcacctcattcctcaaggcttcaaggtggtcgcgcacggcaatgactatgccgtatgggagaaaatataa

SEQ ID NO: 12 - protein, 427 aa, unidentified
  source 1..427: note = Triticum aestivum L.
MANKHSSLSLFLVLLGLSASLASGQVLFQGFNWESWKHNGGWYNFLMGKVDDIAAAGVTHVWLPPASQSVAEQGYMPGRLYDLDASKYGNKAQLKSLIGALHGKGVKAIADIVINHRTAERKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEPSFAVAEIWTSLAYGGDGKPNLNQDQHRQELVNWVNKVGGSGPGTTFDFTTKGILNVAVEGELWRLRGTDGKAPGMIGWWPAKAVTFVDNHDTGSTQHMWPFPSDKVMQGYAYILTHPGTPCIFYDHFFDWGLKEEIDRLVSIRTRQGIHSESKLQIIEADADLYLAEIDGKVIVKLGPRYDVGHLIPQGFKVVAHGNDYAVWEKI

SEQ ID NO: 13 - DNA, 1650 bp, unidentified
  source 1..1650: mol_type = unassigned DNA; note = Triticum aestivum L.
atggcgaacaaacacttgtccctcttcctcgtcctccttggcctgtcggccagcttggcatccgggcaagtcctctttcaggtaagatcgtgtctgttgtctgtaactgtaagcattatcttcagatcgtaggtgccgcgatcatgtatgttttggaccgagttttgatccgagttctgtggtagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacggcggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcacccaggcaccccatgcatcgtgagtcatcctacaaatactacatcatatctaaattttcttttcttctttcgttcataaaaatataaaaccatgaccgaactgatgaaaaataattgtgattctttagttctacgatcatttcttcgactggggtctgaaggacgagatcgatcgcctggtgtcaatcaggacccggcatgggatacacagtgaaagcaagctgcaaatcatagaggccgacgccgacctttatctcgccgagatcgacggcaaggtcatcgtcaaggtcgggccaagatatgatgtgggacacctcattccgggaggcttcaaggtggccgcgcatggcaatgactatgcggtatgggagaaaatatgagcaaaattgccgaaggagctctacaattttttccctatgtgtacgtgatacatattggtccgagctcacgttgtccacatagtacgatttagtacttcctccatgtaaaagtgaggacgaggggcatccattgtattctcaataaataataatcatttc

SEQ ID NO: 14 - DNA, 1437 bp, unidentified
  source 1..1437: mol_type = unassigned DNA; note = Triticum aestivum L.
atggcgaacaaacacttgtccctcttcctcgtcctccttggcctgtcggccagcttggcatccgggcaagtcctctttcagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacggcggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcacccaggcaccccatgcatcttctacgatcatttcttcgactggggtctgaaggacgagatcgatcgcctggtgtcaatcaggacccggcatgggatacacagtgaaagcaagctgcaaatcatagaggccgacgccgacctttatctcgccgagatcgacggcaaggtcatcgtcaaggtcgggccaagatatgatgtgggacacctcattccgggaggcttcaaggtggccgcgcatggcaatgactatgcggtatgggagaaaatatgagcaaaattgccgaaggagctctacaattttttccctatgtgtacgtgatacatattggtccgagctcacgttgtccacatagtacgatttagtacttcctccatgtaaaagtgaggacgaggggcatccattgtattctcaataaataataatcatttc

SEQ ID NO: 15 - DNA, 1278 bp, unidentified
  source 1..1278: mol_type = unassigned DNA; note = Triticum aestivum L.
atggcgaacaaacacttgtccctcttcctcgtcctccttggcctgtcggccagcttggcatccgggcaagtcctctttcagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacggcggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcacccaggcaccccatgcatcttctacgatcatttcttcgactggggtctgaaggacgagatcgatcgcctggtgtcaatcaggacccggcatgggatacacagtgaaagcaagctgcaaatcatagaggccgacgccgacctttatctcgccgagatcgacggcaaggtcatcgtcaaggtcgggccaagatatgatgtgggacacctcattccgggaggcttcaaggtggccgcgcatggcaatgactatgcggtatgggagaaaatatga

SEQ ID NO: 16 - protein, 425 aa, unidentified
  source 1..425: note = Triticum aestivum L.
MANKHLSLFLVLLGLSASLASGQVLFQGFNWESWKHNGGWYNFLMGKVDDIAAAGITHVWLPPASQSVAEQGYMPGRLYDLDASKYGNKAQLKSLIGALHGKGVKAIADIVINHRTAEHKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEPSFAVAEIWTSLAYGGDGKPNLNQDQHRQELVNWVDKVGGKGPATTFDFTTKGILNVAVEGELWRLRGTDGKAPGMIGWWPAKAVTFVDNHDTGSTQHMWPFPSDRVMQGYAYILTHPGTPCIFYDHFFDWGLKDEIDRLVSIRTRHGIHSESKLQIIEADADLYLAEIDGKVIVKVGPRYDVGHLIPGGFKVAAHGNDYAVWEKI

SEQ ID NO: 17 - DNA, 1724 bp, unidentified
  source 1..1724: mol_type = unassigned DNA; note = Triticum aestivum L.
tgaatgcatcagttctccatcttcctcttccaaagctcactagagagagctgaagaagatggcgaacaaacacttgtccctcttcctcgtcctccttggcctgtcggccagcttggcatccgggcaagtcctctttcaggtaagatcgtgtctgttgtctgtaactgtaagcattatcttcagatcgcaggtgccgcgatcatgtatgttttggaccgagttttgatccgagttctgtggtagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgaaggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacgggggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcacccaggcaccccatgcatcgtgagtcatcctaccaatactacatcatatctaaattttcttttcttctttcgttcataaaaatataagaaaaccatgacccaactgatgaaaattaattgtgactcttcagttctacgatcatttcttcgactggggcctgaaggacgagatcgatcgcctggtgtcaatcaggacccgacatgggatacacagtgaaagcaagttgcaaatcatagaggccgacgccgatctttatctcgccgagatcgacggcaaggtcatcgtcaagctcgggccaagatacgatgtgggacacctcattccgggaggcttcaaggtgaccgcacacggcaatgactatgccgtatgggagaaaatatgagcaaaattgccgaaggagctctacaattttttccctatgtgtacgtgatacatattggtccgagctcatgttgtccacatagtatttatttagtacttcatccatgtaaaagtgaggacgaggggcatccattgtattctcaataaataataatcatttcatattttcagta

SEQ ID NO: 18 - DNA, 1508 bp, unidentified
  source 1..1508: mol_type = unassigned DNA; note = Triticum aestivum L.
tgaatgcatcagttctccatcttcctcttccaaagctcactagagagagctgaagaagatggcgaacaaacacttgtccctcttcctcgtcctccttggcctgtcggccagcttggcatccgggcaagtcctctttcagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgaaggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacgggggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcacccaggcaccccatgcatcttctacgatcatttcttcgactggggcctgaaggacgagatcgatcgcctggtgtcaatcaggacccgacatgggatacacagtgaaagcaagttgcaaatcatagaggccgacgccgatctttatctcgccgagatcgacggcaaggtcatcgtcaagctcgggccaagatacgatgtgggacacctcattccgggaggcttcaaggtgaccgcacacggcaatgactatgccgtatgggagaaaatatgagcaaaattgccgaaggagctctacaattttttccctatgtgtacgtgatacatattggtccgagctcatgttgtccacatagtatttatttagtacttcatccatgtaaaagtgaggacgaggggcatccattgtattctcaataaataataatcatttcatattttcagta

SEQ ID NO: 19 - DNA, 1278 bp, unidentified
  source 1..1278: mol_type = unassigned DNA; note = Triticum aestivum L.
atggcgaacaaacacttgtccctcttcctcgtcctccttggcctgtcggccagcttggcatccgggcaagtcctctttcagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgaaggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacgggggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcacccaggcaccccatgcatcttctacgatcatttcttcgactggggcctgaaggacgagatcgatcgcctggtgtcaatcaggacccgacatgggatacacagtgaaagcaagttgcaaatcatagaggccgacgccgatctttatctcgccgagatcgacggcaaggtcatcgtcaagctcgggccaagatacgatgtgggacacctcattccgggaggcttcaaggtgaccgcacacggcaatgactatgccgtatgggagaaaatatga

SEQ ID NO: 20 - protein, 425 aa, unidentified
  source 1..425: note = Triticum aestivum L.
MANKHLSLFLVLLGLSASLASGQVLFQGFNWESWKHNGGWYNFLMGKVDDIAAAGITHVWLPPASQSVAEQGYMPGRLYDLDASKYGNKAQLKSLIGALHGKGVKAIADIVINHRTAEHKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEPSFAVAEIWTSLAYGGDGKPNLNQDQHRQELVNWVDKVGGKGPATTFDFTTKGILNVAVEGELWRLRGTDGKAPGMIGWWPAKAVTFVDNHDTGSTQHMWPFPSDRVMQGYAYILTHPGTPCIFYDHFFDWGLKDEIDRLVSIRTRHGIHSESKLQIIEADADLYLAEIDGKVIVKLGPRYDVGHLIPGGFKVTAHGNDYAVWEKI

SEQ ID NO: 21 - DNA, 2906 bp, unidentified
  source 1..2906: mol_type = unassigned DNA; note = Triticum aestivum L.
agtctggggtgaacaataaaatcgcaaaacaatagcaaaaagaatcaaaaaaatctgaaattttagagcatcgaagatactttggtgcgcaagacacgttcactgatttactgttcatcccggactaaaaccgcccaaagcggcacgagggacgaaaactatccggttttgatagctgagggacgaaaactctccggtttagagttgagggacgatttctaaactctgggaagagtttgaggacgaaaaatatacttatccctttagtagaggatgatttgttctaaaaacaagtagatgatgatttgtaagaaagcacactgttccatgaaaaatgctcaaatgttctgtcctcagaaaaaacagaggtacagagtaactgacagtcgttttggccggtgccttcttatggaaggcgaaggcctgctccatcatttggccattaaatcgccttttgaccgcaccggccgataacaaactccggccgacatatccactggcccaaaggagcattcaagccgagcacgccgccttccggcatgctgcagcacattataaatacctggccagacacaccagctgaatgcatcagttctccatcttcctcttccaaagctcagctagagagagctgaagaagatggcgaacaaacacttgtccctcttcctcgtcctccttggcctgtcggccagcttggcatccgggcaagtcctctttcaggtaagatcgtgtctgttgtctgtaactgtaagcattatcttcagatcgtaggtgccgcgatcatgtatgttttggaccgagttttgatccgagttctgtggtagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacagcggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcatccaggcaccccatgcatcgtgagtcatcctaccaatactacatcatatctaaattttcttttcttctttcgttcataaaaatataagaaaaccatgacccaactgatgaaaattaattgtgactcttcagttctacgatcatttcttcgactggggcctgaaggacgagatcgatcgcctggtgtcaatcaggacccgacatgggatacacagtgaaagcaagctgcaaatcatagaggccgacgccgatctttatctcgccgagatcgatggcaaggtcatcgtcaagctcgggccaagatacgatgtgggacacctcattccgggaggcttcaaggtggccgcacacggcaatgactatgccgtatgggagaaaatatgagaaaaattgccgaaggagctctacaattttttctctatgtgtacgtgatacatattggtccgagctcacgttgtccacatagtacgattagtacttcctccatgtaaaagtgaggatgagggacatccattgtatttttgctaaaataataatcaataaggattttcgcactcttggcttacttagtgttggttgaatgaaaactgagcagccacgaccagtagctcgtacggcagcatactggagtagtggtagagcgttttgttacggaattgtgttacgggggctgggtttaaaggacacatactaatagtctggtgactgaaaatttatttttggccagtcacgttaaattcagattaaacaaacacaaagcaatcatatacaacaaaaagttgactggccaaaattttggttcagtcgaattcctcacacagcctaactcatgcaattttttttatttgcacacaaaaacgatgtgaggcccaaaaacaccagttgggctgatgcagcctacaagaacgctaagagcatcttcagccgttggccccccaggaggcataaaaatcgccgcttgggggtgagccggcggcaaaaccggctcttggggcggttgggcacccagccgtcgcccccaagcgccgatttcggcccattattcggcccattttcatcgaagaattgcttgatattggcgcgaatcggcccatattcgacgcggttcggtgtgttccagagggaattctccataaatattttttttgccttcatag

SEQ ID NO: 22 - DNA, 1490 bp, unidentified
  source 1..1490: mol_type = unassigned DNA; note = Triticum aestivum L.
cttcctcttccaaagctcagctagagagagctgaagaagatggcgaacaaacacttgtccctcttcctcgtcctccttggcctgtcggccagcttggcatccgggcaagtcctctttcagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacagcggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcatccaggcaccccatgcatcttctacgatcatttcttcgactggggcctgaaggacgagatcgatcgcctggtgtcaatcaggacccgacatgggatacacagtgaaagcaagctgcaaatcatagaggccgacgccgatctttatctcgccgagatcgatggcaaggtcatcgtcaagctcgggccaagatacgatgtgggacacctcattccgggaggcttcaaggtggccgcacacggcaatgactatgccgtatgggagaaaatatgagaaaaattgccgaaggagctctacaattttttctctatgtgtacgtgatacatattggtccgagctcacgttgtccacatagtacgattagtacttcctccatgtaaaagtgaggatgagggacatccattgtatttttgctaaaataataatcaataaggattttcgcactc

SEQ ID NO: 23 - DNA, 1278 bp, unidentified
  source 1..1278: mol_type = unassigned DNA; note = Triticum aestivum L.
atggcgaacaaacacttgtccctcttcctcgtcctccttggcctgtcggccagcttggcatccgggcaagtcctctttcagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacagcggtgacggcaagcccaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacagggtcatgcagggatatgcctacatcctcacgcatccaggcaccccatgcatcttctacgatcatttcttcgactggggcctgaaggacgagatcgatcgcctggtgtcaatcaggacccgacatgggatacacagtgaaagcaagctgcaaatcatagaggccgacgccgatctttatctcgccgagatcgatggcaaggtcatcgtcaagctcgggccaagatacgatgtgggacacctcattccgggaggcttcaaggtggccgcacacggcaatgactatgccgtatgggagaaaatatga

SEQ ID NO: 24 - protein, 425 aa, unidentified
  source 1..425: note = Triticum aestivum L.
MANKHLSLFLVLLGLSASLASGQVLFQGFNWESWKHNGGWYNFLMGKVDDIAAAGITHVWLPPASQSVAEQGYMPGRLYDLDASKYGNKAQLKSLIGALHGKGVKAIADIVINHRTAEHKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEPSFAVAEIWTSLAYSGDGKPNLNQDQHRQELVNWVDKVGGKGPATTFDFTTKGILNVAVEGELWRLRGTDGKAPGMIGWWPAKAVTFVDNHDTGSTQHMWPFPSDRVMQGYAYILTHPGTPCIFYDHFFDWGLKDEIDRLVSIRTRHGIHSESKLQIIEADADLYLAEIDGKVIVKLGPRYDVGHLIPGGFKVAAHGNDYAVWEKI

SEQ ID NO: 25 - DNA, 2859 bp, unidentified
  source 1..2859: mol_type = unassigned DNA; note = Triticum aestivum L.
agatttatgtcagttttatccgcctcgaggaacgcacctcatccaccttatccaggaaattttgccaggaattttctgacccggatttctgtttgttaacctgaactgcgcaagtactcgtcagttgcccgcagatctcacgaactgctacggcacttgtcaactgaccctcatttcagtgcgccagagctgaaaccctttcggcagcaaaaccttcccaattttcttggtttgatctacactgttttagtagatgatggttggtaaccacacacacacacacacacgattccatttccactgctagaaaggacggtatttatattcccctagtttggcgtgtgagttctgaggaattctgaagctcagcaggataactgacagccgtattggccggtgccttctcatacaaggctggctccatcactcgccttttgagctcaccgcaccggccgataacaaactccggccgacatatccatcgatccaacggagcattgaagtcgatcgcaccggaacatgtcttgcaagttgcccaccggcatgctgcagcacagtataaatacctggccagacacaccagctgaatccaccagttctccatcctcctctacgagagcacagctagagagagctgaagaacatggcgagcaaacacttatccctcttcctcgtcctccttggcctgtcggccagtttggcctctggccaagtcctgtttcaggtaagatcgtgtcctgtcttcagctcctatacgtaccgtgatcatgtttcggggactgagttttgagttgtgacttgtgcgtgcggcagggtttcaactgggagtcgtggaagcacaatggcggatggtataacttcctgatgggcaaggtggacgacatcgccgctgccggcgtcacgcacgtctggctccccccggcgtcgcagtccgtctccgagcaagggtacatgccgggccggctgtacgacctggacgcctccaagtacggcaacaaggcgcagctcaagtcactgatcggggcgctccatggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacatcggcttcgacggctggcgcttcgatttcgccaagggctactccgcggacgtggccaagatctacgtcgaccgctccgaggccagcttcgccgtggccgagatatggacgtcgctggcgtacggcggggacggcaagccgaacctcaaccaggacccgcaccggcaggagctggtgaactgggtgaacaaggtgggcggctccggcccgggcaccacgttcgacttcaccaccaagggcatcctcaacgtcgccgtggagggcgagctgtggcggctgcgcggcaccgacggcaaggcgccgggcatgatcgggtggtggccagccaaggcggtgaccttcgtcgacaaccacgacaccggctccacgcagcacatgtggcccttcccttctgacagggtcatgcagggatacgcctacatcctcacgcacccagggaccccatgcatcgtgagtcatcctacctgttcatcgtatctaaattggtcttcttcttttttcgttcatataagaaaccatgcatgaccgaactgatgaaaaataattgtgattcttcagttctacgatcatttcttcgactggggcctgaaggaggagatagatcgcctagtgtcaatcaggacccggcaaggcatacacagtgagagcaagctgcaaatcatagaggctgacgccgacctttaccttgccgagatcgatggcaaggtcatcgtcaagctcgggccaagatatgatgtggggcacctcattcccggaggcttcaaggtggccgcacacggcaaagactatgccatatgggagaaaatataagaaaattacgggagcagctctgcaaattagtcaagttgtccacatagtacgattttagtacttcctccatgtaaaagtgaggatgagggacatccattgtatttttcataaataataatcaataagtgttttcgatactcatttttcgttgaacaaaaaatgtgtagagcacggccgtgcgggagcgtgcaacctaaagcttgaggctttgatggtaggagctccgcctttgcattaaggattagggctcttctaggatcagatcccgaccagaaggggcagggggtgaatgggagttttgaccatttcttcaaaaaagcaagttttctagcaaaaacagggtacgtaaaacaatagcaaaacagaatagagaataaactaaacagaacagagaaataacttagagaaattatacatgaacagaggtaaatctaagtaggaatgcacacaaaactaaacatggcataggcaacggaataacaaagtggtcaaaatcatcttcacggcaatcacagagaaaaaagacagggagaagttcttcgaacacatgaagttacataagtaaatgagtttgataaaagtacaaaaatacatgtaagcttgatgaagacatatttgttcgaccaatctgagttgttggcacaactctacgatctgtttggaggggccgagataaaactcaaaagacaatgtcttcatcctatgctccttgagataaagtcacttagactccactcaatc

SEQ ID NO: 26 - DNA, 1462 bp, unidentified
  source 1..1462: mol_type = unassigned DNA; note = Triticum aestivum L.
ccatcctcctctacgagagcacagctagagagagctgaagaacatggcgagcaaacacttatccctcttcctcgtcctccttggcctgtcggccagtttggcctctggccaagtcctgtttcagggtttcaactgggagtcgtggaagcacaatggcggatggtataacttcctgatgggcaaggtggacgacatcgccgctgccggcgtcacgcacgtctggctccccccggcgtcgcagtccgtctccgagcaagggtacatgccgggccggctgtacgacctggacgcctccaagtacggcaacaaggcgcagctcaagtcactgatcggggcgctccatggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacatcggcttcgacggctggcgcttcgatttcgccaagggctactccgcggacgtggccaagatctacgtcgaccgctccgaggccagcttcgccgtggccgagatatggacgtcgctggcgtacggcggggacggcaagccgaacctcaaccaggacccgcaccggcaggagctggtgaactgggtgaacaaggtgggcggctccggcccgggcaccacgttcgacttcaccaccaagggcatcctcaacgtcgccgtggagggcgagctgtggcggctgcgcggcaccgacggcaaggcgccgggcatgatcgggtggtggccagccaaggcggtgaccttcgtcgacaaccacgacaccggctccacgcagcacatgtggcccttcccttctgacagggtcatgcagggatacgcctacatcctcacgcacccagggaccccatgcatcttctacgatcatttcttcgactggggcctgaaggaggagatagatcgcctagtgtcaatcaggacccggcaaggcatacacagtgagagcaagctgcaaatcatagaggctgacgccgacctttaccttgccgagatcgatggcaaggtcatcgtcaagctcgggccaagatatgatgtggggcacctcattcccggaggcttcaaggtggccgcacacggcaaagactatgccatatgggagaaaatataagaaaattacgggagcagctctgcaaattagtcaagttgtccacatagtacgattttagtacttcctccatgtaaaagtgaggatgagggacatccattgtatttttcataaataataatcaataagtgttttcgatactca

SEQ ID NO: 27 - DNA, 1278 bp, unidentified
  source 1..1278: mol_type = unassigned DNA; note = Triticum aestivum L.
atggcgagcaaacacttatccctcttcctcgtcctccttggcctgtcggccagtttggcctctggccaagtcctgtttcagggtttcaactgggagtcgtggaagcacaatggcggatggtataacttcctgatgggcaaggtggacgacatcgccgctgccggcgtcacgcacgtctggctccccccggcgtcgcagtccgtctccgagcaagggtacatgccgggccggctgtacgacctggacgcctccaagtacggcaacaaggcgcagctcaagtcactgatcggggcgctccatggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacatcggcttcgacggctggcgcttcgatttcgccaagggctactccgcggacgtggccaagatctacgtcgaccgctccgaggccagcttcgccgtggccgagatatggacgtcgctggcgtacggcggggacggcaagccgaacctcaaccaggacccgcaccggcaggagctggtgaactgggtgaacaaggtgggcggctccggcccgggcaccacgttcgacttcaccaccaagggcatcctcaacgtcgccgtggagggcgagctgtggcggctgcgcggcaccgacggcaaggcgccgggcatgatcgggtggtggccagccaaggcggtgaccttcgtcgacaaccacgacaccggctccacgcagcacatgtggcccttcccttctgacagggtcatgcagggatacgcctacatcctcacgcacccagggaccccatgcatcttctacgatcatttcttcgactggggcctgaaggaggagatagatcgcctagtgtcaatcaggacccggcaaggcatacacagtgagagcaagctgcaaatcatagaggctgacgccgacctttaccttgccgagatcgatggcaaggtcatcgtcaagctcgggccaagatatgatgtggggcacctcattcccggaggcttcaaggtggccgcacacggcaaagactatgccatatgggagaaaatataa

SEQ ID NO: 28 - protein, 425 aa, unidentified
  source 1..425: note = Triticum aestivum L.
MASKHLSLFLVLLGLSASLASGQVLFQGFNWESWKHNGGWYNFLMGKVDDIAAAGVTHVWLPPASQSVSEQGYMPGRLYDLDASKYGNKAQLKSLIGALHGKGVKAIADIVINHRTAERKDGRGIYCIFEGGTPDARLDWGPHMICRDDRPYADGTGNPDTGADFGAAPDIDHLNPRVQKELVEWLNWLRTDIGFDGWRFDFAKGYSADVAKIYVDRSEASFAVAEIWTSLAYGGDGKPNLNQDPHRQELVNWVNKVGGSGPGTTFDFTTKGILNVAVEGELWRLRGTDGKAPGMIGWWPAKAVTFVDNHDTGSTQHMWPFPSDRVMQGYAYILTHPGTPCIFYDHFFDWGLKEEIDRLVSIRTRQGIHSESKLQIIEADADLYLAEIDGKVIVKLGPRYDVGHLIPGGFKVAAHGKDYAIWEKI

SEQ ID NO: 29 - DNA, 2842 bp, unidentified
  source 1..2842: mol_type = unassigned DNA; note = Triticum aestivum L.
gtactactcaccagaaatttatgtccagttttatccgcctcgagggacgcacctcatccaggttatccaggaatttgctcacccggattttagctgcctgaaatgcgcaagtaaccgcggagccatctcgccaaagttaccgtcagttgcccctgatcttacgaactgttgttacagcacaactgaccctcattgcagtgcgagaaagccgaaaccttttcgattgatatcacgcccttttagtagacgatggctcgtaaccacacacacactattccatttttccactgctagaaaggacggtgtttattttcccctagctagtttggcgtgtgagtgagttctgaggaattctcaagttcagcaggataactgactgtcgtattggccggtgcctttctcatcgaaggctggctccatcactcgccttttgagctcaccgcaccggccgataacaaactccggccgacatatccatcgatccaacggagcattgaagtcgatcgcaccggaacatgtcttgcaagttgcccaccggcatgctgcagcacagtataaatacctggccagacacacaagctgaatccatcagttctccatcgccttcttccaaagcacagctcgctagagctcaagatcatggcgaacaaacactcgtccctccccctcttcctcgtcctccttggcctgtcggccagcttggcctccgggcaactcctgtttcaggtaagaataagatcttgtacccgtcggcaagctcctgggttttggaccgagttttgacttctgctgagtgcggcagggtttcaactgggagtcgtggaagcacaacggcgggtggtacaacttcctgatgggcaaggtggacgacatcgccgcggccgggatcacgcacgtctggctccctccggcgtcgcagtccgtcgccgagcaagggtacatgccgggccggctctacgacctggacgcgtccaagtacggcaacaaagcgcagctcaagtccctcatcggggcgctccacggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacgtcggcttcgacggctggcgcttcgacttcgccaagggctactcggcggacgtcgccaagatctacatcgaccgctccgaggccagcttcgccgtggccgagatatggacgtcgctggcgtacggcggggacggcaagccgaacctcaaccaggaccagcaccggcaggagctggtgaactgggtgaacaaggtgggcggctccggcccgggcaccacgttcgacttcaccaccaagggcatcctcaacgtcgccgtggagggcgagctgtggcggctgcgcggcacagacggcaaggcgccaggcatgatcgggtggtggccagccaaggcggtgaccttcgtcgacaaccatgacaccggctccacgcagcacatgtggcccttcccttccgacagggtcatgcagggatacgcctacatcctcacgcacccagggaccccatgcatcgtgagtcatcctaccagttcatcgtatctaaattgctcttcttcctttttcgttcatataagaaaccatggatgaccgaactgatgaaaaataattgtgattcttcagttctacgatcatttcttcgactggggcctaaaggaggagatcgatcgcctagtgtcaatcaggaccaggcagggcatacacagtgagagcaagctgcaaatcatagaggctgacgccaatctttaccttgccgagatcgacggcaaggtcatcgtcaagctcgggccaagatacgatgtggggcacctcattcccggaggctttaaggtggccgcacacggcaaagactatgctgtatgggagaaaatataagaaaattaccggagcagcttgcaaattagtcatgttgtccacatagtacgattttagtacttcctccatgtaaaagtgaggatgagggacatccattgtatttttcataaataataataaatagtgttttcgctactcatggtttattcattgttcgttgaccaaaaattgcgtacagcacgaccgtgcgggagcgtgcaacctaaagcttgaggctttgatggtaggagctctgcctttgcatcgaatatcgaccaaaggggggggggggtgaatgggaacttcgacaatttctttagaaaaacgagttttttgagcaaaaacagagtacatagaagaactatagatcagaataaagaataaactaaactgaaaagagaattaactgaggttaatctaagtagcaatgcacacaaaactaaacatgtcataggcagcagaataacaaagtgctcaaaatcatcttcgcagaaatcacaacagtcacagagaaataagacggggagaagttcttcgaatgcatgcacgttacataggtaaatgagttcgataaagtaaaaaacctgtaagcttgatgaagacatagatttgttcgaccagtccaagttgctagcacaactctatgatctgtttggaggggccgagattaaactcagaagacaatgtcttcatcctatattccttgagataaaacacttagacttcgctcaatcactcgtggtagatctccgaaac

SEQ ID NO: 30 - DNA, 1458 bp, unidentified
  source 1..1458: mol_type = unassigned DNA; note = Triticum aestivum L.
cgccttcttccaaagcacagctcgctagagctcaagatcatggcgaacaaacactcgtccctccccctcttcctcgtcctccttggcctgtcggccagcttggcctccgggcaactcctgtttcagggtttcaactgggagtcgtggaagcacaacggcgggtggtacaacttcctgatgggcaaggtggacgacatcgccgcggccgggatcacgcacgtctggctccctccggcgtcgcagtccgtcgccgagcaagggtacatgccgggccggctctacgacctggacgcgtccaagtacggcaacaaagcgcagctcaagtccctcatcggggcgctccacggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacgtcggcttcgacggctggcgcttcgacttcgccaagggctactcggcggacgtcgccaagatctacatcgaccgctccgaggccagcttcgccgtggccgagatatggacgtcgctggcgtacggcggggacggcaagccgaacctcaaccaggaccagcaccggcaggagctggtgaactgggtgaacaaggtgggcggctccggcccgggcaccacgttcgacttcaccaccaagggcatcctcaacgtcgccgtggagggcgagctgtggcggctgcgcggcacagacggcaaggcgccaggcatgatcgggtggtggccagccaaggcggtgaccttcgtcgacaaccatgacaccggctccacgcagcacatgtggcccttcccttccgacagggtcatgcagggatacgcctacatcctcacgcacccagggaccccatgcatcttctacgatcatttcttcgactggggcctaaaggaggagatcgatcgcctagtgtcaatcaggaccaggcagggcatacacagtgagagcaagctgcaaatcatagaggctgacgccaatctttaccttgccgagatcgacggcaaggtcatcgtcaagctcgggccaagatacgatgtggggcacctcattcccggaggctttaaggtggccgcacacggcaaagactatgctgtatgggagaaaatataagaaaattaccggagcagcttgcaaattagtcatgttgtccacatagtacgattttagtacttcctccatgtaaaagtgaggatgagggacatccattgtatttttcataaataataataaatagtgttttcgcta

SEQ ID NO: 31 - DNA, 1284 bp, unidentified
  source 1..1284: mol_type = unassigned DNA; note = Triticum aestivum L.
atggcgaacaaacactcgtccctccccctcttcctcgtcctccttggcctgtcggccagcttggcctccgggcaactcctgtttcagggtttcaactgggagtcgtggaagcacaacggcgggtggtacaacttcctgatgggcaaggtggacgacatcgccgcggccgggatcacgcacgtctggctccctccggcgtcgcagtccgtcgccgagcaagggtacatgccgggccggctctacgacctggacgcgtccaagtacggcaacaaagcgcagctcaagtccctcatcggggcgctccacggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacgtcggcttcgacggctggcgcttcgacttcgccaagggctactcggcggacgtcgccaagatctacatcgaccgctccgaggccagcttcgccgtggccgagatatggacgtcgctggcgtacggcggggacggcaagccgaacctcaaccaggaccagcaccggcaggagctggtgaactgggtgaacaaggtgggcggctccggcccgggcaccacgttcgacttcaccaccaagggcatcctcaacgtcgccgtggagggcgagctgtggcggctgcgcggcacagacggcaaggcgccaggcatgatcgggtggtggccagccaaggcggtgaccttcgtcgacaaccatgacaccggctccacgcagcacatgtggcccttcccttccgacagggtcatgcagggatacgcctacatcctcacgcacccagggaccccatgcatcttctacgatcatttcttcgactggggcctaaaggaggagatcgatcgcctagtgtcaatcaggaccaggcagggcatacacagtgagagcaagctgcaaatcatagaggctgacgccaatctttaccttgccgagatcgacggcaaggtcatcgtcaagctcgggccaagatacgatgtggggcacctcattcccggaggctttaaggtggccgcacacggcaaagactatgctgtatgggagaaaatataa

SEQ ID NO: 32 - protein, 427 aa, unidentified
  source 1..427: note = Triticum aestivum L.
MANKHSSLPLFLVLLGLSASLASGQLLFQGFNWESWKHNGGWYNFLMGKVDDIAAAGITHVWLPPASQSVAEQGYMPGRLYDLDASKYGNKAQLKSLIGALHGKGVKAIADIVINHRTAERKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEASFAVAEIWTSLAYGGDGKPNLNQDQHRQELVNWVNKVGGSGPGTTFDFTTKGILNVAVEGELWRLRGTDGKAPGMIGWWPAKAVTFVDNHDTGSTQHMWPFPSDRVMQGYAYILTHPGTPCIFYDHFFDWGLKEEIDRLVSIRTRQGIHSESKLQIIEADANLYLAEIDGKVIVKLGPRYDVGHLIPGGFKVAAHGKDYAVWEKI

SEQ ID NO: 33 - DNA, 2839 bp, unidentified
  source 1..2839: mol_type = unassigned DNA; note = Triticum aestivum L.
tttcccagccacatactcgtaccctcacgaattcaatgctcgtcgaacgaatgaatgctcggatatatgttgatctctccggactgaaaaaaagttcgaaccgggtactcaccagaaagtttttgaacaatgctggcagatttatgtcagttttatccgcctcgaggagcgcacctcatccacgttatccaggaaattttggcaggaattttctgacccggatttctgtttgttaacctgaaaaatgcgaaagtaaccgtcagttgcccgcagatcttacgaaccgctacagcacttggatgtttatttacccccattttggcgtgtgagttctgaggaattctcacgttcagcaggataactggcagccgtattggccggtgccttctgatacaaggctagctccgtcactcgccttttgagctcaccgttccggccgataacaaactccggccgacatatccatcgatccaacggagcattgaagtcgatcgcaccggaacatgtcttgcaagttgctcgatcaccggcatgatgcatcctgcagcacactataaatacctggccagacacaccagctgaatccatcagttctccatcctcctcttccagagcacagtagagagggctgaagaacatggcgaacaaacactcgtccctccccctcttcctcgtcctccttggcctgtcggccagcttggcctccggtcaaattctctttcaggtattatcttcttcgtcggcaagaactgtcttcaggctgcgatcatgttttgttttggactgagttttgagttctgctgcgtgcggcagggtttcaactgggagtcgtggaagcacaatggcgggtggtacaacttcctgatgggcaaggtggacgacatcgccgccggcggcatcacgcacgtgtggctccccccggcgtcgcagtccgtggccgagcaaggctacatgccgggccggctgtacgacctggacgcctccaagtacggcaacaaggcgcagctcaagtccctgatcggggcgctccacggcaagggcgtcaaggccatcgccgacattgtcatcaaccaccgcacggcggagcgcaaggacggccggggcatctactgcatcttcgagggcggcacccccgacgcgcgccttgactggggcccccacatgatctgccgcgacgaccggccctacgccgacggcacgggcaacccggacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctccgaggccagcttcgccgtggccgagatatggacgtcgctggcgtacggcggggacggcaagcccaacctcaaccaggacccgcaccggcaggagctggtgaactgggtgaacaaggtgggcggctccggccccggcaccacgttcgacttcaccaccaagggcatcctcaatgtggccgtggagggcgagctgtggcggctgcgcggcactgacggcaaggcgccgggcatgatcgggtggtggccagccaaggcagtgaccttcgtggacaaccacgacaccggctccacgcaacacatgtggcccttcccttctgacagggtcatgcagggatatgcctacatcctcacgcacccagggaccccatgcatcgtgagtcatcgtgctattgccaactcatcatatctaaattgtccttttttctccgttcatatgaaaccatgaccaaactgatgaaaactaaatgtgattcttcagttctacgatcatttcttcgactggggcctgaaggaggagatcgatcgcctggtgtcaatcaggacccgacaggggatacacagtgagagcaagctacaaatcatagaggctgacgccgacctttacctagccgagatcgacagcaaggtcatcgtcaaactcgggccaagatacgatgtcgggcacctcattcccggaggcttcaaggtggtcgcgcacggcaaagattatgccgtatgggagaaaatataagcaaaattaccggagctcatgatgtccacgtagtacgatttagtacttcctccgtgtaaaagtgaggatgagggacatccattgtattttttatataaataataatcaataagtatttccgctactcatggtttactcgatgtttgttgaccataaattatgcagcgcacggccagtgactcgtgcggaagcatgcaacctccaagcttgatgcttggggtggtaggagctctgcctttgcattaaggattagggctctacataagaaagcatgggaaaggagaagaagaaatcttcacaaggcgatgtctgtagcttagttatacaatgctccagctatcagttctgaatcaactttgatcagagctgctatgtgttggagaaaatctgattatttctttttttttttcaaaaaaaaaccccgacgctttctttcattcaaaatatgtgccgagcaaagtataatgccgcggttgcattcttcttcgggcgagagaagaggtggtaccaccattcttggatggaactgaagctgttcatctaagttgcaccaaagtgtggcatgttgcagctaagtttgaacgctgtggcgaacggatagcggtggattagtgagctgcaaattctagtagctggtcacaaagaaagcaaacatggttgtaatcgcagcctcctgcatgcggtctgtcagtggtgggaagccaattttgtactacactatttg

SEQ ID NO: 34 - DNA, 1445 bp, unidentified
  source 1..1445: mol_type = unassigned DNA; note = Triticum aestivum L.
ctcctcttccagagcacagtagagagggctgaagaacatggcgaacaaacactcgtccctccccctcttcctcgtcctccttggcctgtcggccagcttggcctccggtcaaattctctttcagggtttcaactgggagtcgtggaagcacaatggcgggtggtacaacttcctgatgggcaaggtggacgacatcgccgccggcggcatcacgcacgtgtggctccccccggcgtcgcagtccgtggccgagcaaggctacatgccgggccggctgtacgacctggacgcctccaagtacggcaacaaggcgcagctcaagtccctgatcggggcgctccacggcaagggcgtcaaggccatcgccgacattgtcatcaaccaccgcacggcggagcgcaaggacggccggggcatctactgcatcttcgagggcggcacccccgacgcgcgccttgactggggcccccacatgatctgccgcgacgaccggccctacgccgacggcacgggcaacccggacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctccgaggccagcttcgccgtggccgagatatggacgtcgctggcgtacggcggggacggcaagcccaacctcaaccaggacccgcaccggcaggagctggtgaactgggtgaacaaggtgggcggctccggccccggcaccacgttcgacttcaccaccaagggcatcctcaatgtggccgtggagggcgagctgtggcggctgcgcggcactgacggcaaggcgccgggcatgatcgggtggtggccagccaaggcagtgaccttcgtggacaaccacgacaccggctccacgcaacacatgtggcccttcccttctgacagggtcatgcagggatatgcctacatcctcacgcacccagggaccccatgcatcttctacgatcatttcttcgactggggcctgaaggaggagatcgatcgcctggtgtcaatcaggacccgacaggggatacacagtgagagcaagctacaaatcatagaggctgacgccgacctttacctagccgagatcgacagcaaggtcatcgtcaaactcgggccaagatacgatgtcgggcacctcattcccggaggcttcaaggtggtcgcgcacggcaaagattatgccgtatgggagaaaatataagcaaaattaccggagctcatgatgtccacgtagtacgatttagtacttcctccgtgtaaaagtgaggatgagggacatccattgtattttttatataaataataatcaataagtatttccgcta

SEQ ID NO: 35 - DNA, 1284 bp, unidentified
  source 1..1284: mol_type = unassigned DNA; note = Triticum aestivum L.
atggcgaacaaacactcgtccctccccctcttcctcgtcctccttggcctgtcggccagcttggcctccggtcaaattctctttcagggtttcaactgggagtcgtggaagcacaatggcgggtggtacaacttcctgatgggcaaggtggacgacatcgccgccggcggcatcacgcacgtgtggctccccccggcgtcgcagtccgtggccgagcaaggctacatgccgggccggctgtacgacctggacgcctccaagtacggcaacaaggcgcagctcaagtccctgatcggggcgctccacggcaagggcgtcaaggccatcgccgacattgtcatcaaccaccgcacggcggagcgcaaggacggccggggcatctactgcatcttcgagggcggcacccccgacgcgcgccttgactggggcccccacatgatctgccgcgacgaccggccctacgccgacggcacgggcaacccggacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctccgaggccagcttcgccgtggccgagatatggacgtcgctggcgtacggcggggacggcaagcccaacctcaaccaggacccgcaccggcaggagctggtgaactgggtgaacaaggtgggcggctccggccccggcaccacgttcgacttcaccaccaagggcatcctcaatgtggccgtggagggcgagctgtggcggctgcgcggcactgacggcaaggcgccgggcatgatcgggtggtggccagccaaggcagtgaccttcgtggacaaccacgacaccggctccacgcaacacatgtggcccttcccttctgacagggtcatgcagggatatgcctacatcctcacgcacccagggaccccatgcatcttctacgatcatttcttcgactggggcctgaaggaggagatcgatcgcctggtgtcaatcaggacccgacaggggatacacagtgagagcaagctacaaatcatagaggctgacgccgacctttacctagccgagatcgacagcaaggtcatcgtcaaactcgggccaagatacgatgtcgggcacctcattcccggaggcttcaaggtggtcgcgcacggcaaagattatgccgtatgggagaaaatataa

SEQ ID NO: 36 - protein, 427 aa, unidentified
  source 1..427: note = Triticum aestivum L.
MANKHSSLPLFLVLLGLSASLASGQILFQGFNWESWKHNGGWYNFLMGKVDDIAAGGITHVWLPPASQSVAEQGYMPGRLYDLDASKYGNKAQLKSLIGALHGKGVKAIADIVINHRTAERKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEASFAVAEIWTSLAYGGDGKPNLNQDPHRQELVNWVNKVGGSGPGTTFDFTTKGILNVAVEGELWRLRGTDGKAPGMIGWWPAKAVTFVDNHDTGSTQHMWPFPSDRVMQGYAYILTHPGTPCIFYDHFFDWGLKEEIDRLVSIRTRQGIHSESKLQIIEADADLYLAEIDSKVIVKLGPRYDVGHLIPGGFKVVAHGKDYAVWEKI

SEQ ID NO: 37 - DNA, 2875 bp, unidentified
  source 1..2875: mol_type = unassigned DNA; note = Triticum aestivum L.
  misc_difference 2364..2571: note = a, c, g, or t
gatgctggcagatttatgtccagttttacccgcctcgaggaacgcacctcatccaccttatccaggaaattttgccaggaattttctgacccggatttctgtttgttaacctgaactacgcaagtaagcatcagttgcccgcagatcttacgaactgctacagcacttctcaactgaccccggccccatttcactgcgcggaagctgaaatcttttcggttacaaaaccttctcaattttcttggcctgatttacactgttctcgtagatgatggttcgtgatcacacacactatctcatttccaccgctagaaactagaaaggatggtgtttattttcccctagctcacctagctagttttgcgtctgagttctgaggaattctgaagctcagcaggataactgacagccgtattggccggtgccttatcatacaaggctggctccatcactcgccttttgagctcaccgcaccggccgataacaaacggagcattgaagtcgagcgcaccggaacatgtcctgcaagttgctcaccggcatgcatgatgcatcctgcagcacactataaatacctggccagaggcaccagctgaatccatcagttctccatcctcctcttccaaagcacagctaactagagctcaagataatcatggcgaacaaacacccgtccctctccctcttcctcctcctcctcggcctctcgtccagcttggcctccggtcaagtcctgtttcaggtaagcataagatattgtccgccggcaagcgccgtctccggctcgtcggttttggactgagttttgagttgtgcgtgcggcagggtttcaactgggagtcgtggaagcacaatggcggatggtacaacttcctgatgggcaaggtggacgacatcgccgcggccggcgtcacgcacgtctggctgcccccggcgtcgcagtccgtctccaagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacggccggggcatctactgcatcttcgagggcggcacccccgacgcgcgcctcgactggggcccccacatgatctgccgcgacgaccggccctacgccgacggcaccggcaacccggacaccggcgccgacttcggggccgcgccggacatcgaccatctcaacccgcgcgtgcagaaggagctcgtcgagtggctcaactggctcaggaccgacgtcggcttcgacggctggcgcttcgacttcgccaagggctactccgccgacgtggccaagatctacatcgaccgctccgaggccagcttcgccgtggccgagatatggacgtcgctcgcgtacggcggggatgggaagcccaacctcaaccaggacccgcaccggcaggagctggtgaactgggtgaacaaggtgggcggctccggcacgggcaccacgttcgacttcaccaccaagggcatcctcaacgtcgccgtggagggcgagctgtggcggctgcgcggcaccgacggcaaggcgccgggcatgatcgggtggtggccagccaaggcggtgaccttcgtcgacaaccatgacaccggctccacgcagcatatgtggcccttcccttccgacagtgtcatgcagggatacgcgtacatcctcacgcacccagggaccccgtgcatcgtgagtcatcctaccagttcatcgtatctaaattggtcttcttcttttttcgttcataacaagaaaccatgcatgaccgaactgatgaaaaataattgtgactcttcagttctacgatcatttcttcgactggggcatgaaggacgagatctatcgcctagtgtcaatcaggacccggcaggggatacacagtgagagcaagctacaaatcatggaggctgacgccgacctttaccttgccgagatcgacggcaagatcatcgtcaagctcgggccacgattcgatgtggggcacctcattcccggaggcttcaaggtggccgcacacggcaaagactatgccgtatgggagaaaatataagaaaattaccagagcagctctgcaaattagtcatgttgtccacatagtacgattttagtacttcctccatgtaaaagtgaggatgagggacgtccattgcatttttcataaataataatcaataagcgttttgctattcatggtttactcaatgttcgttgaccaaaagttgcatagagcatgaccgtgtgggagcgtgcaacctaaagcttgaggttttgatggtacgagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacgagttttttcagcaataacagagtacatagaacaaatgcagatcagaatagagaataaactaaactgaaaagagaaataactgaggttaatataagtagcaatgcacacaaaactaaacatggcataggcagcggaatatcagagtgctcaaaatcgtcttcgtaacgatcacagcgaaataagacggggagaagttcttcgaacgcatgcatgttacataagtaaatgagttcgataaagtaaaaaaccctataagcttaacgaagacatagatttgttcgaccagtccgagttgctggc

SEQ ID NO: 38 - DNA, 1482 bp, unidentified
  source 1..1482: mol_type = unassigned DNA; note = Triticum aestivum L.
tcagttctccatcctcctcttccaaagcacagctaactagagctcaagataatcatggcgaacaaacacccgtccctctccctcttcctcctcctcctcggcctctcgtccagcttggcctccggtcaagtcctgtttcagggtttcaactgggagtcgtggaagcacaatggcggatggtacaacttcctgatgggcaaggtggacgacatcgccgcggccggcgtcacgcacgtctggctgcccccggcgtcgcagtccgtctccaagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacggccggggcatctactgcatcttcgagggcggcacccccgacgcgcgcctcgactggggcccccacatgatctgccgcgacgaccggccctacgccgacggcaccggcaacccggacaccggcgccgacttcggggccgcgccggacatcgaccatctcaacccgcgcgtgcagaaggagctcgtcgagtggctcaactggctcaggaccgacgtcggcttcgacggctggcgcttcgacttcgccaagggctactccgccgacgtggccaagatctacatcgaccgctccgaggccagcttcgccgtggccgagatatggacgtcgctcgcgtacggcggggatgggaagcccaacctcaaccaggacccgcaccggcaggagctggtgaactgggtgaacaaggtgggcggctccggcacgggcaccacgttcgacttcaccaccaagggcatcctcaacgtcgccgtggagggcgagctgtggcggctgcgcggcaccgacggcaaggcgccgggcatgatcgggtggtggccagccaaggcggtgaccttcgtcgacaaccatgacaccggctccacgcagcatatgtggcccttcccttccgacagtgtcatgcagggatacgcgtacatcctcacgcacccagggaccccgtgcatcttctacgatcatttcttcgactggggcatgaaggacgagatctatcgcctagtgtcaatcaggacccggcaggggatacacagtgagagcaagctacaaatcatggaggctgacgccgacctttaccttgccgagatcgacggcaagatcatcgtcaagctcgggccacgattcgatgtggggcacctcattcccggaggcttcaaggtggccgcacacggcaaagactatgccgtatgggagaaaatataagaaaattaccagagcagctctgcaaattagtcatgttgtccacatagtacgattttagtacttcctccatgtaaaagtgaggatgagggacgtccattgcatttttcataaataataatcaataagcgttttgctattcatggt

SEQ ID NO: 39 - DNA, 1284 bp, unidentified
  source 1..1284: mol_type = unassigned DNA; note = Triticum aestivum L.
atggcgaacaaacacccgtccctctccctcttcctcctcctcctcggcctctcgtccagcttggcctccggtcaagtcctgtttcagggtttcaactgggagtcgtggaagcacaatggcggatggtacaacttcctgatgggcaaggtggacgacatcgccgcggccggcgtcacgcacgtctggctgcccccggcgtcgcagtccgtctccaagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacggccggggcatctactgcatcttcgagggcggcacccccgacgcgcgcctcgactggggcccccacatgatctgccgcgacgaccggccctacgccgacggcaccggcaacccggacaccggcgccgacttcggggccgcgccggacatcgaccatctcaacccgcgcgtgcagaaggagctcgtcgagtggctcaactggctcaggaccgacgtcggcttcgacggctggcgcttcgacttcgccaagggctactccgccgacgtggccaagatctacatcgaccgctccgaggccagcttcgccgtggccgagatatggacgtcgctcgcgtacggcggggatgggaagcccaacctcaaccaggacccgcaccggcaggagctggtgaactgggtgaacaaggtgggcggctccggcacgggcaccacgttcgacttcaccaccaagggcatcctcaacgtcgccgtggagggcgagctgtggcggctgcgcggcaccgacggcaaggcgccgggcatgatcgggtggtggccagccaaggcggtgaccttcgtcgacaaccatgacaccggctccacgcagcatatgtggcccttcccttccgacagtgtcatgcagggatacgcgtacatcctcacgcacccagggaccccgtgcatcttctacgatcatttcttcgactggggcatgaaggacgagatctatcgcctagtgtcaatcaggacccggcaggggatacacagtgagagcaagctacaaatcatggaggctgacgccgacctttaccttgccgagatcgacggcaagatcatcgtcaagctcgggccacgattcgatgtggggcacctcattcccggaggcttcaaggtggccgcacacggcaaagactatgccgtatgggagaaaatataa

SEQ ID NO: 40 - protein, 427 aa, unidentified
  source 1..427: note = Triticum aestivum L.
MANKHPSLSLFLLLLGLSSSLASGQVLFQGFNWESWKHNGGWYNFLMGKVDDIAAAGVTHVWLPPASQSVSKQGYMPGRLYDLDASKYGNKAQLKSLIGALHGKGVKAIADIVINHRTAERKDGRGIYCIFEGGTPDARLDWGPHMICRDDRPYADGTGNPDTGADFGAAPDIDHLNPRVQKELVEWLNWLRTDVGFDGWRFDFAKGYSADVAKIYIDRSEASFAVAEIWTSLAYGGDGKPNLNQDPHRQELVNWVNKVGGSGTGTTFDFTTKGILNVAVEGELWRLRGTDGKAPGMIGWWPAKAVTFVDNHDTGSTQHMWPFPSDSVMQGYAYILTHPGTPCIFYDHFFDWGMKDEIYRLVSIRTRQGIHSESKLQIMEADADLYLAEIDGKIIVKLGPRFDVGHLIPGGFKVAAHGKDYAVWEKI

SEQ ID NO: 41 - DNA, 3179 bp, unidentified
  source 1..3179: mol_type = unassigned DNA; note = Triticum aestivum L.
cagaaaaaggtttgaagctcgctagaaagtttctgaacctggagtccagttttatctgcatcgagagatgcaccacatccgggttattcagggtaataatttgctgactggatttctgtttgttaaccaggaatgcgcaagtaacctcggagccatcctgcccacttgccaccagttgccgctgatcttttacgaagggttacggacagcatgtctggactggacctcattttagtgcgcgaaagctgaaaccatcggttgataagctgaagaactgtactggtttccatgttcttgccaattttcttggcttggatgctgttgctgttgcagtagatggtgatttgtaaccacagcactgttccatgaaaaatgctcaaatgttcttcatcaaagttcagaggttcagaataactgacagtcgttttgtccggtgccttcttaccgaaggcgaagactggctccatcacttgggccattgaattgccttttgagctcatcgcaccggccgataacaaactccggccgacatatccactggcccaaaggagcattcaagccgagcacgccgcaaaatgatttgcaagttgcaatcccggcatggtgcagcacagtataaatacctggccagacacaccagctgaatgcatcagttctccatcgtcctcttccaaagcacaggcagagctgaagaacatggcgaacaaacacttgtccctctcgctcttcctcgtcctccttggcctgtcggccaacttggcctccgggcaagttctctttcaggtaagatcctgttcgtcggcaagaactgtcttcagactgcgatcatgttttcttttggactggcctcttttggtttgtaggaatcttataggaggattttcaaggaaaaattcctatagagcccctttggtttgaaggaatgaattcctattgctatgtaggatagggtccaatccttcacatttgaaaggaaaaaaaacattagcctagattcagtgaaaaaaattcctatcctatgcattaaatggcatctctttttttataggaattcagatgcatgtcatttcactttttatgaagtttctattcctacaatattcttatcctatgaaccaaagaaggccggaactttgagttgtgcgtgcggcagggtttcaactgggagtcgtggaagcacaatggcgggtggtacaacttcctgatgggcaaggtggacgacatcgccgccgccggcgtcacgcacgtctggctccctccagcgtcgcagtccgtcgccgagcaaggctacatgccgggccggctgtacgacctggacgcctccaagtacggcaacaaggcgcagctcaagtccctcatcggggcgctccacggcaagggtgtcaaggccatcgccgacatcgtcatcaaccaccgcaccgcggagcacaaggacggccggggcatctactgcatcttcgagggcggcacccccgacgcccgccttgactggggcccccacatgatctgccgcgacgaccggccctacgccgacggcaccggcaacccggacactggcgccgacttcggggccgcccccgacatcgaccaccttaacccgcgcgtccagaaggagctcgtcgagtggctcaactggctcaaggccgacatcggcttcgacggctggcgcttcgacttcgccaagggctactccgcggacgtcgccaagatttacatcgaccgctcggcgcccagcttcgccgtggccgagatatggacgtcgctcgcgtacggcgtggacggcaagcccaacctcaaccaggaccaacaccggcaggagctggtgaactgggtggacaaggttggcggcaaaggccccgctaccacctttgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggctccacgcagcacatgtggcccttcccgtctgacagggtcatgcagggatatgcctacatcctgacgcacccagggaccccatgcatcgtgagtcatcgtaccaattcatcatatctaaattttcttctctttttttcgttcataataaaaaaccacgaccgaaatgatgaaaataatcgtgattctccagttctacgatcatttcttcgactggggccttaaggaggagatcgatcgtctggtgtcaatcaggacccgacatgggatacacagtgagagcaagctgcaaatcatagaggctgactctgacctttatctcgctgagatcgacggcaaggtcatcgtcaagctcgggccaagatacgatgtgggacacctcattccgggaggcttcaaggtggccgcgcatggcaatgactatgcggtgtgggagaaaatatgagcaaaattaccagagcagctctacgagttttttacaatatgaatgacatacatattagtccgagctcatgttgtccacatagtacgatttagtacttcctccatgtaaaagtgaggatgagggacatccattgtatttttaataaaataataatcaataaggatttttgcactcttggcttactttgtgttggttgaatgaaaaccgagcagccacgaccggtaactcgtacggctgcataccggagtagtggtagactcatgagcgttttggtacggaattgtgttacgggggctgggtttaatggagacaaactaatagtctagttactgaaaatttatttttgttcagtcactttttcatctgccgttaaattaagattaaacgtatacaaaacaatcatatacaacaaaaagttgactggccaaaattttggttcagtcgaattcctcacacaacctaactcatgcattttttttatttgcacacaaaaacgacgtgaggcccaaaaacaccagttgggctgatgcagcctacaagacggtaacccataacaacataaaaaacaacacttgttgtcattgttagactgcgtactggtacgttaacaaggcttaacctggtgcgcatggatggagctagatgagtatatacgcacatctgttaaaccatatggtttagcaaacatatgattaaatttgtacataggtgctccaaaaacgagta

SEQ ID NO: 42 - DNA, 1559 bp, unidentified
  source 1..1559: mol_type = unassigned DNA; note = Triticum aestivum L.
atggtgcagcacagtataaatacctggccagacacaccagctgaatgcatcagttctccatcgtcctcttccaaagcacaggcagagctgaagaacatggcgaacaaacacttgtccctctcgctcttcctcgtcctccttggcctgtcggccaacttggcctccgggcaagttctctttcagggtttcaactgggagtcgtggaagcacaatggcgggtggtacaacttcctgatgggcaaggtggacgacatcgccgccgccggcgtcacgcacgtctggctccctccagcgtcgcagtccgtcgccgagcaaggctacatgccgggccggctgtacgacctggacgcctccaagtacggcaacaaggcgcagctcaagtccctcatcggggcgctccacggcaagggtgtcaaggccatcgccgacatcgtcatcaaccaccgcaccgcggagcacaaggacggccggggcatctactgcatcttcgagggcggcacccccgacgcccgccttgactggggcccccacatgatctgccgcgacgaccggccctacgccgacggcaccggcaacccggacactggcgccgacttcggggccgcccccgacatcgaccaccttaacccgcgcgtccagaaggagctcgtcgagtggctcaactggctcaaggccgacatcggcttcgacggctggcgcttcgacttcgccaagggctactccgcggacgtcgccaagatttacatcgaccgctcggcgcccagcttcgccgtggccgagatatggacgtcgctcgcgtacggcgtggacggcaagcccaacctcaaccaggaccaacaccggcaggagctggtgaactgggtggacaaggttggcggcaaaggccccgctaccacctttgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggctccacgcagcacatgtggcccttcccgtctgacagggtcatgcagggatatgcctacatcctgacgcacccagggaccccatgcatcttctacgatcatttcttcgactggggccttaaggaggagatcgatcgtctggtgtcaatcaggacccgacatgggatacacagtgagagcaagctgcaaatcatagaggctgactctgacctttatctcgctgagatcgacggcaaggtcatcgtcaagctcgggccaagatacgatgtgggacacctcattccgggaggcttcaaggtggccgcgcatggcaatgactatgcggtgtgggagaaaatatgagcaaaattaccagagcagctctacgagttttttacaatatgaatgacatacatattagtccgagctcatgttgtccacatagtacgatttagtacttcctccatgtaaaagtgaggatgagggacatccattgtatttttaataaaataataatcaataaggatttttgcactcttggc

SEQ ID NO: 43 - DNA, 1380 bp, unidentified
  source 1..1380: mol_type = unassigned DNA; note = Triticum aestivum L.
atggtgcagcacagtataaatacctggccagacacaccagctgaatgcatcagttctccatcgtcctcttccaaagcacaggcagagctgaagaacatggcgaacaaacacttgtccctctcgctcttcctcgtcctccttggcctgtcggccaacttggcctccgggcaagttctctttcagggtttcaactgggagtcgtggaagcacaatggcgggtggtacaacttcctgatgggcaaggtggacgacatcgccgccgccggcgtcacgcacgtctggctccctccagcgtcgcagtccgtcgccgagcaaggctacatgccgggccggctgtacgacctggacgcctccaagtacggcaacaaggcgcagctcaagtccctcatcggggcgctccacggcaagggtgtcaaggccatcgccgacatcgtcatcaaccaccgcaccgcggagcacaaggacggccggggcatctactgcatcttcgagggcggcacccccgacgcccgccttgactggggcccccacatgatctgccgcgacgaccggccctacgccgacggcaccggcaacccggacactggcgccgacttcggggccgcccccgacatcgaccaccttaacccgcgcgtccagaaggagctcgtcgagtggctcaactggctcaaggccgacatcggcttcgacggctggcgcttcgacttcgccaagggctactccgcggacgtcgccaagatttacatcgaccgctcggcgcccagcttcgccgtggccgagatatggacgtcgctcgcgtacggcgtggacggcaagcccaacctcaaccaggaccaacaccggcaggagctggtgaactgggtggacaaggttggcggcaaaggccccgctaccacctttgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggctccacgcagcacatgtggcccttcccgtctgacagggtcatgcagggatatgcctacatcctgacgcacccagggaccccatgcatcttctacgatcatttcttcgactggggccttaaggaggagatcgatcgtctggtgtcaatcaggacccgacatgggatacacagtgagagcaagctgcaaatcatagaggctgactctgacctttatctcgctgagatcgacggcaaggtcatcgtcaagctcgggccaagatacgatgtgggacacctcattccgggaggcttcaaggtggccgcgcatggcaatgactatgcggtgtgggagaaaatatga

SEQ ID NO: 44 - protein, 459 aa, unidentified
  source 1..459: note = Triticum aestivum L.
MVQHSINTWPDTPAECISSPSSSSKAQAELKNMANKHLSLSLFLVLLGLSANLASGQVLFQGFNWESWKHNGGWYNFLMGKVDDIAAAGVTHVWLPPASQSVAEQGYMPGRLYDLDASKYGNKAQLKSLIGALHGKGVKAIADIVINHRTAEHKDGRGIYCIFEGGTPDARLDWGPHMICRDDRPYADGTGNPDTGADFGAAPDIDHLNPRVQKELVEWLNWLKADIGFDGWRFDFAKGYSADVAKIYIDRSAPSFAVAEIWTSLAYGVDGKPNLNQDQHRQELVNWVDKVGGKGPATTFDFTTKGILNVAVEGELWRLRGTDGKAPGMIGWWPAKAVTFVDNHDTGSTQHMWPFPSDRVMQGYAYILTHPGTPCIFYDHFFDWGLKEEIDRLVSIRTRHGIHSESKLQIIEADSDLYLAEIDGKVIVKLGPRYDVGHLIPGGFKVAAHGNDYAVWEKI

SEQ ID NO: 45 - DNA, 20 bp, unidentified
  source 1..20: mol_type = unassigned DNA; note = TaAmy1_A1_F1
ctcaactggctcaggaccaa

SEQ ID NO: 46 - DNA, 20 bp, unidentified
  source 1..20: mol_type = unassigned DNA; note = TaAmy1_genABD_R1
ccattgtgcttccacgactc

SEQ ID NO: 47 - DNA, 20 bp, unidentified
  source 1..20: mol_type = unassigned DNA; note = Amy1_B1I1_F1
gtaggtgccgcgatcatgta

SEQ ID NO: 48 - DNA, 20 bp, unidentified
  source 1..20: mol_type = unassigned DNA; note = Amy1_Bmix50I1_F1
agctcctatacgtaccgcga

SEQ ID NO: 49 - DNA, 20 bp, unidentified
  source 1..20: mol_type = unassigned DNA; note = Amy1_D1I1_F1
aagatattgtccgccggcaa

SEQ ID NO: 50 - DNA, 20 bp, unidentified
  source 1..20: mol_type = unassigned DNA; note = Amy1_ABDI1_R
actcccagttgaaaccctgc

SEQ ID NO: 51 - DNA, 1757 bp, unidentified
  source 1..1757: mol_type = unassigned DNA; note = Triticum aestivum
atggcgaacaaacacttgtccctctcgctcttcctcgtcctccttggcctgtcggccaacttggcctccgggcaagttctctttcaggtaagatcctgttcgtcggcaagaactgtcttcagactgcgatcatgttttcttttggactggcctcttttggtttgtaggaatcttataggaggattttcaaggaaaaattcctatagagcccctttggtttgaaggaatgaattcctattgctatgtaggatagggtccaatccttcacatttgaaaggaaaaaaaacattagcctagattcagtgaaaaaaattcctatcctatgcattaaatggcatctctttttttataggaattcagatgcatgtcatttcactttttatgaagtttctattcctacaatattcttatcctatgaaccaaagaaggccggaactttgagttgtgcgtgcggcagggtttcaactgggagtcgtggaagcacaatggcgggtggtacaacttcctgatgggcaaggtggacgacatcgccgccgccggcgtcacgcacgtctggctccctccagcgtcgcagtccgtcgccgagcaaggctacatgccgggccggctgtacgacctggacgcctccaagtacggcaacaaggcgcagctcaagtccctcatcggggcgctccacggcaagggtgtcaaggccatcgccgacatcgtcatcaaccaccgcaccgcggagcacaaggacggccggggcatctactgcatcttcgagggcggcacccccgacgcccgccttgactggggcccccacatgatctgccgcgacgaccggccctacgccgacggcaccggcaacccggacactggcgccgacttcggggccgcccccgacatcgaccaccttaacccgcgcgtccagaaggagctcgtcgagtggctcaactggctcaaggccgacatcggcttcgacggctggcgcttcgacttcgccaagggctactccgcggacgtcgccaagatttacatcgaccgctcggcgcccagcttcgccgtggccgagatatggacgtcgctcgcgtacggcgtggacggcaagcccaacctcaaccaggaccaacaccggcaggagctggtgaactgggtggacaaggttggcggcaaaggccccgctaccacctttgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggctccacgcagcacatgtggcccttcccgtctgacagggtcatgcagggatatgcctacatcctgacgcacccagggaccccatgcatcgtgagtcatcgtaccaattcatcatatctaaattttcttctctttttttcgttcataataaaaaaccacgaccgaaatgatgaaaataatcgtgattctccagttctacgatcatttcttcgactggggccttaaggaggagatcgatcgtctggtgtcaatcaggacccgacatgggatacacagtgagagcaagctgcaaatcatagaggctgactctgacctttatctcgctgagatcgacggcaaggtcatcgtcaagctcgggccaagatacgatgtgggacacctcattccgggaggcttcaaggtggccgcgcatggcaatgactatgcggtgtgggagaaaatatga

SEQ ID NO: 52 - DNA, 1478 bp, unidentified
  source 1..1478: mol_type = unassigned DNA; note = Triticum aestivum
atggcgaacaaacactcgtccctccccctcttcctcgtcctccttggcctgtcggccagcttggcctccggtcaaattctctttcaggtattatcttcttcgtcggcaagaactgtcttcaggctgcgatcatgttttgttttggactgagttttgagttctgctgcgtgcggcagggtttcaactgggagtcgtggaagcacaatggcgggtggtacaacttcctgatgggcaaggtggacgacatcgccgccggcggcatcacgcacgtgtggctccccccggcgtcgcagtccgtggccgagcaaggctacatgccgggccggctgtacgacctggacgcctccaagtacggcaacaaggcgcagctcaagtccctgatcggggcgctccacggcaagggcgtcaaggccatcgccgacattgtcatcaaccaccgcacggcggagcgcaaggacggccggggcatctactgcatcttcgagggcggcacccccgacgcgcgccttgactggggcccccacatgatctgccgcgacgaccggccctacgccgacggcacgggcaacccggacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctccgaggccagcttcgccgtggccgagatatggacgtcgctggcgtacggcggggacggcaagcccaacctcaaccaggacccgcaccggcaggagctggtgaactgggtgaacaaggtgggcggctccggccccggcaccacgttcgacttcaccaccaagggcatcctcaatgtggccgtggagggcgagctgtggcggctgcgcggcactgacggcaaggcgccgggcatgatcgggtggtggccagccaaggcagtgaccttcgtggacaaccacgacaccggctccacgcaacacatgtggcccttcccttctgacagggtcatgcagggatatgcctacatcctcacgcacccagggaccccatgcatcgtgagtcatcgtgctattgccaactcatcatatctaaattgtccttttttctccgttcatatgaaaccatgaccaaactgatgaaaactaaatgtgattcttcagttctacgatcatttcttcgactggggcctgaaggaggagatcgatcgcctggtgtcaatcaggacccgacaggggatacacagtgagagcaagctacaaatcatagaggctgacgccgacctttacctagccgagatcgacagcaaggtcatcgtcaaactcgggccaagatacgatgtcgggcacctcattcccggaggcttcaaggtggtcgcgcacggcaaagattatgccgtatgggagaaaatataa

SEQ ID NO: 53 - DNA, 1478 bp, unidentified; identical to SEQ ID NO: 52
  source 1..1478: mol_type = unassigned DNA; note = Triticum aestivum

SEQ ID NO: 54 - DNA, 1468 bp, unidentified
  source 1..1468: mol_type = unassigned DNA; note = Triticum aestivum
atggcgaacaaacactcgtccctccccctcttcctcgtcctccttggcctgtcggccagcttggcctccgggcaactcctgtttcaggtaagaataagatcttgtacccgtcggcaagctcctgggttttggaccgagttttgacttctgctgagtgcggcagggtttcaactgggagtcgtggaagcacaacggcgggtggtacaacttcctgatgggcaaggtggacgacatcgccgcggccgggatcacgcacgtctggctccctccggcgtcgcagtccgtcgccgagcaagggtacatgccgggccggctctacgacctggacgcgtccaagtacggcaacaaagcgcagctcaagtccctcatcggggcgctccacggcaagggcgtcaaggccatcgccgacatcgtcatcaaccaccgcacggcggagcgcaaggacggccggggcatctactgcatcttcgagggcggcaccccggacgcgcgcctcgactggggcccccacatgatctgccgcgacgaccggccctacgccgacggcacgggcaacccggacaccggcgccgacttcggggccgccccggacatcgaccacctcaacccgcgcgtccagaaggagctcgtcgagtggctcaactggctcaggaccgacgtcggcttcgacggctggcgcttcgacttcgccaagggctactcggcggacgtcgccaagatctacatcgaccgctccgaggccagcttcgccgtggccgagatatggacgtcgctggcgtacggcggggacggcaagccgaacctcaaccaggaccagcaccggcaggagctggtgaactgggtgaacaaggtgggcggctccggcccgggcaccacgttcgacttcaccaccaagggcatcctcaacgtcgccgtggagggcgagctgtggcggctgcgcggcacagacggcaaggcgccaggcatgatcgggtggtggccagccaaggcggtgaccttcgtcgacaaccatgacaccggctccacgcagcacatgtggcccttcccttccgacagggtcatgcagggatacgcctacatcctcacgcacccagggaccccatgcatcgtgagtcatcctaccagttcatcgtatctaaattgctcttcttcctttttcgttcatataagaaaccatggatgaccgaactgatgaaaaataattgtgattcttcagttctacgatcatttcttcgactggggcctaaaggaggagatcgatcgcctagtgtcaatcaggaccaggcagggcatacacagtgagagcaagctgcaaatcatagaggctgacgccaatctttaccttgccgagatcgacggcaaggtcatcgtcaagctcgggccaagatacgatgtggggcacctcattcccggaggctttaaggtggccgcacacggcaaagactatgctgtatgggagaaaatataa

SEQ ID NO: 55 - DNA, 1494 bp, unidentified
  source 1..1494: mol_type = unassigned DNA; note = Triticum aestivum
atggcgaacaaacacttgtccctcttcctcgtcctccttggcctgtcggccagcttggcatccgggcaagtcctctttcaggtaagatcgtgtctgttgtctgtaactgtaagcattatcttcagatcgtaggtgccgcgatcatgtatgttttggaccgagttttgatccgagttctgtggtagggcttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcaccgcggagcacaaggacggccggggcatctactgcatcttcgagggcggcacccccgacgcccgcctcgactggggcccccacatgatctgccgcgacgaccggccctacgccgacggcaccggcaacccggacaccggcgccgacttcggggccgcccccgacatcgaccacctcaacccgcgcgtccagaaggagctcgtcgagtggctcaactggctcaggaccgacgtcggcttcgacggctggcgcttcgacttcgccaagggctactccgcggacgtcgccaagatttacattgaccgctcggagcccagcttcgccgtggccgagatatggacgtcgctcgcgtacagcggtgacggcaagccgaacctcaaccaggaccagcaccggcaggagctggtgaactgggtggacaaggttggcggcaaaggccccgctaccacgttcgacttcaccaccaagggcatcctgaacgtggccgtggagggcgagctgtggcggctgcgcggcaccgacggcaaggcgccaggcatgatcgggtggtggcccgccaaggcggtgacctttgtggacaaccacgacaccggctccacgcagcacatgtggcccttcccgtctgacatggtcatgcagggatatgcctacatcctcacgcatccaggcaccccatgcatcgtgagtcatcctaccaatactacatcatatctaaattttcttttcttctttcgttcataaaaatataagaaaaccatgacccaactgatgaaaattaattgtgactcttcagttctacgatcatttcttcgactggggcctgaaggacgagatcgatcgcctggtgtcaatcaggacccgacatgggatacacagtgaaagcaagctgcaaatcatagaggccgacgccgatctttatctcgccgagatcgatggcaaggtcatcgtcaagctcgggccaagatacaatgtgggacacctcattccgggaggcttcaaggtggccgcacacggcaatgactatgccgtatgggagaaaatatga

SEQ ID NO: 56 - DNA, 1773 bp, unidentified
  source 1..1773: mol_type = unassigned DNA; note = Triticum aestivum
atggcgaacaaacacttgtccctctcgctcttcctcgtcctccttggcctgtcggccagcttggcctccgggcaagtcctgtttcaggtaagctcctgttcgtcggcaagaactgtcttcagactgtgatcatgttttcttttggactgaggcctcctttggtttgtaggaatctcatatgaatttcatagaataggatttttaaatgaaaaattcctatggagcccttcagtttgtacgaataaatttctattactgtgtatgatagggttcaatccttcacatttcaaagaaaaaaaatattaatctagactcaatgaaaaaattcctatcttatgcattaaatgacatctctttctttatagaaattcagatgcatgtcatctcacttcatatgaagttcctattcctataatattcctatcctatgaaccaaagagggcctcaactttgagttgtgcgtgcggcagggtttcaactgggagtcgtggaagcacaatggcgggtggtacaacttcctgatgggcaaggtggacgacatcgccgccgccggcatcacgcacgtctggctccctccggcgtcgcagtccgtcgccgagcaaggctacatgccgggccggctctacgacctggacgcctccaagtacggcaacaaggcgcagctcaagtccctcatcggggcgctccacggcaagggcgtcaaggccatcgccgacatcgtcatcaaccaccgcacggcggagcacaaggacggccggggcatctactgcatcttcgaaggcggcacccccgacgcccgcctcgactggggcccccacatgatctgccgtgacgaccggccctacgccgacggcaccggcaacccggacaccggcgccgacttcggggccgcccccgacatcgaccacctcaacccgcgcgtccagaaggagctcgtcgagtggctcaactggctcaagaccgacatcggcttcgacggatggcgcttcgacttcgccaagggctactccgcggacgtcgccaagatttacatcgaccgctcggagcccggcttcgccgtggccgagatatggacgtcgctggcgtacggcggtgacggcaagcccaacctgaaccaggaccagcaccggcaggagctggtgaactgggtggacaaggtcggcggcaaaggccccgctaccacgttcgacttcaccaccaagggcatcctgaacgtcgccgtggagggcgagctgtggcggctgcgcggcaccgacggcaaggcgccaggcatgatcgggtggtggccagccaaggcggtgaccttcgtggacaaccacgacaccggatccacgcagcacatgtggcccttcccttctgacagggtcatgcagggatatgcctacatcctcacgcacccagggaccccatgcatcgtgagtcatcgtaccaatactacatcatatctaaattttcttctcgttttttcgttcattatagaaaaccacgaccgaactgatgaaaataatcgtgattctccagttctacgatcatttcttcgattggggccttaaggaggagatcgatcgcctggtgtcaatcaggacccggcatgggatacacagtgagagcaagctgcaaatcatagaggccgacgccgacctttatctcgccgagatcgacggcaaggtcatcgtcaagctcgggccaagatacgatgtggggcacctcattccgggaggcttcaaggtgggcgcgcacggcaatgactatgccgtatgggagaaaatatga

SEQ ID NO: 57 - DNA, 1473 bp, unidentified
  source 1..1473: mol_type = unassigned DNA; note = Triticum aestivum
atggcgaacaaacactcgtccctctccctcttcctcgtcctccttggcctgtcggcaagcttagcctccggtcaagtcctgtttcaggtaagatcatgtcctgtcttcaacttctacacgtactgtgatcatgtttcggggactgagttttcagttctgcatgcggcagggtttcaactgggagtcgtggaagcacaatggcgggtggtacaacttcctgatgggcaaggtggacgacatcgccgccgccggcgtcacgcacgtgtggctccccccggcgtcgcagtccgtcgccgagcaagggtacatgccgggccggctgtacgacctggacgcgtccaagtacggcaacaaggcgcagctcaagtccctgatcggggcgctccacggcaagggcgtcaaggccatcgccgacatcgtcatcaaccaccgcacggcggagcgcaaggacggccggggcatctactgcatcttcgagggcggcacccctgacgcgcgcctcgactggggcccccacatgatctgccgcgacgaccggccctacgccgacggcacgggcaacccagacaccggcgccgacttcggggccgccccggacatcgaccacctcaacccgcgcgtccagaaggagctcgtggagtggctcaactggctcaggaccgacgtcggcttcgacggctggcgcttcgacttcgccaagggctactccgcggacgtcgccaagatctacatcgaccgctcggagcccagcttcgccgtggccgagatatggacgtcgctggcgtacggcggggacggcaagcccaacctcaaccaggaccagcaccggcaggagctggtgaactgggtgaacaaggtgggcggctccggccccggcaccacgttcgacttcaccaccaaaggcatcctcaacgtggctgtggagggcgagctgtggcggctgcgcggcaccgacggcaaggcgccaggcatgatcgggtggtggccagccaaggcggtgaccttcgtggacaaccatgacaccggctccacgcagcacatgtggcccttcccttccgacaaggtcatgcagggatatgcctacatcctcacgcaccctgggaccccatgcatcgtgagtcatcgtgccattgccaaatcatcatatctaaattgtctttttttttcgtccgttcataagaaaccatgacctaacttgacaaaaaaaatgatattcttcagttctacgatcatttcttcgactgggggctgaaggaggagatcgatcgcctggtgtcaatcaggacccggcaggggatacacagtgagagcaagctgcaaatcatagaggccgacgccgacctttacctggccgagatcgatggcaaggtcatcgtcaagctcgggccaagatacgatgtcgggcacctcattcctcaaggcttcaaggtggtcgcgcacggcaatgactatgccgtatgggagaaaatataa

SEQ ID NO: 58 - DNA, 1470 bp, unidentified
  source 1..1470: mol_type = unassigned DNA; note = Triticum aestivum
atggcgagcaaacactcgtccctctccctcttcctcgtcctccttggcctgtcggccagcttggcctccgggcaagtcctgtttcaggtaagatcgtgccctgtcttcagcttctacacgtactactgtgatcatgtttcggggactgagctttgagttctgctgcgtgcggcagggtttcaactgggagtcgtggaagcacaatggcgggtggtacaacttcctgatgggcaaggtggacgacatcgccgccgccggcgtcacgcacgtctggctccctccggcgtcgcagtccgtcgccgagcaagggtacatgcctggccggctctacgacctggacgcctccaagtacggcaacaaggcgcagctcaagtccctcatcggggcgctccacggcaagggcgtcaaggccatcgccgacatcgtcatcaaccaccgcacggcggagcgcaaggacgggcggggcatctactgcatcttcgagggcggcaccccggacgcgcgcctcgactggggcccccacatgatctgccgcgacgatcggccctacgccgacggcaccggcaacccagacacgggcgccgacttcggggccgccccggacatcgaccacctcaacccgcgtgtgcagaaggagctcgtcgagtggctcaactggctcaggaccaacgtcggcttcgacggctggcgcttcgacttcgccaagggctactccgcggacgtcgccaagatctacatcgaccgctccggggccagcttcgccgtggccgagatatggacgtcgctggcgtacggtggggacggcaagccgaacctcaaccaggacccgcaccggcaggagctggtgaactgggtgaacaaggtgggcggctccggccccggcaccacgttcgacttcaccaccaagggcatcctcaacgtggccgtggagggcgagctctggcggctgcgcggcaccgacggcaaggcgccaggcatgatcgggtggtggccagccaaggcggtgaccttcgtggacaaccacgacaccggctccacgcagcacatgtggcccttcccttctgacagggtcatgcagggatacgcctacatcctcacgcaccctgggaccccatgcatcgtgagtcatcctaccagttcatcgtctaaattgctcttcttttttcgttcatataagaacccatgcatgagtgacgaaaaatattcgtgattcttcagttctacgatcatttcttcgactggggcctgaaggaggagatcgatcgcctggtgtcaatcaggacccggcaggggatacacagtgagagcaagctgcaaatcatagaggctgacgccgacctttacctggctgagatcgacggcaaggtcatcgtcaagctcgggccaagatacgatgtcgggcacctcattccccaaggcttcaaggtggtcgcgcacggcaatgactatgccgtatgggagaaaatataa

SEQ ID NO: 59 - protein, 13 aa, synthetic construct
  REGION 1..13: note = Reduced and alkylated digested peptide
QVLFQGFNWESWK

SEQ ID NO: 60 - protein, 30 aa, synthetic construct
  REGION 1..30: note = Reduced and alkylated digested peptide
SEPSFAVAEIWTSLAYGGDGKPNLNQDQHR

SEQ ID NO: 61 - protein, 23 aa, synthetic construct
  REGION 1..23: note = Reduced and alkylated digested peptide
AVTFVDNHDTGSTQHMWPFPSDK

SEQ ID NO: 62 - protein, 30 aa, synthetic construct
  REGION 1..30: note = Reduced and alkylated digested peptide
VDDIAAAGVTHVWLPPASQSVAEQGYMPGR

SEQ ID NO: 63 - protein, 8 aa, synthetic construct
  REGION 1..8: note = Reduced and alkylated digested peptide
LYDLDASK

SEQ ID NO: 64 - protein, 9 aa, synthetic construct
  REGION 1..9: note = Reduced and alkylated digested peptide
SLIGALHGK

SEQ ID NO: 65 - protein, 10 aa, synthetic construct
  REGION 1..10: note = Reduced and alkylated digested peptide
AIADIVINHR

SEQ ID NO: 66 - protein, 14 aa, synthetic construct
  REGION 1..14: note = Reduced and alkylated digested peptide
GIYCIFEGGTPDAR

SEQ ID NO: 67 - protein, 30 aa, synthetic construct
  REGION 1..30: note = Reduced and alkylated digested peptide
DDRPYADGTGNPDTGADFGAAPDIDHLNPR

SEQ ID NO: 68 - protein, 10 aa, synthetic construct
  REGION 1..10: note = Reduced and alkylated digested peptide
ELVEWLNWLR

SEQ ID NO: 69 - protein, 8 aa, synthetic construct
  REGION 1..8: note = Reduced and alkylated digested peptide
GYSADVAK

SEQ ID NO: 70 - protein, 9 aa, synthetic construct
  REGION 1..9: note = Reduced and alkylated digested peptide
QELVNWVNK

SEQ ID NO: 71 - protein, 15 aa, synthetic construct
  REGION 1..15: note = Reduced and alkylated digested peptide
VGGSGPGTTFDFTTK

SEQ ID NO: 72 - protein, 13 aa, synthetic construct
  REGION 1..13: note = Reduced and alkylated digested peptide
GILNVAVEGELWR

SEQ ID NO: 73 - protein, 11 aa, synthetic construct
  REGION 1..11: note = Reduced and alkylated digested peptide
APGMIGWWPAK

SEQ ID NO: 74 - protein, 23 aa, synthetic construct
  REGION 1..23: note = Reduced and alkylated digested peptide
AVTFVDNHDTGSTQHMWPFPSDR

SEQ ID NO: 75 - protein, 18 aa, synthetic construct
  REGION 1..18: note = Reduced and alkylated digested peptide
LQIIEADADLYLAEIDGK

SEQ ID NO: 76 - protein, 12 aa, synthetic construct
  REGION 1..12: note = Reduced and alkylated digested peptide
YDVGHLIPQGFK

SEQ ID NO: 77 - protein, 13 aa, synthetic construct
  REGION 1..13: note = Reduced and alkylated digested peptide
VVAHGNDYAVWEK

SEQ ID NO: 78 - protein, 13 aa, synthetic construct
  REGION 1..13: note = Reduced and alkylated digested peptide
VGAHGNDYAVWEK

SEQ ID NO: 79 - protein, 30 aa, synthetic construct
  REGION 1..30: note = Reduced and alkylated digested peptide
VDDIAAAGVTHVWLPPASQSVSEQGYMPGR

SEQ ID NO: 80 - protein, 9 aa, synthetic construct
  REGION 1..9: note = Reduced and alkylated digested peptide
TDIGFDGWR

SEQ ID NO: 81 - protein, 30 aa, synthetic construct
  REGION 1..30: note = Reduced and alkylated digested peptide
VDDIAAAGITHVWLPPASQSVAEQGYMPGR

SEQ ID NO: 82 - protein, 23 aa, synthetic construct
  REGION 1..23: note = Reduced and alkylated digested peptide
VDDIAAAGVTHVWLPPASQSVSK

SEQ ID NO: 83 - protein, 30 aa, synthetic construct
  REGION 1..30: note = Reduced and alkylated digested peptide
SEASFAVAEIWTSLAYGGDGKPNLNQDPHR

SEQ ID NO: 84 - protein, 12 aa, synthetic construct
  REGION 1..12: note = Reduced and alkylated digested peptide
FDVGHLIPGGFK

SEQ ID NO: 85 - protein, 7 aa, synthetic construct
  REGION 1..7: note = Reduced and alkylated digested peptide
DYAVWEK

SEQ ID NO: 86 - protein, 23 aa, synthetic construct
  REGION 1..23: note = Reduced and alkylated digested peptide
AVTFVDNHDTGSTQHMWPFPSDR

SEQ ID NO: 87 - protein, 10 aa, synthetic construct
  REGION 1..10: note = Reduced and alkylated digested peptide
ELVEWLNWLK

SEQ ID NO: 88 - protein, 9 aa, synthetic construct
  REGION 1..9: note = Reduced and alkylated digested peptide
ADIGFDGWR

SEQ ID NO: 89 - protein, 9 aa, synthetic construct
  REGION 1..9: note = Reduced and alkylated digested peptide
QELVNWVDK

SEQ ID NO: 90 - protein, 11 aa, synthetic construct
  REGION 1..11: note = Reduced and alkylated digested peptide
GPATTFDFTTK

SEQ ID NO: 91 - protein, 18 aa, synthetic construct
  REGION 1..18: note = Reduced and alkylated digested peptide
LQIIEADSDLYLAEIDGK

SEQ ID NO: 92 - protein, 12 aa, synthetic construct
  REGION 1..12: note = Reduced and alkylated digested peptide
YDVGHLIPGGFK

SEQ ID NO: 93 - protein, 13 aa, synthetic construct
  REGION 1..13: note = Reduced and alkylated digested peptide
VAAHGNDYAVWEK

SEQ ID NO: 94 - protein, 13 aa, synthetic construct
  REGION 1..13: note = Reduced and alkylated digested peptide
GVQAIADIVINHR

SEQ ID NO: 95 - protein, 16 aa, synthetic construct
  REGION 1..16: note = Reduced and alkylated digested peptide
VGGAASAGMVFDFTTK

SEQ ID NO: 96 - protein, 14 aa, synthetic construct
  REGION 1..14: note = Reduced and alkylated digested peptide
GIYCIFEGGTSDGR

SEQ ID NO: 97 - protein, 9 aa, synthetic construct
  REGION 1..9: note = Reduced and alkylated digested peptide
SDLGFDAWR

SEQ ID NO: 98 - protein, 8 aa, synthetic construct
  REGION 1..8: note = Reduced and alkylated digested peptide
GYSPEMAK

SEQ ID NO: 99 - protein, 9 aa, synthetic construct
  REGION 1..9: note = Reduced and alkylated digested peptide
QNLVNWVDK

SEQ ID NO: 100 - protein, 13 aa, synthetic construct
  REGION 1..13: note = Reduced and alkylated digested peptide
GILNAAVEGELWR

SEQ ID NO: 101 - protein, 11 aa, synthetic construct
  REGION 1..11: note = Reduced and alkylated digested peptide
APGVMGWWPAK

SEQ ID NO: 102 - protein, 16 aa, synthetic construct
  REGION 1..16: note = Reduced and alkylated digested peptide
ILMHEGDAYVAEIDGK

SEQ ID NO: 103 - protein, 23 aa, synthetic construct
  REGION 1..23: note = Reduced and alkylated digested peptide
AVTFVDNHDTGSTQALWPFPSDK

(19) United States
(12) Patent Application Publication
(10) Pub. No.: US 2024/0349677 A1
(43) Pub. Date: Oct. 24, 2024

(54) WHEAT WITH REDUCED SUSCEPTIBILITY TO LATE-MATURITY ALPHA-AMYLASE

(71) Applicants: COMMONWEALTH SCIENTIFIC AND INDUSTRIAL RESEARCH ORGANISATION, Territory (AU); GRAINS RESEARCH AND DEVELOPMENT CORPORATION, Australian Capital Territoryy (AU)

(72) Inventors: Jean-Philippe RAL, Kaleen, Australian Capital Territory (AU); Crispin HOWITT, Kaleen, Australian Capital Territory (AU); Jos MEOG, Goonellabah, New South Wales (AU); Michelle COLGRAVE, Everton Hills, Queensland (AU); James BROADBENT, Alderley, Queensland (AU); Uptal BOSE, Kenmore, Queensland (AU); Steve JOBLING, Nicholls, Australian Capital Territory (AU)

(21) Appl. No.: 18/685,152

(22) PCT Filed: Aug. 18, 2022
(86) PCT No.: PCT/AU2022/050917

§ 371 Date: Feb. 20, 2024

(30) Foreign Application Priority Data
Aug. 18, 2021 (AU) 2021902585

(51) Int. Cl. A01H 6/46; A01H 1/00; A01H 5/10; C12N 9/32; C12Q 1/6895

(52) U.S. Cl. CPC A01H 6/4678; A01H 1/10; A01H 5/10; C12N 9/2422; C12Q 1/6895; C12Y 302/01001; C12Q 2600/156

(57) ABSTRACT

The present disclosure relates generally to wheat plants comprising a genetic modification leading to reduced expression of at least one Amy 1 gene on chromosome 6B. The present disclosure further relates to grain from such plants and to products derived from the grain.

### RELATED APPLICATIONS

This application claims priority from Australian Provisional Patent Application No. 2021902585 filed on 18 Aug. 2021, the entire content of which is hereby incorporated by reference.

### FIELD

The present disclosure relates generally to wheat plants comprising a genetic modification leading to reduced expression of at least one Amy1 gene on chromosome 6B. The present disclosure further relates to grain from such plants and to products derived from the grain.

### BACKGROUND

Wheat is the most widely cultivated crop on earth, contributing one-fifth of the total calories consumed by humans. However, high alpha-amylase (α-amylase) levels in harvested grain can reduce the quality and marketability of wheat.

Pre-harvest sprouting (PHS) and late maturity alpha-amylase (LMA) are the two major causes of increased expression of alpha-amylase in wheat grain. PHS is characterized by germination of the grain on the head before ripening induced by severe weather conditions, whereas LMA is associated with constitutive genetic defects and temperature changes that cause elevated alpha-amylase levels during grain development. Contamination by LMA or PHS corresponds to low Falling Number, an accepted measure for grain grading, which results in down-grading of the grain quality (e.g., feed grade), significantly reducing the income that can be derived from a harvest. It is estimated that as little as 5%-10% contamination of LMA or PHS is sufficient to lower the Falling Number below an acceptable level.

LMA has emerged as a key problem in the wheat industry, with the increasing global prevalence of LMA impeding the development of improved wheat varieties. The increased prevalence of LMA may be associated with changes in weather patterns, with recent studies indicating that LMA can be triggered by heat waves or hot spells during development (Barrero et al., 2020, Scientfic Reports, 10: 17800). Accordingly, it is expected that LMA will occur more frequently due to climate change leading to more unpredictable weather patterns, variation in rainfall and altered periods of winter or summer.

The development of early detection methods for LMA have been limited due to the phenotypic variation of plants affected by LMA. In particular, LMA triggered by environmental conditions results in variable expression between growing locations, between plants and between heads or spikelets of the same plant, with not affect grain morphology, composition or germination. As a result, detection of LMA using non-destructive phenomic phenotyping is considered too complicated to be practical for breeders or growers. Other existing methods for the detection of LMA are low-throughput and require sampling to occur towards the end of the breeding cycle (Mares and Panozzo, 1994, Australian Journal of Agricultural Research, 45(5): 1003-1011), which limits the ability of breeders to test and release new lines in a timely manner without incurring financial losses. Due to these limitations, strategies to mitigate or eliminate LMA have focused on understanding the genetic basis of LMA applying techniques used to identify quantitative trait loci (QTL) that control the expression of LMA. To date, however, only a few QTLs associated with LMA have been investigated, the majority of which are associated with genetic variation located on chromosome 7B (e.g., Mrva and Mares, 2001, Australian Journal of Agricultural Research, 52: 1267-1273 and Mares and Mrva, 2008, Journal of Cereal Science, 47: 6-17).

There remains, therefore, a need for improved wheat plants that have reduced susceptibility to the deleterious effects of LMA and methods for the early detection of such wheat plants.

### SUMMARY OF THE DISCLOSURE

In an aspect of the present disclosure there is provided a wheat plant comprising a genetic modification leading to reduced expression of at least one Amy1 gene on chromosome 6B relative to a wild-type wheat plant, wherein grain from the plant is characterized by a reduced level or activity, or both, of alpha-amylase 1 (AMY1) polypeptides relative to a wild-type wheat plant.

In another aspect, the present disclosure provides a process for producing wheat grain, comprising:
a. growing a wheat plant as disclosed herein, preferably in a field as part of a population of at least 1000 such plant or in an area of at least 1 hectare planted at a standard planting density; and
b. harvesting grain from the plant.

In another aspect, the present disclosure provides a process for producing bins of wheat grain, comprising:
a. reaping above-ground parts of wheat plants as disclosed herein;
b. threshing and/or winnowing the parts of the wheat plants to separate the grain from the remainder of the plant parts;
c. sifting and/or sorting the grain separated in step (b); and
d. loading the sifted and/or sorted grains into bins, thereby producing bins of grain.

In another aspect, the present disclosure provides wheat grain obtained from the wheat plants as disclosed herein, or produced by the process disclosed herein.

In another aspect, the present disclosure provides wheat grain comprising a genetic modification leading to reduced expression of at least one Amy1 gene on chromosome 6B relative to a wild-type wheat grain, wherein the grain is characterized by a reduced level or activity, or both, of AMY1 polypeptides relative to a wild-type wheat grain.

In another aspect, the present disclosure provides a wheat cell derived from the wheat plant or the wheat grain as disclosed herein.

In another aspect, the present disclosure provides a nucleic acid molecule which encodes an AMY1 polypeptide, wherein the nucleic acid molecule differs from the nucleic acid of SEQ ID NO: 13, 17, 21, 25 and/or 29, or a nucleic acid sequence which is at least 91% identical to the nucleic acid sequence of SEQ ID NO: 13, 17, 21, 25 and/or 29, by a mutation selected from a deletion mutation, an insertion mutation, a splice-site mutation, a premature translation, a termination mutation, a nonsense mutation and a frameshift mutation.

In another aspect, the present disclosure provides an AMY1 polypeptide, wherein the polypeptide differs from the amino acid sequence of SEQ ID NO: 16, 20, 24, 28 and/or 32, or an amino acid sequence which is at least 99% identical to the amino acid sequence of SEQ ID NO: 16, 20, 24, 28 and/or 32, by at least one amino acid substitution, insertion or deletion in the active site, catalytic site, calcium binding site and/or carbohydrate binding domain, wherein the AMY1 polypeptide has an impaired structure and/or reduced activity relative to an AMY1 polypeptide having the amino acid sequence of SEQ ID NO: 16, 20, 24, 28 and/or 32.

In another aspect, the present disclosure provides a method of producing wheat flour, wholemeal, starch, starch granules or bran, the method comprising obtaining the grain as disclosed herein and processing the grain to produce the flour, wholemeal, starch, starch granules or bran.

In another aspect, the present disclosure provides wheat flour, wholemeal, starch, starch granules or bran produced by the method as disclosed herein, or comprising the nucleic acid molecule or the polypeptide as disclosed herein.

In another aspect, the present disclosure provides a method of producing a food product, comprising mixing the grain, or the wheat flour, wholemeal, starch, starch granules or bran as disclosed herein with at least one other food ingredient to produce the food product.

In another aspect, the present disclosure provides a food product comprising the wheat grain, the wheat cell, the nucleic acid molecule, or the polypeptide as disclosed herein, or an ingredient which is the wheat flour, wholemeal, starch, starch granules or bran as disclosed herein.

In another aspect, the present disclosure provides a method of genotyping a wheat plant or grain, the method comprising:
a. obtaining a sample comprising nucleic acid extracted from a wheat plant or grain; and
b. detecting in the sample a nucleic acid molecule as disclosed herein.

In another aspect, the present disclosure provides a method of selecting a wheat plant from a population of wheat plants, wherein said population of plants comprises progeny plants obtained from a cross between two plants of which at least one plant was a wheat plant as disclosed herein, the method comprising:
a. genotyping each one or more progeny plants in said population of wheat plants using the method as disclosed herein; and
b. based on the results of the genotyping in step (a), selecting a progeny plant which comprises the nucleic acid molecule as disclosed herein.

In another aspect, the present disclosure provides a method for reducing susceptibility to LMA into a wheat plant, the method comprising:
a. crossing a first parent wheat plant with a second parent wheat plant, wherein the second plant is a wheat plant as disclosed herein; and
b. backcrossing a progeny plant of the cross of step (a) with a plant of the same genotype as the first parent plant to produce a plant with a majority of genotype of the first parent but comprising a nucleic acid molecule as disclosed herein.

In another aspect, the present disclosure provides a method of producing a plant with reduced susceptibility to LMA, the method comprising:
a. introducing a genetic modification to at least one Amy1 gene on chromosome 6B to a plant cell such that the cell has reduced level or activity, or both, of AMY1 polypeptides relative to unmodified cells;
b. regenerating a plant with the genetic modification from the cell of step (a), wherein the plant has reduced susceptibility to LMA relative to wild-type plants lacking the genetic modification.

In another aspect, the present disclosure provides a method of determining susceptibility to LMA in a wheat plant, the method comprising:
a. obtaining a sample of grain from the wheat plant;
b. analyzing the sample for the presence of at least one AMY1 peptide selected from the amino acid sequences of SEQ ID NOs: 59-93, or an amino acid sequence which comprises not more than three modifications to an amino acid sequence of SEQ ID NOs: 59-93; and/or at least one AMY2 peptide selected from the amino acid sequences of SEQ ID NOs: 94-103, or an amino acid sequence which comprises not more than three modifications to an amino acid sequence of SEQ ID NOs: 94-103;
c. comparing the peptides analyzed in step (b) to those present in control wheat grain, and based on this comparison, determining if the wheat plant is susceptible to LMA.

### BRIEF DESCRIPTION OF THE DRAWINGS

Embodiments of the disclosure are described herein, by way of non-limiting example only, with reference to the accompanying drawings.

FIG. 1 is a schematic representation of the distribution of Amy1 genes in wheat (modified from Ju et al. (2019) Scientific Reports, 9: 4929).

FIG. 2 shows the presence of Amy1 genes using gene-specific primers and melt curve analysis. A series of graphical representations of fluorescence (y-axis) and temperature (° C.; x-axis) for samples amplified using primers specific for Amy1-A1 (A), Amy1-B5 (B1), Amy1-B2 (B23) and Amy1-D1 (D) from (A) wild-type Chara; (B) A null mutant, showing loss of the A gene peak; (C) B null mutant, showing loss of the B1 and B23 gene peaks; (D) D null mutant, showing loss of the D gene peak.

FIG. 3 shows deletions in chromosomes 6A, 6B and 6D as detected by SNP analysis. (A) A nulls (21B_710) and partial A12null (21B_674); (B) B nulls (20B_241), B1 (22A_28) and partial nulls (20B_602); and (C) D null (19B_34) null. Arrows represent the location of the SNP detected by the 90K marker. Numbers indicate the base pair on the respective chromosome. The highlighted section between the two arrow heads indicates the chromosome segment identified as deleted using the SNP Chip method described elsewhere herein.

FIG. 4 shows the grain weight (g; y-axis) of single null and double null Amy1 mutants (x-axis) as calculated by measuring the weight of 1000 grains from each line.

FIG. 5 shows the grain composition analysis of single null and double null Amy1 mutants. A series of graphical representations of (A) total starch (% in wholemeal; y-axis); (B) soluble sugar content (mg/g in wholemeal; y-axis); (C) mixed link ≈-glucan content (g/kg in wholemeal; y-axis); and (D) protein content (%; y-axis) in single null and double null Amy1 mutants (x-axis).

FIG. 6 shows the starch properties and α-amylase activity of single null and double null Amy1 mutants. A series of graphical representations of (A) starch viscosity (rapid visco unit, RVU; y-axis); (B) proportion of small B starch granules (%; y-axis); (C) peak gelatinization temperature (° C.; y-axis); and (D) total α-amylase activity (Ceralpha unit, CU/g flour; y-axis) in single null and double null Amy1 mutants (x-axis).

FIG. 7 shows the falling number (seconds; y-axis) of single null, double null and triple null Amy1 mutants as compared to the flour standard and the Chara control line (x-axis).

FIG. 8 shows the total α-amylase activity (CU/g flour) single null, double null and triple null Amy1 mutants as compared to the flour standard (i.e., MACE) and the Chara control line (x-axis). Low levels of α-amylase activity are marked with *.

FIG. 9 shows the detection of specific TaAMY1 and TaAMY2 peptides (%; y-axis) from single null, double null and triple null Amy1 mutants (x-axis) using multiple reaction monitoring (MRM) analysis. Percentage of TaAMY1 (black bars) and TaAMY2 (lined bars) peptide relative to sprouting control is shown.

FIG. 10 shows the detection of LMA (%; y-axis) in single null, double null and triple null Amy1 mutants (x-axis) using ELISA. Percentage of LMA positive grains in the embryo (black bars) and endosperm (lined bars) is shown.

FIG. 11 shows that lines comprising the Bn mutant are resistant to LMA following exposure to a severe environmental trigger. A graphical representation of LMA positive grains (ELISA OD; y-axis) in mutants A12nBn and Bn as compared to An, Dn and Chara (x-axis). Each group of samples is separated by vertical black lines and the horizontal line represents the relative positivity threshold.

FIG. 12 shows the grain and grain composition analysis of the AMY1KO field trial. A series of graphical representations of (A) biomass (g; y-axis); (B) head number (y-axis); (C) grain yield (g; y-axis); and (D) harvest index (y-axis) in single, double and triple null Amy1 mutants (x-axis).

### BRIEF DESCRIPTION OF THE SEQUENCES

Nucleic acid sequences are referred to by a sequence identifier number (SEQ ID NO), with reference to the accompanying sequence listing.

SEQ ID NO: 1 shows the genomic DNA (gDNA) sequence of Amy1-6A-1.

SEQ ID NO: 2 shows the complementary DNA (cDNA) sequence of Amy1-6A-1.

SEQ ID NO: 3 shows the coding sequence (CDS) of Amy1-6A-1.

SEQ ID NO: 4 shows the amino acid sequence of the AMY1 protein encoded by SEQ ID NO: 1, 2 or 3.

SEQ ID NO: 5 shows the gDNA sequence of Amy1-6A-2.

SEQ ID NO: 6 shows the cDNA sequence of Amy1-6A-2.

SEQ ID NO: 7 shows the CDS of Amy1-6-A2.

SEQ ID NO: 8 shows the protein sequence of AMY1 protein encoded by SEQ ID NO: 5, 6 or 7.

SEQ ID NO: 9 shows the gDNA sequence of Amy1-6A-3.

SEQ ID NO: 10 shows the cDNA sequence of Amy1-6A-3.

SEQ ID NO: 11 shows the CDS sequence of Amy1-6A-3.

SEQ ID NO: 12 shows the amino acid sequence of the AMY1 protein encoded by SEQ ID NO: 9, 10 or 11.

SEQ ID NO: 13 shows the gDNA sequence of Amy1-6B-1.

SEQ ID NO: 14 shows the cDNA sequence of Amy1-6B-1.

SEQ ID NO: 15 shows the CDS sequence of Amy1-6B-1.

SEQ ID NO: 16 shows the amino acid sequence of the AMY1 protein encoded by SEQ ID NO: 13, 14 or 15.

SEQ ID NO: 17 shows the gDNA sequence of Amy1-6B-2.

SEQ ID NO: 18 shows the cDNA sequence of Amy1-6B-2.

SEQ ID NO: 19 shows the CDS sequence of Amy1-6B-2.

SEQ ID NO: 20 shows the amino acid sequence of the AMY1 protein encoded by SEQ ID NO: 17, 18 or 19.

SEQ ID NO: 21 shows the gDNA sequence of Amy1-6B-3.

SEQ ID NO: 22 shows the cDNA sequence of Amy1-6B-3.

SEQ ID NO: 23 shows the CDS sequence of Amy1-6B-3.

SEQ ID NO: 24 shows the amino acid sequence of the AMY1 protein encoded by SEQ ID NO: 21, 22 or 23.

SEQ ID NO: 25 shows the gDNA sequence of Amy1-6B-4.

SEQ ID NO: 26 shows the cDNA sequence of Amy1-6B-4.

SEQ ID NO: 27 shows the CDS sequence of Amy1-6B-4.

SEQ ID NO: 28 shows the amino acid sequence of the AMY1 protein encoded by SEQ ID NO: 25, 26 or 27.

SEQ ID NO: 29 shows the gDNA sequence of Amy1-6B-5.

SEQ ID NO: 30 shows the cDNA sequence of Amy1-6B-5.

SEQ ID NO: 31 shows the CDS sequence of Amy1-6B-5.

SEQ ID NO: 32 shows the amino acid sequence of the AMY1 protein encoded by SEQ ID NO: 29, 30 or 31.

SEQ ID NO: 33 shows the gDNA sequence of Amy1-6D-1.

SEQ ID NO: 34 shows the cDNA sequence of Amy1-6D-1.

SEQ ID NO: 35 shows the CDS sequence of Amy1-6D-1.

SEQ ID NO: 36 shows the amino acid sequence of the AMY1 protein encoded by SEQ ID NO: 33, 34 or 35.

SEQ ID NO: 37 shows the gDNA sequence of Amy1-6D-2.

SEQ ID NO: 38 shows the cDNA sequence of Amy1-6D-2.

SEQ ID NO: 39 shows the CDS sequence of Amy1-6D-2.

SEQ ID NO: 40 shows the amino acid sequence of the AMY1 protein encoded by SEQ ID NO: 37, 38 or 39.

SEQ ID NO: 41 shows the gDNA sequence of Amy1-6D-3.

SEQ ID NO: 42 shows the cDNA sequence of Amy1-6D-3.

SEQ ID NO: 43 shows the CDS sequence of Amy1-6D-3.

SEQ ID NO: 44 shows the amino acid sequence of the AMY1 protein encoded by SEQ ID NO: 41, 42 or 43.

SEQ ID NO: 45 shows the nucleic acid sequence of the forward primer TaAmy1_A1_F1.

SEQ ID NO: 46 shows the nucleic acid sequence of the reverse primer TaAmy1_genABD_R1.

SEQ ID NO: 47 shows the nucleic acid sequence of the forward primer Amy1_B1I1_F1.

SEQ ID NO: 48 shows the nucleic acid sequence of the forward primer Amy1_Bmix50I1_F1.

SEQ ID NO: 49 shows the nucleic acid sequence of the forward primer Amy1_D1I1_F1.

SEQ ID NO: 50 shows the nucleic acid sequence of the reverse primer Amy1_ABDI1_R.

SEQ ID NO: 51 shows the nucleic acid sequence of Amy1-6D from Triticum aestivum cultivar Chara (GenBank Accession No. KY368734.1).

SEQ ID NO: 52 shows the nucleic acid sequence of Amy1-6D from Triticum aestivum cultivar Chara (GenBank Accession No. KY368735.1).

SEQ ID NO: 53 shows the nucleic acid sequence of Amy1-6D from Triticum aestivum cultivar Chara (GenBank Accession No. KY368735.1).

SEQ ID NO: 54 shows the nucleic acid sequence of Amy1-6B from Triticum aestivum cultivar Chinese Spring (GenBank Accession No. KY368732.1).

SEQ ID NO: 55 shows the nucleic acid sequence of Amy1-6B from Triticum aestivum cultivar Chara (GenBank Accession No. KY368731.1).

SEQ ID NO: 56 shows the nucleic acid sequence of Amy1-6A from Triticum aestivum cultivar Chara (GenBank Accession No. KY368730.1).

SEQ ID NO: 57 shows the nucleic acid sequence of Amy1-6A from Triticum aestivum cultivar Chara (GenBank Accession No. KY368729.1).

SEQ ID NO: 58 shows the nucleic acid sequence of Amy1-6A from Triticum aestivum (GenBank Accession No. KY368728.1).

SEQ ID NO: 59 shows the amino acid sequence of a reduced and alkylated AMY1-A1 peptide.

SEQ ID NO: 60 shows the amino acid sequence of a reduced and alkylated AMY1-A1 peptide.

SEQ ID NO: 61 shows the amino acid sequence of a reduced and alkylated AMY1-A1 peptide.

SEQ ID NO: 62 shows the amino acid sequence of a reduced and alkylated AMY1-A2 peptide.

SEQ ID NO: 63 shows the amino acid sequence of a reduced and alkylated AMY1-A2 peptide.

SEQ ID NO: 64 shows the amino acid sequence of a reduced and alkylated AMY1-A2 peptide.

SEQ ID NO: 65 shows the amino acid sequence of a reduced and alkylated AMY1-A2 peptide.

SEQ ID NO: 66 shows the amino acid sequence of a reduced and alkylated AMY1-A2 peptide.

SEQ ID NO: 67 shows the amino acid sequence of a reduced and alkylated AMY1-A2 peptide.

SEQ ID NO: 68 shows the amino acid sequence of a reduced and alkylated AMY1-A2 peptide.

SEQ ID NO: 69 shows the amino acid sequence of a reduced and alkylated AMY1-A2 peptide.

SEQ ID NO: 70 shows the amino acid sequence of a reduced and alkylated AMY1-A2 peptide.

SEQ ID NO: 71 shows the amino acid sequence of a reduced and alkylated AMY1-A2 peptide.

SEQ ID NO: 72 shows the amino acid sequence of a reduced and alkylated AMY1-A2 peptide.

SEQ ID NO: 73 shows the amino acid sequence of a reduced and alkylated AMY1-A2 peptide.

SEQ ID NO: 74 shows the amino acid sequence of a reduced and alkylated AMY1-A2 peptide.

SEQ ID NO: 75 shows the amino acid sequence of a reduced and alkylated AMY1-A2 peptide.

SEQ ID NO: 76 shows the amino acid sequence of a reduced and alkylated AMY1-A2 peptide.

SEQ ID NO: 77 shows the amino acid sequence of a reduced and alkylated AMY1-A2 peptide.

SEQ ID NO: 78 shows the amino acid sequence of a reduced and alkylated AMY1-A3 peptide.

SEQ ID NO: 79 shows the amino acid sequence of a reduced and alkylated AMY1-B2 peptide.

SEQ ID NO: 80 shows the amino acid sequence of a reduced and alkylated AMY1-B2 peptide.

SEQ ID NO: 81 shows the amino acid sequence of a reduced and alkylated AMY1-B5 peptide.

SEQ ID NO: 82 shows the amino acid sequence of a reduced and alkylated AMY1-D1 peptide.

SEQ ID NO: 83 shows the amino acid sequence of a reduced and alkylated AMY1-D1 peptide.

SEQ ID NO: 84 shows the amino acid sequence of a reduced and alkylated AMY1-D1 peptide.

SEQ ID NO: 85 shows the amino acid sequence of a reduced and alkylated AMY1-D1 peptide.

SEQ ID NO: 86 shows the amino acid sequence of a reduced and alkylated AMY1-D3 peptide.

SEQ ID NO: 87 shows the amino acid sequence of a reduced and alkylated AMY1-D3 peptide.

SEQ ID NO: 88 shows the amino acid sequence of a reduced and alkylated AMY1-D3 peptide.

SEQ ID NO: 89 shows the amino acid sequence of a reduced and alkylated AMY1-D3 peptide.

SEQ ID NO: 90 shows the amino acid sequence of a reduced and alkylated AMY1-D3 peptide.

SEQ ID NO: 91 shows the amino acid sequence of a reduced and alkylated AMY1-D3 peptide.

SEQ ID NO: 92 shows the amino acid sequence of a reduced and alkylated AMY1-D3 peptide.

SEQ ID NO: 93 shows the amino acid sequence of a reduced and alkylated AMY1-D3 peptide.

SEQ ID NO: 94 shows the amino acid sequence of a reduced and alkylated AMY2-A2 peptide.

SEQ ID NO: 95 shows the amino acid sequence of a reduced and alkylated AMY2-A2 peptide.

SEQ ID NO: % shows the amino acid sequence of a reduced and alkylated AMY2-B1 peptide.

SEQ ID NO: 97 shows the amino acid sequence of a reduced and alkylated AMY2-B1 peptide.

SEQ ID NO: 98 shows the amino acid sequence of a reduced and alkylated AMY2-B1 peptide.

SEQ ID NO: 99 shows the amino acid sequence of a reduced and alkylated AMY2-B1 peptide.

SEQ ID NO: 100 shows the amino acid sequence of a reduced and alkylated AMY2-B1 peptide.

SEQ ID NO: 101 shows the amino acid sequence of a reduced and alkylated AMY2-B1 peptide.

SEQ ID NO: 102 shows the amino acid sequence of a reduced and alkylated AMY2-B1 peptide.

SEQ ID NO: 103 shows the amino acid sequence of a reduced and alkylated AMY2-B1 peptide.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a treatment” includes a single treatment, as well as two or more treatments; and so forth.

In the context of this specification, the term “about” in relation to a numerical value or range is intended to cover numbers falling within 10% of the specified numerical value or range.

Each embodiment in this specification is to be applied mutatis mutandis to every other embodiment, unless expressly stated otherwise.

Genes and other genetic material (e.g., mRNA, constructs, etc.) are represented in italics and their proteinaceous expression products are represented in non-italicized form. Thus, for example, AMY1 is an expression product of Amy1.

Nucleotide and amino acid sequences are referred to by a sequence identifier number (i.e., SEQ ID NO:). The SEQ ID NOs: correspond numerically to the sequence identifiers <400>1 (SEQ ID NO: 1), <400>2 (SEQ ID NO: 2), etc. A sequence listing is provided after the claims. A list describing the SEQ ID NOs in the sequence listing is provided above under the section “Brief Description of the Sequences”.

All sequence identifiers (e.g., GenBank ID, EMBL-Bank ID, DNA Data Bank of Japan (DDBJ) ID, etc.) provided herein were current at the filing date.

Throughout this specification, unless the context requires otherwise, the word “comprise”, or variations such as “comprises” or “comprising”, will be understood to mean the inclusion of a stated element or integer or group of elements or integers but not the exclusion of any other element or integer or group of elements or integers.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

The present invention is based in part on the surprising observations made in the experiments described herein that a wheat plant comprising a genetic modification leading to reduced expression of at least one Amy1 gene on chromosome 6B is sufficient to reduce the level or activity, or both, of alpha-amylase 1 (AMY1) polypeptides in the wheat grain, thereby reducing the susceptibility of the resulting grain to the deleterious effects of late maturity alpha-amylase (LMA). In certain embodiments described herein, grain derived from the wheat plants are further characterized by one or more, or all, of the beneficial features including a Falling Number of at least 200 seconds, reduced total alpha-amylase level, and reduced expression of AMY1 polypeptides in the grain. Each reduction is relative to the expression, level or activity of the component in wild-type wheat grain, or for wild-type wheat grain grown under the same conditions.

Wheat Plants with Reduced Susceptibility to Late Maturity Alpha-Amylase (LMA)

Accordingly, in an aspect, the present disclosure provides a wheat plant comprising a genetic modification leading to reduced expression of at least one Amy1 gene on chromosome 6B relative to a wild-type wheat plant, wherein grain from the plant is characterized by a reduced level or activity, or both, of alpha-amylase 1 (AMY1) polypeptides relative to a wild-type wheat plant.

The terms “alpha-amylase”, “α-amylase”, “AMY”, “%-1,4-glucan-4-glucanohydrolases” and “EC 3.2.1.1” may be used interchangeably herein to refer to a hydrolase that cleaves α-1,4 glycosidic linkages in starch, glycogen and other related oligosaccharides. The two major isoforms of alpha-amylase found in wheat, AMY1 and AMY2, have been isolated based on biochemical isoelectric point (pI). As used herein, the term “AMY1” refers to the high-pI α-amylase isoform, and the term “AMY2” refers to the low-pI α-amylase isoform.

“AMY1” polypeptides are encoded by multiple Amy1 genes located on chromosome 6 and when expressed produce AMY1 in the aleurone layer of the mature grain. Expression of AMY1 is initiated at the latest phase of grain development during desiccation and close to maturity.

“AMY2” polypeptides are encoded by multiple Amy2 genes located on chromosome 7 and when expressed produce AMY2 in the pericarp of the developing grain. Accumulation of AMY2 is thought to increase from anthesis and peaks with the initiation of the grain filling stage.

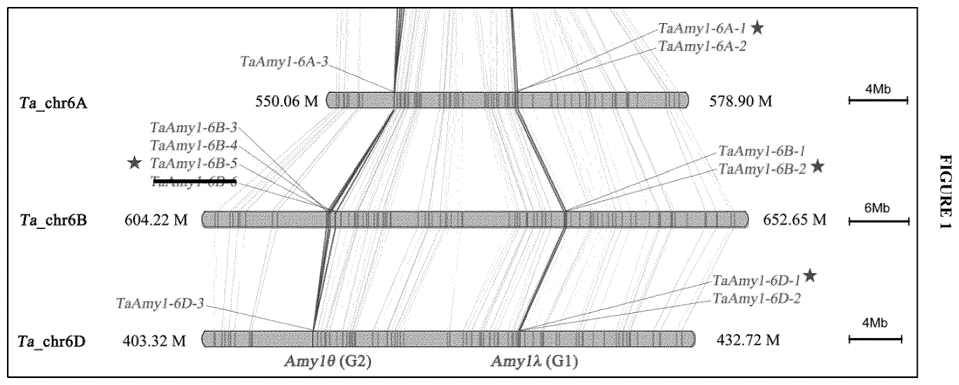

The Amy1 loci is a complex set of 12 genes divided in two separate clusters on each of the three wheat chromosome 6. The A and D genome contain three genes, while the B genome contains five active genes and one pseudogene. The Amy1 genes on chromosome 6B are split across two distinct clusters, the first containing four genes (i.e., three active genes and a pseudo gene) and the second containing 2 genes (i.e., two active genes). The Amy1 loci is schematically represented in FIG. 1.

Persons skilled in the art will appreciate that the Amy1 genes may be variously described by reference to their chromosomal location and relevant genome, for example, Amy1 genes on chromosome 6A may be referred to as Amy1-6A gene; Amy1 genes on chromosome 6B may be referred to as Amy1-6B genes; and Amy1 genes on chromosome 6D may be referred to as Amy1-6D genes.

In an embodiment, the at least one Amy1 gene on chromosome 6B is selected from Amy1-6B-1, Amy1-6B-2, Amy1-6B-3, Amy1-6B-4 and Amy1-6B-5.

The term “gene” as used herein is to be taken in the broadest context to include the deoxyribonucleotide sequences comprising the protein coding region of a structural gene and sequences located adjacent to the coding region on both the 5′ and 3′ ends for a distance of at least about 2 kilobases (kb) on either end. The sequences which are located 5′ of the coding region and which are present on the mRNA are referred to as “5′ non-translated sequences”. The sequences which are located 3′ or downstream of the coding region and which are present on the mRNA are referred to as 3′ non-translated sequences. The term “gene” encompasses by cDNA are genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecules encoding all or part of the proteins of the invention described herein and a complementary nucleotide sequence to any one of the above. The term “gene” as used herein also includes homeoforms.

An allele is a variant of a gene at a single genetic locus. Hexaploid wheat such as Triticum aestivum L. has six sets of chromosomes with a genome organization of AABBDD. Each chromosome has one copy of each gene (one allele). If both alleles of a chromosome pair are the same, the organism is homozygous with respect to that gene, if the alleles are different, the organism is heterozygous with respect to that gene. The interaction between alleles at a locus is generally described as dominant or recessive.

As used herein, the term “wheat” means a plant, plant part, grain or product derived therefrom of the species Triticum aestivum L. or Triticum turgidum ssp. durum or Triticale.

Triticum aestivum L., also known as “breadwheat”, is a hexaploid wheat with a genome organization of AABBDD, comprised of 42 chromosomes. The “A”, “B” and “D” subgenomes of Triticum aestivum L. are often referred to as “genomes”. Triticum turgidum ssp. durum, often referred to as “durum wheat” is a tetraploid wheat which has a genome organization of AABB, having 28 chromosomes. The term “wheat” as used herein is intended to encompass plants that are produced by conventional techniques using Triticum aestivum L. as a parent in a sexual cross with the non-Triticum species Secale cereale (rye), which hybrid progeny are referred to herein as “Triticale”. Preferably, the wheat plant is suitable for commercial production of grain, such as commercial varieties of breadwheat, having suitable agronomic characteristics which are known to those skilled in the art.

The term “plant” as used herein refers to a whole plant, parts thereof obtained from or derived from, such as, e.g., leaves, stems, roots, flowers, single cells (e.g., pollen), seeds, grain, plant cells and the like.

The term “genetic modification” as used herein refers to any modification to an Amy1 gene that leads to a reduction in the expression of an Amy1 gene on chromosome 6B. The wheat plants of the present disclosure can be produced and identified after mutagenesis. In some embodiments, the wheat plant is non-transgenic. In another embodiment, the wheat plant is transgenic.

In an embodiment, the wheat plant has reduced expression of at least two, at least three, or at least four Amy1 genes on chromosome 6B. In another embodiment, the wheat has reduced expression of all five Amy1 genes on chromosome 6B.

The term “reduced expression” as used herein means a level of expression that is lower than observed in wheat plants in the absence of the genetic modification. It is to be understood that the term “reduced” as used herein, does not necessarily imply that the expression of an Amy1 gene on chromosome 6B has been eliminated or is reduced to an undetectable level. In some embodiments, the level of expression of the at least one Amy1 gene on chromosome 6B may be reduced by at least about 40% (e.g., at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

In an embodiment, the expression of the at least one Amy1 gene on chromosome 6B is reduced to an undetectable level. Persons skilled in the art will appreciate that a reduction is expression to an undetectable level is intended to encompass embodiments whereby the expression of at least one Amy1 gene is effectively abolished.

In an embodiment, the genetic modification produces the reduced expression of at least one Amy1 gene on chromosome 6B via gene silencing.

The term “gene silencing” as used herein refers to the reduction of expression of a target nucleic acid in a wheat cell, preferably an endosperm cell during seed development, which can be achieved by the introduction of a silencing RNA. In some embodiments, a gene silencing chimeric gene is introduced into a wheat cell, which encodes a RNA molecule which reduces the expression of one or more endogenous genes, at least one of the Amy1-B6 genes. Such reduction may be the result of reduction of transcription, including by methylation of promoter regions via chromatin re-modelling, or post-transcriptional modification of the RNA molecules, including via RNA degradation, or both. Gene silencing should not necessarily be interpreted as an abolishing the expression of the target nucleic acid or gene. It is sufficient that the level expression of the target nucleic acid in the presence of the silencing RNA is lower than that observed in the absence thereof. Accordingly, in some embodiments, the level of expression of the targeted gene may be reduced by at least about 40% (e.g., at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

Methods for gene silencing would be known to persons skilled in the art, illustrative examples of which include the stable introduction and transcription of a gene silencing chimeric gene, sense-suppression techniques, anti-sense techniques and RNA interference (RNAi).

Antisense techniques may be used to reduce gene expression in wheat cells. The term “antisense RNA” as used herein means an RNA molecule that is complementary to at least a portion of a specific mRNA molecule and capable of reducing expression of the gene encoding the mRNA, preferably an Amy1-6B gene. Such reduction typically occurs in a sequence-dependent manner and is thought to occur by interfering with a post-transcriptional event such as mRNA transport from nucleus to cytoplasm, mRNA stability or inhibition of translation. The use of antisense methods is well known in the art, see, e.g., Hartmann and Endres, 1999, Manual of Antisense Methodology, Kluwer.

As used herein, the phrase “artificially-introduced dsRNA molecule” refers to the introduction of double-stranded RNA (dsRNA) molecule, which preferably is synthesized in the wheat cell by transcription from a chimeric gene encoding such dsRNA molecule. RNA interference (RNAi) is particularly useful for specifically reducing the expression of a gene or inhibiting the production of a particular protein, and is known to be effective in wheat (see, e.g., Regina et al., 2006, Proceedings of the National Academy of Science U.S.A., 103: 3546-3551). This technology relies on the presence of dsRNA molecules that contain a sequence that is essentially identical to the mRNA of the gene of interest or part thereof, and its complement, thereby forming a dsRNA. Conveniently, the dsRNA can be produced from a single promoter in the host cell, where the sense and anti-sense sequences are transcribed to produce a hairpin RNA in which the sense and anti-sense sequences hybridize to form the dsRNA region with a related (to a Amy1-6B gene) or unrelated sequence forming a loop structure, so the hairpin RNA comprises a stem-loop structure. The design and production of suitable dsRNA molecules for the present invention is well within the capacity of a person skilled in the art, as described by, e.g., Waterhouse et al. (1998, Proceedings of the National Academy of Science U.S.A., 95: 13959-13964; Smith et al. (2000, Nature, 407: 319-320); WO 1999/32619; WO 1999/53050; WO 1999/49029; and WO 2001/34815.

The DNA encoding the dsRNA typically comprises both sense and antisense sequences arranged as an inverted repeat. In a preferred embodiment, the sense and antisense sequences are separated by a spacer region which may (or may not) comprise an intron which, when transcribed into RNA, is spliced out. This arrangement has been shown to result in a higher efficiency of gene silencing (Smith et al., 2000, supra). The double-stranded region may comprise one or two RNA molecules, transcribed from either one DNA region or two. The dsRNA may be classified as long hpRNA, having long, sense and antisense regions which can be largely complementary, but need not be entirely complementary (typically larger than about 200 bp, e.g., between 200 and 1000 bp). A hpRNA can also be rather small with the double-stranded portion ranging in size from about 30 to about 42 bp, but not much longer than 94 bp, see, e.g., WO 2004/073390. The presence of the double stranded RNA region is thought to trigger a response from an endogenous plant system that destroys both the double stranded RNA and also the homologous RNA transcript from the target plant gene(s), efficiently reducing or eliminating the activity of the target gene.

The length of the sense and antisense sequences that hybridize should each be at least 19 contiguous nucleotides (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides, and so on), preferably at least 21 contiguous nucleotides, 30 contiguous nucleotides, 50 contiguous nucleotides, and more preferably at least 100, 200, 500 or 1000 contiguous nucleotides. The full-length sequence corresponding to the entire gene transcript may be used. The lengths are most preferably 100-2000 nucleotides. The degree of identity of the sense and antisense sequences to the targeted transcript should be at least 85% (e.g., at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively identical, i.e., 100%), preferably at least 90% and more preferably 95-100%. The longer the sequence, the less stringent the requirement for the overall sequence identity. The RNA molecule may of course comprise unrelated sequences which may function to stabilize the molecule. The promoter used to express the dsRNA-forming construct may be any type of promoter that is expressed in the cells which express the target gene, preferably a promoter which is preferentially expressed in the endosperm of the developing wheat grain relative to non-grain tissues of the wheat plant.

As used herein, “silencing RNAs” are RNA molecules that have 21 to 24 contiguous nucleotides that are complementary to a region of the mRNA transcribed from the target gene, preferably one or more Amy1 genes on chromosome 6B. The sequence of the 21 to 24 nucleotides is preferably fully complementary to a sequence of 21 to 24 contiguous nucleotides of the mRNA i.e., identical to the complement of the 21 to 24 nucleotides of the region of the mRNA. However, miRNA sequences which have up to five mismatches in region of the mRNA may also be used (Palatnik et al., 2003, Nature, 425: 257-263), and base-pairing may involve one or two G-U base-pairs. When not all of the 21 to 24 nucleotides of the silencing RNA are able to base-pair with the mRNA, it is preferred that there are only one or two mismatches between the 21 to 24 nucleotides of the silencing RNA and the region of the mRNA. With respect to the miRNAs, it is preferred that any mismatches, up to the maximum of five, are found towards the 3′ end of the miRNA. In a preferred embodiment, there are not more than one or two mismatches between the sequences of the silencing RNA and its target mRNA.

Silencing RNAs derived from longer RNA molecules, also referred to herein as “precursor RNAs”, are the initial products produced by transcription from the chimeric DNAs in the wheat cells and have partially double-stranded character formed by intra-molecular base-pairing between complementary regions. The precursor RNAs are processed by a specialized class of RNAses, commonly called “Dicer(s)”, into the silencing RNAs, typically of 21 to 24 nucleotides long. Silencing RNAs as used herein include short interfering RNAs (siRNAs) and microRNAs (miRNAs), which differ in their biosynthesis. siRNAs derive from fully or partially double-stranded RNAs having at least 21 contiguous base-pairs, including possible G-U base-pairs, without mismatches or non-base-paired nucleotides bulging out from the double-stranded region. These double-stranded RNAs are formed from either a single, self-complementary transcript which forms by folding back on itself and forming a stem-loop structure, referred to herein as a “hairpin RNA”, or from two separate RNAs which are at least partly complementary and that hybridize to form a double-stranded RNA region. miRNAs are produced by processing of longer, single-stranded transcripts that include complementary regions that are not fully complementary and so form an imperfectly base-paired structure, so having mismatched or non-base-paired nucleotides within the partly double-stranded structure. The base-paired structure may also include G-U base-pairs. Processing of the precursor RNAs to form miRNAs leads to the preferential accumulation of one or more distinct, small RNAs each having a specific sequence, the miRNA(s). They are derived from one strand of the precursor RNA, typically the “antisense” strand of the precursor RNA, whereas processing of the long complementary precursor RNA to form siRNAs produces a population of siRNAs which are not uniform in sequence but correspond to many portions and from both strands of the precursor.

miRNA precursor RNAs, also termed herein as “artificial miRNA precursors”, are typically derived from naturally occurring miRNA precursors by altering the nucleotide sequence of the miRNA portion of the naturally-occurring precursor so that it is complementary, preferably fully complementary, to the 21 to 24 nucleotide region of the target mRNA, and altering the nucleotide sequence of the complementary region of the miRNA precursor that base-pairs to the miRNA sequence to maintain base-pairing. The remainder of the miRNA precursor RNA may be unaltered and so have the same sequence as the naturally occurring miRNA precursor, or it may also be altered in sequence by nucleotide substitutions, nucleotide insertions, or preferably deletions, or any combination thereof. The remainder of the miRNA precursor RNA is thought to be involved in recognition of the structure by the Dicer enzyme called Dicer-like 1 (DCL1), and therefore it is preferred that few if any changes are made to the remainder of the structure. For example, base-paired nucleotides may be substituted for other base-paired nucleotides without major change to the overall structure. The use of artificial miRNAs have been demonstrated in plants, see, e.g., Alvarez et al. (2006, Plant Cell, 18: 1134-1151), Parizotto et al. (2004, Genes and Development, 18: 2237-2242), and Schwab et al. (2006, Plant Cell, 18: 1121-1133).

In an embodiment, the genetic modification is a mutation selected from a deletion mutation, an insertion mutation, a substitution mutation, a splice-site mutation, a premature translation termination mutation, a nonsense mutation and a frameshift mutation.

Wheat plants having a mutation in a single Amy1 gene on chromosome 6B can be produced and identified after mutagenesis. This may provide a wheat plant which is non-transgenic, which is desirable in some markets, or which is free of exogenous nucleic acid molecules. Generally, a progenitor plant cell, tissue, seed, or plant may be subjected to mutagenesis to produce single or multiple mutations, such as nucleotide substitutions, deletions, additions and/or codon modification.

Mutagenesis can be achieved by chemical or radiation means, e.g., ethyl methanesulfonate (EMS) or sodium azide (Zwar and Chandler, 1995, Planta, 197: 39-48) treatment of seed, or gamma irradiation, each of which would be known to persons skilled in the art. Chemical mutagenesis tends to favor nucleotide substitutions rather than deletions. Heavy ion beam (HIB) irradiation is known as an effective technique for mutation breeding to produce new plant cultivars, as described e.g., by Hayashi et al. (2007, Cyclotrons and their Applications, Eighteenth International Conference; 237-239) and Kazama et al. (2008 Plant Biotechnology, 25: 113-117). Ion beam irradiation has two physical factors, the dose (gy) and LET (linear energy transfer, keV/um) for biological effects that determine the amount of DNA damage and the size of DNA deletion, and these can be adjusted according to the desired extent of mutagenesis. HIB generates a collection of mutants, many of them comprising deletions that may be screened for mutations in specific Amy1 genes. The extent of the deletions generated by HIB may be determined by microsatellite mapping, e.g., using microsatellite markers previously mapped to the chromosomal locations of the Amy1-6B genes. Mutant plants where either all of most of the specific chromosome microsatellite markers are retained are inferred to be relatively small deletion mutants (e.g., 1-30 base pairs; 1-500 base pairs). Where either none or a few of the specific chromosome microsatellite markers are retained, relatively large deletion mutants are inferred (e.g., >500 base pairs). As described elsewhere herein, deletions generated using HIB that include at least a part of, or the whole of, and Amy1-6B gene can extend from 2 million bases up to 370 million bases (e.g., Example 3). Mutants which are identified may be backcrossed with non-mutated wheat plants as recurrent parents in order to remove and therefore reduce the effect of unlinked mutations in the mutagenized genome.

It is also contemplated herein that mutations can be introduced using biologically-based mutagenesis techniques. Suitable biologically-based mutagenesis techniques would be known to persons skilled in the art, illustrative examples of which include the use of biological agents for producing site-specific mutants, e.g., CRISPR, endonucleases, ZFNs, meganucleases, etc.

As used herein the term “biological agents” means any agent useful in producing site-specific mutants and includes enzymes that induce double stranded breaks in DNA that stimulate endogenous repair mechanisms. These include endonucleases, zinc finger nucleases, TAL effector proteins, transposases, site-specific recombinases and are CRISPR endonucleases. Zinc finger nucleases (ZFNs), e.g., facilitate site-specific cleavage within a selected gene within a genome allowing endogenous or other end-joining repair mechanisms to introduce deletions or insertions to repair the gap. Zinc finger nuclease technology is described in Le Provost et al. (2009, Trends in Biotechnology, 28(3): 134-141), Durai et al. (2005, Nucleic Acids Research, 33: 5978-5990) and Liu et al. (2010, Biotechnology and Bioengineering, 106: 97-105).

Isolation of mutants may be achieved by screening mutagenized plants or seed. For example, a mutagenized population of wheat may be screened directly for the Amy1-6B genotype or indirectly by screening for a phenotype that results from mutations in the Amy1-6B genes. Screening directly for the genotype preferably includes assaying for the presence of mutations in the Amy1-6B genes, which may be observed in PCR assays by the absence of specific Amy1-6B markers as expected when some of the genes are deleted, heteroduplex based assays as in TILLING (see, e.g., Slade and Knauf (2005, Transgenic Research, 14: 109-115), and Henikoff et al. (2004, Plant Physiology, 135: 630-636), or by deep sequencing. Screening is preferably based on nucleotide sequencing which is often based on pools of candidate mutants. Screening for the phenotype may comprise screening for a loss or reduction in amount of one or more AMY polypeptides or peptides by ELISA, mass spectroscopy or affinity chromatography, altered total alpha-amylase activity in the grain by the method described by Whan et al. (2014, Journal of Experimental Botany, 65(18): 5443-5457), or by measuring the Falling Number of the grain by the AACC Method 56-81.04.

Mutagenesis may be conducted in the desired elite genetic background. Alternatively, identified mutations may be introduced into desirable genetic backgrounds by crossing the mutant with a plant of the desired genetic background and, optionally, by performing a suitable number of backcrosses to cross out the originally undesired parent background.

The terms “induced mutation” or “introduced mutation” as used herein refer to an artificially induced genetic modification which may be the result of chemical, radiation or biological agent treatment of a progenitor cell, seed or plant, or may be the result of transposon or T-DNA insertion. Mutations may also be “introduced” into desirable genetic backgrounds by crossing the mutant with a plant of the desirable genetic background. Nucleotide insertional derivatives include 5′ and 3′ terminal fusions as well as intra-sequence insertions of single or multiple nucleotides. Insertional nucleotides sequence variants are those in which one or more nucleotides are introduced into a site in the nucleotide sequence, either at a predetermined site as is possible with ZFNs, TAL effectors, CRISPR or homologous recombination methods, or by random insertion with suitable screening of the resulting product. Deletion variants are characterized by the removal of one or more nucleotides from the sequence. Substitutional nucleotide variants are those in which at least one nucleotide in the sequence has been removed and a different nucleotide inserted in its place. In some embodiments, the number of nucleotides affected by substitutions in a mutant gene relative to the wild-type gene is a maximum of ten nucleotides, more preferably a maximum of 9, 8, 7, 6, 5, 4, 3, or 2, or most preferably only one nucleotide.

The term “mutation” as used herein does not include silent nucleotide substitutions which do not affect the gene activity. The term “polymorphism” refers to any change in the nucleotide sequence including such silent nucleotide substitutions. Screening methods may first involve screening for polymorphisms and secondly for mutations within a group of polymorphic variants. As described elsewhere herein, mutations include deletions of all or part of a gene, insertions such as an insertion into an exon of a gene, nucleotide substitutions, splice-site mutations, premature translation termination mutations, frameshift mutations, nonsense mutations and any combination thereof.

Any molecular biological technique known in the art that is capable of detecting one or more Amy1 genes on chromosome 6B can be used in the methods described herein. Such methods would be known to persons skilled in the art, illustrative examples of which include nucleic acid amplification, nucleic acid sequences, nucleic acid hybridization with suitably labelled probes, single-strand confirmation analysis (SSCA), denaturing gradient gel electrophoresis (DGGE), heteroduplex analysis (HET), chemical cleavage analysis (CCM), catalytic nucleic acid cleavage, or high resolution melt analysis, as described in Example 2.

In an embodiment, the mutation is a deletion mutation, which deletes all or a part of at least one Amy1 gene on chromosome 6B.

The deletion may be extensive enough to include one or more exons or introns, both exons and introns, an intron-exon boundary, a part of the promoter, the translational start site, the entire gene or even chromosomal region encompassing two or more, or all, of the Amy1 genes on chromosome 6B. Deletions may also extend far enough to include at least part of, or the whole of, an Amy1-6B gene and one or more adjacent genes on the B genome.

In an embodiment, the deletion mutation is a deletion of about 1-30 base pairs.

In an embodiment, the deletion mutation is a deletion of about 1-500 base pairs.

In another embodiment, the deletion mutation is a deletion of >500 base pairs.

Insertions or deletions within the exons of the protein coding region of a gene which insert or delete a number of nucleotides which is not an exact multiple of three, thereby causing a change in the reading frame during translation, almost always abolish activity of the mutant gene comprising such insertion or deletion, such mutations are “null mutations”. Insertions or deletions within the exons of the protein coding region of a gene which insert or delete a number of nucleotides which is an exact multiple of three may or may not abolish activity of the gene comprising such insertion or deletion. In the case of a deletion of an exact multiple of three nucleotides, the deletion would be expected to inactivate the encoded polypeptide if the deleted nucleotides encode a highly conserved amino acid. Enzyme assays or phenotypic assays can be used to determine if insertion or deletion mutations are null mutations.

In an embodiment, the deletion mutation deletes all or part of a nucleic sequence of SEQ ID NO: 13, 17, 21, 25 and/or 29, or a nucleic acid sequence which is at least 91% identical to the sequence of SEQ ID NO: 13, 17, 21, 25 and/or 29. Accordingly, the sequence may be at least 91%, at least 92%, at least 93%, at least 94%, at least 95%, at least 96%, at least 97%, at least 98%, at least 99%, or 100% identical to the nucleic acid sequence of SEQ ID NO: 13, 17, 21, 25 and/or 29.

Methods for the determination of nucleic acid sequence identity would be known to persons skilled in the art, illustrative examples of which include computer programs that employ algorithms, such as BLAST (Atschul et al., 1990, Journal of Molecular Biology, 215(3): 403-410).

In an embodiment, the wheat plant is non-transgenic, i.e., do not contain an exogenous gene construct (i.e., transgene), which are preferred in some markets.

In another embodiment, the wheat plant is a transgenic wheat plant.

The term “transgenic wheat plant” as used herein refers to wheat plants and their progeny, which have been genetically modified using recombinant techniques. This would generally be to modulate the production of at least one polypeptide described herein in the desired plant or plant organ. Transgenic plant parts include all parts and cells of said plants which comprise the transgene such as, e.g., seeds, cultured tissues, callus and protoplasts. Transgenic plants contain genetic material that they did not contain prior to the transformation. The genetic material is typically stably integrated into the genome of the plant. The introduced genetic material may comprise sequences that naturally occur in the same species but in a rearranged order or in a different arrangement of elements, e.g., an antisense sequence or a sequence encoding a double-stranded RNA. Such plants are included herein in “transgenic plants”. In an embodiment, the transgenic plants are homozygous for each gene that has been introduced (e.g., transgene) so that their progeny do not segregate for the desired phenotype.

Methods for the transformation of wheat plants to introduce an exogenous nucleic acid molecule would be known to persons skilled in the art, illustrative examples of which include acceleration of genetic material coated onto micro particles directly into cells, transformation by Agrobacterium-mediated technology, and electroporation technology. In an embodiment, transgenic wheat plants are produced by Agrobacterium tumefaciens-mediated transformation procedures. Vectors comprising the desired nucleic acid construct may be introduced into regenerable wheat cells of tissue cultured plants or explants, or suitable plant cells, e.g., protoplasts.

As used herein, the term “corresponding non-transgenic plant” refers to a plant which is the same or similar in most characteristics, preferably isogenic or near-isogenic relative to the transgenic plant, but without the genetic modification. Preferably, the corresponding non-transgenic plant is of the same cultivar or variety as the progenitor of the transgenic plant of interest, or a sibling plant line which lacks the genetic modification, often termed a “segregant”, or a plant of the same cultivar or variety transformed with an “empty vector” construct, and may be a non-transgenic plant.

As used herein, the term “progeny” includes all offspring from a wheat plant, both the immediate and subsequent generations, and both plants and seed (grain). Progeny include the seeds and plants obtained after self-fertilization (“selfing”) and the grain and plants resulting from a cross between two parental plants, such as the F1 offspring (first generation), F2, F3, F4, etc., being the offspring from the second etc., generations after selfing of the F1 plants.

The reduced expression of at least one Amy1 gene on chromosome 6B results in a grain that is characterized by a reduced level or activity, or both of AMY1 polypeptides relative to wild-type wheat grain.

The terms “polypeptide” and “protein” are generally used interchangeably herein and are intended to include variants, mutants, modifications and/or derivatives of the AMY1 polypeptides as described herein. As used herein, “substantially purified polypeptide” refers to a polypeptide that has been separated from the lipids, nucleic acids, other peptides and other molecules with which it is naturally associated in its native state. Preferably, the substantially purified polypeptide is at least 60% free, more preferably at least 75% free, and more preferably at least 90% free from other components with which it is naturally associated. By “recombinant polypeptide” it is meant a polypeptide made using recombinant techniques, e.g., through the expression of a recombinant polynucleotide in a cell, preferably a wheat cell.

The term “peptide” as used herein refers to digestion products of alpha-amylase proteins. Exemplary AMY1 peptide sequences include SEQ ID NOs: 59-93, and exemplary AMY2 peptides sequences include SEQ ID NOs: 94-103.

The level of AMY1 polypeptides or peptides can be measured using any methods known in the art for the detection and/or quantification of protein expression. Such methods would be known to persons skilled in the art, illustrative examples of which include immunological detection methods, such as Western Blotting and enzyme-linked immunosorbent assay (ELISA), e.g., the LMA ELISA assay described by Verity et al. (1999, Cereal Chemistry, 76: 673-681), and analysis of AMY peptides by mass spectrometry, as described in Example 6.

Alternatively, or additionally, the activity of AMY1 polypeptides may be measured using any methods known in the art for the detection and/or quantification of enzyme activity. e.g., by milling the grain to wholemeal and assaying for AMY1-mediated alpha-amylase activity.

In an embodiment, the level or activity, or both of AMY1 polypeptides is reduced by reduced by at least about 40% relative to wild-type wheat grain. Accordingly, the level or activity or both of AMY1 polypeptides may be reduced by at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%.

In an embodiment, the grain of the wheat plant is further characterized by one or more, or all, of the features selected from:
a. a Falling Number of at least 200 seconds;
b. reduced total alpha-amylase activity relative to a control level;
c. reduced expression of AMY1 polypeptides or peptides in the grain relative to a control level;
d. has reduced susceptibility to deleterious effects of late-maturity alpha-amylase (LMA) relative to wild-type wheat grain.

Accordingly, in a preferred embodiment, the grain is characterized by a Falling Number of at least 200 seconds. “Falling Number” refers to the time in seconds required to stir and for the stirrer to fall a measured distance through a hot aqueous flow or meal undergoing liquefaction. Due to the ability of alpha-amylase to liquefy starch gel, the Falling Number provides a measure of grain quality based on enzyme activity. Current minimum Falling Number requirements for Australian wheat grades are provided in Table 4. Methods for the measurement of Falling Number are well known in the art, including the RVA SN test (AACC International Approved Method 56-81.04) as described in Example 6.

Thus, in an embodiment, the grain is characterized by a Falling Number of at least 200 seconds, preferably about 200 seconds, preferably about 201 seconds, preferably about 202 seconds, preferably about 203 seconds, preferably about 204 seconds, preferably about 205 seconds, preferably about 206 seconds, preferably about 207 seconds, preferably about 208 seconds, preferably about 209 seconds, preferably about 210 seconds, preferably about 211 seconds, preferably about 212 seconds, preferably about 213 seconds, preferably about 214 seconds, preferably about 215 seconds, preferably about 216 seconds, preferably about 217 seconds, preferably about 218 seconds, preferably about 219 seconds, preferably about 220 seconds, preferably about 221 seconds, preferably about 222 seconds, preferably about 223 seconds, preferably about 224 seconds, preferably about 225 seconds, preferably about 226 seconds, preferably about 227 seconds, preferably about 228 seconds, preferably about 229 seconds, preferably about 230 seconds, preferably about 231 seconds, preferably about 232 seconds, preferably about 233 seconds, preferably about 234 seconds, preferably about 235 seconds, preferably about 236 seconds, preferably about 237 seconds, preferably about 238 seconds, preferably about 239 seconds, preferably about 240 seconds, preferably about 241 seconds, preferably about 242 seconds, preferably about 243 seconds, preferably about 244 seconds, preferably about 245 seconds, preferably about 246 seconds, preferably about 247 seconds, preferably about 248 seconds, preferably about 249 seconds, preferably about 250 seconds, preferably about 251 seconds, preferably about 252 seconds, preferably about 253 seconds, preferably about 254 seconds, preferably about 255 seconds, preferably about 256 seconds, preferably about 257 seconds, preferably about 258 seconds, preferably about 259 seconds, preferably about 260 seconds, preferably about 261 seconds, preferably about 262 seconds, preferably about 263 seconds, preferably about 264 seconds, preferably about 265 seconds, preferably about 266 seconds, preferably about 267 seconds, preferably about 268 seconds, preferably about 269 seconds, preferably about 270 seconds, preferably about 271 seconds, preferably about 272 seconds, preferably about 273 seconds, preferably about 274 seconds, preferably about 275 seconds, preferably about 276 seconds, preferably about 277 seconds, preferably about 278 seconds, preferably about 279 seconds, preferably about 280 seconds, preferably about 281 seconds, preferably about 282 seconds, preferably about 283 seconds, preferably about 284 seconds, preferably about 285 seconds, preferably about 286 seconds, preferably about 287 seconds, preferably about 288 seconds, preferably about 289 seconds, preferably about 290 seconds, preferably about 291 seconds, preferably about 292 seconds, preferably about 293 seconds, preferably about 294 seconds, preferably about 295 seconds, preferably about 296 seconds, preferably about 297 seconds, preferably about 298 seconds, preferably about 299 seconds, preferably about 300 seconds, preferably about 301 seconds, preferably about 302 seconds, preferably about 303 seconds, preferably about 304 seconds, preferably about 305 seconds, preferably about 306 seconds, preferably about 307 seconds, preferably about 308 seconds, preferably about 309 seconds, preferably about 310 seconds, preferably about 311 seconds, preferably about 312 seconds, preferably about 313 seconds, preferably about 314 seconds, preferably about 315 seconds, preferably about 316 seconds, preferably about 317 seconds, preferably about 318 seconds, preferably about 319 seconds, preferably about 320 seconds, preferably about 321 seconds, preferably about 322 seconds, preferably about 323 seconds, preferably about 324 seconds, preferably about 325 seconds, preferably about 326 seconds, preferably about 327 seconds, preferably about 328 seconds, preferably about 329 seconds, preferably about 330 seconds, preferably about 331 seconds, preferably about 332 seconds, preferably about 333 seconds, preferably about 334 seconds, preferably about 335 seconds, preferably about 336 seconds, preferably about 337 seconds, preferably about 338 seconds, preferably about 339 seconds, preferably about 340 seconds, preferably about 341 seconds, preferably about 342 seconds, preferably about 343 seconds, preferably about 344 seconds, preferably about 345 seconds, preferably about 346 seconds, preferably about 347 seconds, preferably about 348 seconds, preferably about 349 seconds, or more preferably about 350 seconds.

In an embodiment, the grain is characterized by a Falling Number of at least 250 seconds, preferably at least 300 seconds or more preferably at least 350 seconds.

In another preferred embodiment, the in a preferred embodiment, the grain is characterized by reduced total alpha-amylase activity relative to a control level.

“Total alpha-amylase activity” refers to the level of alpha-amylase activity in wheat grain. By “total alpha-amylase” it is meant the activity of any and all alpha-amylases in a sample, with no discrimination between different isoforms of alpha-amylase (e.g., AMY1, AMY2, AMY3, etc.). Suitable methods for measuring total alpha-amylase would be known to persons skilled in the art, illustrative examples of which include the detection of p-nitrophenol from 4,6-O-ethylidene-α-4-nitrophenyl-maltoheptaoside by α-amylase in the presence of α-glucosidase, for example, using the Megazyme Ceralpha kit described in Examples 5 and 6.

In an embodiment, the total alpha-amylase activity is from about 1% to about 50% of the total alpha-amylase activity of grain relative to a control level. Thus, in an embodiment, the total alpha-amylase activity is from about 1% to about 50%, preferably about 1%, preferably about 2%, preferably about 3%, preferably about 4%, preferably about 5%, preferably about 6%, preferably about 7%, preferably about 8%, preferably about 9%, preferably about 10%, preferably about 11%, preferably about 12%, preferably about 13%, preferably about 14%, preferably about 15%, preferably about 16%, preferably about 17%, preferably about 18%, preferably about 19%, preferably about 20%, preferably about 21%, preferably about 22%, preferably about 23%, preferably about 24%, preferably about 25%, preferably about 26%, preferably about 27%, preferably about 28%, preferably about 29%, preferably about 30%, preferably about 31%, preferably about 32%, preferably about 33%, preferably about 34%, preferably about 35%, preferably about 36%, preferably about 37%, preferably about 38%, preferably about 39%, preferably about 40%, preferably about 41%, preferably about 42%, preferably about 43%, preferably about 44%, preferably about 45%, preferably about 46%, preferably about 47%, preferably about 48%, preferably about 49%, or more preferably about 50% of the total alpha-amylase activity of the control level.

In yet another preferred embodiment, the grain is characterized by reduced expression of AMY1 polypeptides or peptides in the grain relative to a control level.

The localization of AMY1 polypeptide expression may also be used to differentiate grains affected by LMA from other sprouting-related damage, e.g., pre-harvest sprouting (PHS). In particular, and as described elsewhere herein, LMA is characterized by the accumulation and persistent of AMY1 polypeptides in the aleurone throughout the harvest, and can be detected more broadly throughout embryonic and non-embryonic (i.e., endosperm) tissue in the grain. Accordingly, in an embodiment, the grain is characterized by reduced expression of AMY1 polypeptides in the embryo and endosperm of the grain relative to a control level.

In an embodiment, the expression of AMY1 polypeptides or peptides in the grain is reduced by at least 50% of the expression of AMY1 polypeptides or peptides relative to the control level. Thus, in an embodiment, the expression of AMY1 polypeptides or peptides is reduced by at least 50%, preferably at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100% relative to the control level.

The term “control level” as used herein refers to a level (i.e., expression level) or activity that may be used to compare the characteristics of the wheat plant or grain described herein. In certain embodiments, the control level will the level or activity in a wild-type wheat plant or grain. The term “wild-type” as used herein refers to a cell, tissue, plant or grain that has not been modified. Wild-type cells, tissue, or plants that are known in the art may be used as controls to compare the levels of expression of endogenous or exogenous nucleic acid molecules or polypeptides, or the extent and nature of trait modification in cells, tissue, or plants modified as described herein. As used herein, the term “wild-type wheat grain” means a wheat grain that has not been modified, e.g., non-mutagenized, non-transgenic. Specific wild-type wheat grains as used herein include, but are not limited to, Chara.

The skilled person will appreciate that where a comparison is made between the plants or the grain of the present disclosure and those which are wild-type, the comparison is performed with plants grown under essentially identical growing conditions, growth time, temperature, water and nutrient supply, etc., and for grain obtained from such plants.

In another embodiment, the control level is a known or predetermined level or activity that may be used to distinguish the wheat plants or grain of the present disclosure from other wheat plants or grains, e.g., the control level may be a pre-determined level of total alpha-amylase activity that is typical of wheat plants or grain that are affected by LMA.

Any one or more of the characteristics of Falling Number, level of total alpha-amylase activity and expression of AMY1 polypeptides or peptides in the grain may provide an indication that the grain is contaminated by LMA, when considered alone or in combination. Accordingly, in another preferred embodiment, the grain is characterized as having reduced susceptibility to LMA relative to wild-type wheat grain.

“Late-maturity alpha-amylase” or “LMA” is a grain quality defect resulting from abnormally high levels of alpha-amylase. As described elsewhere herein, LMA is characterized by elevated levels of AMY1 polypeptides, which are triggered by environmental stress during wheat grain development. AMY1 polypeptides accumulate and persist in the aleurone through the harvest, but are also present throughout the embryonic and non-embryonic (i.e., endosperm) tissue of the grain post-harvest. The detection of AMY1 polypeptides in both embryonic and non-embryonic tissue of grains can be used to distinguish grains affected by LMA from other sprouting damage, such as post-harvest sprouting.

The present inventors have shown that genetic modification leading to reduced expression of at least one Amy1 gene on chromosome 6B is sufficient to control the deleterious effects of AMY1 polypeptides, even in wheat plants that are grown in conditions suitable to trigger LMA. The identification of the Amy1-6B genes as the genes responsible for the upregulation of the expression of AMY1 polypeptides from the complex Amy1 loci has enabled the development of an alternative strategy to reduce susceptibility to LMA by controlling the deleterious effect caused by elevated AMY1 levels. The unexpected technical advantage of the approach described herein is the provision of wheat plants and grain with reduced susceptibility to LMA by controlling the down-stream release of AMY1 resulting from the complex mechanisms that triggers LMA. Accordingly, by “reduced susceptibility to LMA” it is meant that wheat plants, grain or cells comprising a genetic modification leading to reduced expression of at least one Amy1 gene on chromosome 6B have reduced susceptibility to the deleterious effects of LMA in wheat grain that is associated with the upregulation of alpha-amylase, and in particular AMY1 polypeptides. The wheat plants, grain and cells of the present disclosure may thus also be referred to as “LMA free”, which would be understood by persons skilled in the art to describe the effective control of the deleterious effects of LMA, i.e., reduced or eliminated susceptibility to LMA.

Reduced susceptibility to LMA may be determined using any one or more of the methods for the measurement of Falling Number, total alpha-amylase activity, the level of expression of AMY1 polypeptides, and the analysis of specific AMY peptides, as described elsewhere herein.

In an aspect, the present disclosure provides a method for determining susceptibility to LMA in a wheat plant, the method comprising:
a. obtaining a sample of grain from the wheat plant;
b. analyzing the sample for the presence of at least one AMY1 peptide selected from the amino acid sequences of SEQ ID NOs: 59-93, or an amino acid sequence which comprises not more than three modifications to an amino acid sequence of SEQ ID NOs: 59-93; and/or at least one AMY2 peptide selected from the amino acid sequences of SEQ ID NOs: 94-103, or an amino acid sequence which comprises not more than three modifications to an amino acid sequence of SEQ ID NOs: 94-103;
c. comparing the peptides analyzed in step (b) to those present in control wheat grain, and based on this comparison, determining if the wheat plant is susceptible to LMA.

In an embodiment, the control wheat grain is sprouted wheat grain.

In an embodiment, the sample of grain is milled or ground grain.

In another embodiment, the sample of grain is an extract of the grain.

In an embodiment, the AMY1 and/or AMY2 peptides are analyzed by targeted mass spectrometry.

“Targeted mass spectrometry” is a mass spectrometry technique that can selectively quantify compounds in mixtures. The targeted mass spectrometry data may be acquired through targeted acquisition methods, e.g., a Selective Reaction Monitoring (SRM) method and a Multiple Reaction Monitoring (MRM) method, which enable the detection and quantification (i.e., characterization) of alpha-amylase polypeptides in the sample. SRM is performed on triple quadrupole-like instruments, in which increased selectivity is obtained through collision-induced dissociation. It is a non-scanning mass spectrometry technique, where two mass analyzers are used as static mass filters, to monitor a particular fragment of a selected precursor. The specific pair of mass-over-charge (m/z) values associated to the precursor and fragment ions selected is referred to as a “transition”. The detector acts as a counting device for the ions matching the selected transition thereby returning an intensity distribution over time. MRM is when multiple SRM transitions are measured within the same experiment on the chromatographic time scale by rapidly switching between the different precursor/fragment pairs. Typically, the triple quadrupole instrument cycles through a series of transitions and records the signal of each transition as a function of the elution time. The method allows for additional selectivity by monitoring the chromatographic co-elution of multiple transitions for a given sample.

In an embodiment, the targeted mass spectrometry comprises a multiple reaction monitoring (MRM) method.

In an embodiment, the AMY1 peptides comprising the amino acid sequences of SEQ ID NOs: 59-93 and the AMY2 peptides comprising the amino acid sequences of SEQ ID NOs: 94-103 are analyzed in step (b).

In accordance with this embodiment, the wheat plant is determined to be susceptible to LMA when at least about 50% of the AMY1 peptides comprising the amino acid sequences of SEQ ID NOs: 59-93 are present in the sample relative to the control wheat grain (e.g., at least at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or all of the AMY1 peptides comprising the amino acid sequences of SEQ ID NOs: 59-93 are present, i.e., 100% relative to the control wheat grain); and less than about 50% of the AMY2 peptides comprising the amino acid sequence of SEQ ID NOs: 94-103 are present in the in the sample relative to the control wheat grain (e.g., less than about 50%, less than about 49%, less than about 48%, less than about 47%, less than about 46%, less than about 45%, less than about 44%, less than about 43%, less than about 42%, less than about 41%, less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, or effectively abolished to an undetectable level, i.e., 0% relative to the control wheat grain).

In an embodiment, from about 50% to about 100% of the AMY1 peptides comprising the amino acid sequences of SEQ ID NOs: 59-93 are present in the sample.

In an embodiment, from about 0% to about 50% of the AMY2 peptides comprising the amino acid sequences of SEQ ID NOs: 94-103 are present in the sample.

In an embodiment, the wheat plant is determined to have reduced susceptibility to LMA when:
a. less than about 30% of the AMY1 peptides comprising the amino acid sequences of SEQ ID NOs: 59-93 are present in the sample relative to the control wheat grain; and
b. less than about 30% of the AMY2 peptides comprising the amino acid sequence of SEQ ID NOs: 94-103 are present in the in the sample relative to the control wheat grain.

The wheat plants of the present disclosure preferably have additional traits/characteristics that are of agronomic benefit. Accordingly, in an embodiment, wheat grain derived from the wheat plant are further characterized by one or more of the features selected from:
a. a content of protein that is increased relative to wild-type wheat grain;
b. a proportion of total starch that is the same as, or reduced relative to wild-type wheat grain;
c. a content of soluble sugars that is the same as, or increased relative to wild-type wheat grain;
d. starch with reduced viscosity (peak viscosity, pasting temperature, etc.) relative to wild-type wheat grain; and
e. a content of beta-glucan that is the same as, or reduced, relative to wild-type wheat grain.

In another embodiment, the wheat plant is male and female fertile.

The wheat plants described herein may be crossed with plants containing a more desirable genetic background. The desired genetic background may include a suitable combination of genes providing commercial yield or other characteristic, such as agronomic performance, biotic or abiotic stress resistance. The genetic background may also include altered starch biosynthesis or other modification genes, e.g., genes from other wheat lines. In some embodiments, the genetic background may comprise one or more transgenes such as, e.g., a gene that confers fungal resistance. In another embodiment, the genetic background may comprise non-transgenic mutations that confer tolerance to a herbicide, such as mutations in the ALS gene conferring herbicide resistance.

In a another aspect of the present disclosure, there is provided a method of producing a plant with reduced susceptibility to LMA, the method comprising:
a. introducing a genetic modification to at least one Amy1 gene on chromosome 6B to a plant cell such that the cell has reduced level or activity, or both, of AMY1 polypeptides relative to unmodified cells;
b. regenerating a plant with the genetic modification from the cell of step (a), wherein the plant has reduced susceptibility to LMA relative to wild-type plants lacking the genetic modification.

In an embodiment, the method further comprises harvesting the seed from the plant.

In another embodiment, the method further comprises crossing the plant with the genetic modification with a second plant to produce one or more progeny plants.

It is also contemplated herein that the plant with the genetic modification may be selected for crossing to generate progeny plants and grain which have genetic modifications in multiple Amy1-6B genes. In certain embodiments, plants may be used in a series of crosses, backcrosses, intercrosses and progeny selections, e.g., F1 progeny plants from the crosses are selfed, and F2 seed obtained and analyzed for their Amy1-6B genotype. Such plants can also be crossed with plants comprising genetic modifications in other Amy1-6B genes to produce double or triple null gene mutants having combinations of genetic modifications in the Amy1 genes, as described in Example 4.

Accordingly, in certain embodiments, the second plant comprises a genetic modification in at least one Amy1 gene on chromosome 6B that differs from the first plant, e.g., the first plant may comprise mutations in Amy1-B3, Amy1-B4 and Amy1-B5 (e.g., B1n) and the second plant may comprise mutations in Amy1-B1 and Amy1-B2 (e.g., B23n).

In another embodiment, the second plant comprises a genetic modification in at least one Amy1 gene on chromosome 6A and/or 6D (e.g., Amy1-6A-1, Amy1-6A-2, Amy1-6A-3, Amy1-6D-1, Amy1-6D-2, and Amy1-6D-3)

### Grain

The present disclosure also provides wheat grain obtained from, or obtainable from, or which is a part of the wheat plant as described herein, or produced by the process as described herein.

In another aspect of the present disclosure, there is provided a wheat grain comprising a genetic modification leading to reduced expression of at least one Amy1 gene on chromosome 6B relative to a wild-type wheat grain, wherein the grain is characterized by a reduced level or activity, or both, of AMY1 polypeptides relative to a wild-type wheat grain.

As used herein, the term “grain” means grain as is typically harvested from mature wheat plants growing in the field, including grain used for food production or in food products, and germinated grain after it has been sowed before but before the emergence of seedlings. Grain also includes grain which has been processed for food production or which is an ingredient in a food product. The harvested wheat grain as described herein typically has a moisture content of about 10% to about 15% by weight. The parts of the grain include the testa (seed coat), the pericarp (fruit coat), the aleurone layer, the starchy endosperm and the embryo (germ) which is made up of the scutellum, the plumule (shoot) and radicle (primary root). The combined testa, pericarp and aleurone layer are commonly referred to as the “bran”, which can be removed from the grain by milling, which may also comprise the germ. The scutellum is the region that secretes some of the enzymes involved in germination and absorbs the soluble sugars from the breakdown of starch in the endosperm for growth of the seedling after germination. The aleurone which surrounds the starchy endosperm also secretes enzymes during germination.

In an embodiment, the wheat grain is further characterized by one or more, or all of the features selected from:
a. a Falling Number of at least 200 seconds;
b. reduced total alpha-amylase activity relative to a control level;
c. reduced expression of AMY1 polypeptides or peptides in the grain relative to a control level; and
d. has reduced susceptibility to the deleterious effects of LMA relative to a wild-type wheat grain.

The terms “seed” and “grain” as used herein have overlapping meanings. “Grain” includes seeds which have been harvested from a plant and generally refers to mature, harvested grain, but can also refer to grain after imbibition or germination, according to the context. “Seed” can refer to either mature grain, either before or after harvesting, or to immature seeds which are developing in planta. As described elsewhere herein, mature grain has a moisture content of less than about 10 to about 15% by weight.

In certain embodiments, it is preferred that the wheat grain is capable of growing into a wheat plant when the grain is sown into soil.

The present disclosure also provides a process for producing wheat grain, comprising:
a. growing a wheat plant as described herein, preferably in a field as part of a population of at least 1000 such plant or in an area of at least 1 hectare planted at a standard planting density; and
b. harvesting grain from the plant.

The present disclosure also provides a process for producing bins of wheat grain, comprising:
a. reaping above-ground parts of wheat plants as described herein;
b. threshing and/or winnowing the parts of the wheat plants to separate the grain from the remainder of the plant parts;
c. sifting and/or sorting the grain separated in step (b); and
d. loading the sifted and/or sorted grains into bins, thereby producing bins of grain.

In certain embodiments, the wheat grain has been processed so that it is no longer capable of germinating. This may be achieved by removal of the embryo from the seed, for example, by milling, or by heat treatment, or other processing of the grain. The grain may be kibbled, cracked, par-boiled, rolled, pearled, milled or ground grain.

The present disclosure also provides a method of producing wheat flour, wholemeal, starch, starch granules or bran, the method comprising obtaining the grain of the present disclosure and processing the grain to produce the flour, wholemeal, starch, starch granules or bran. Such processing methods are well known in the art. The step of obtaining the grain may comprise, e.g., harvesting grain from a wheat plant as described herein, or purchasing the grain.

The present disclosure also provides wheat flour, wholemeal, starch, starch granules or bran produced by the method as described herein, or comprising the nucleic acid molecule or the polypeptide as described herein.

The present disclosure also provides products produced from the plants or grain as described herein, such as a food product, which may be a food ingredient. Examples of food products include flour, starch, leavened or unleavened breads, pasta, noodles, breakfast cereals, snack foods, cakes, malt, pastries and foods containing flour-based sauces. The food product may be a bagel, a biscuit, a bread, a bun, a croissant, a dumpling, an English muffin, a muffin, a pita bread, a quick bread, a refrigerated/frozen dough product, dough, baked beans, a burrito, chili, a taco, a tamale, a tortilla, a pot pie, a ready to eat cereal, a ready to eat meal, stuffing, a microwavable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwavable snack product, a nutritional bar, a pancake, a par-baked bakery product, a pretzel, a pudding, a granola-based product, a snack chip, a snack food, a snack mix, a waffle, a pizza crust. In a preferred embodiment, the food product is leavened or unleavened bread, pasta, noodle, breakfast cereal, snack food, cake, pastry or a flour-based sauce.

In an embodiment, the food product as described herein may be prepared by mixing the grain, or flour, wholemeal or bran from the grain, with at least one other food ingredient. Accordingly, in an embodiment, there is provided a food product comprising the wheat grain, the wheat cell, the nucleic acid molecule, the polypeptide, or an ingredient which is the wheat flour, wholemeal, starch, starch granules or bran as described herein.

Wheat flour, wholemeal, starch, starch granules, bran and food products (e.g., food ingredients) described herein have beneficially improved quality relative to wheat flour, wholemeal, starch, starch granules, bran and food products (e.g., food ingredients) contaminated by LMA. For example, it is known in the art that excess levels of α-amylase in wheat flour used to produce bread disrupts the fermentation process and leads to sticky dough that is difficult to handle and produces bread with a sticky crumb, dark-colored crust and loaves with large holes that are difficult to slice and may jam slicing machines (see, e.g., Mares et al., 2004, Enzyme Activities in Wrigley (ed), Encyclopedia of Grain Science, Elsevier, pp 357-365).

### Cells

The present disclosure also provides a wheat cell derived from the wheat plant or wheat grain as described herein.

In an embodiment, the wheat cell is a regenerable wheat cell, which is capable of growing into a plant. Regenerable wheat cells include cells of mature embryos, meristematic tissue such as the mesophyll cells of the leaf base, or preferably from the scutella of immature embryos, obtained 12-20 days post-anthesis, or callus derived from any of these.

### Polynucleotides

In another aspect of the disclosure, there is provided a nucleic acid molecule which encodes an AMY1 polypeptide, wherein the nucleic acid molecule differs from the nucleic acid of SEQ ID NO: 13, 17, 21, 25 and/or 29, or a nucleic acid sequence which is at least about 91% identical to the nucleic acid sequence of SEQ ID NO: 13, 17, 21, 25 and/or 29, by a mutation selected from a deletion mutation, an insertion mutation, a splice-site mutation, a premature translation termination mutation, a nonsense mutation and a frameshift mutation.

The terms “polynucleotide”, “nucleic acid” and “nucleic acid molecule” mean a single- or double-stranded polymer of deoxyribonucleotide, ribonucleotide bases or known analogs of natural nucleotides, or mixtures thereof, and can include molecules comprising coding and non-coding sequences of a gene, sense and anti-sense sequences and complements, exons, introns, genomic DNA, cDNA, pre-mRNA, mRNA, rRNA, siRNA, miRNA, tRNA, ribozymes, recombinant polynucleotides, isolated and purified naturally-occurring sequences, synthetic RNA and DNA sequences, nucleic acid probes, primers and fragments.

In an embodiment, the nucleic acid molecule comprises a deletion mutation, which deletes all or a part of the sequence set forth as SEQ ID NO: 13, 17, 21, 25 and/or 29, or a nucleic acid sequence which is at least about 91% identical to the nucleic acid sequence of SEQ ID NO: 13, 17, 21, 25 and/or 29.

In an embodiment, the nucleic acid molecule comprises a deletion which is about 1-30 base pairs.

In an embodiment, the nucleic acid molecule comprises a deletion of about 1-500 base pairs.

In another embodiment, the nucleic acid molecule comprises a deletion of >500 base pairs.

In accordance with certain embodiments of the present disclosure, the nucleic acid molecule is in a cell, preferably in a wheat cell, or in a food product.

Transformation of a nucleic acid molecule into a cell can be accomplished by any method by which a nucleic acid molecule can be inserted into the cell. Transformation techniques include, but are not limited to, transfection, electroporation, microinjections, lipofection, adsorption, and protoplast fusion.

The nucleic acid molecule of the present disclosure may be operably linked to a promoter capable of driving expression of the nucleic acid molecule in a plant cell.

“Operably linking” a promoter or enhancer element to a nucleic acid molecule means placing the nucleic acid molecule (e.g., a protein encoding nucleic acid molecule or other transcript) under the regulatory control of a promoter, which then controls the transcription of that polynucleotide. In the construction of heterologous promoter/structural gene combinations, it is generally preferred to position a promoter or variant thereof at a distance from the transcription start site of the nucleic acid molecule, which is approximately the same as the distance between that promoter and the gene it controls in its natural setting, i.e., the gene from which the promoter is derived. As is known in the art, some variation in this distance can be accommodated without loss of function.

In certain embodiments, the nucleic acid molecule of the present disclosure may be included in a nucleic acid construct.

Nucleic acid constructs typically comprise one or more regulatory elements, such as promoters, enhancers, as well as transcription termination or polyadenylation sequences. Such elements are well known in the art. The transcription initiation region comprising the regulatory element(s) may provide for regulated or constitutive expression in the plant. The regulatory elements may be selected from, e.g., seed-specific promoters, or promoters not specific for seed cells (e.g., ubiquitin promoter or CaMV35S or enhanced 35S promoters). Seed-specific promoters would be known to persons skilled in the art, illustrative examples of which include, the wheat low molecular weight glutenin promoter (Colot et al., 1987, The EMBO Journal, 6: 3559-3564) and the hordein promoter (Brandt et al., 1985, Carlsberg Research Communications, 50: 333-345). The promoter may be modulated by factors such as temperature, light or stress. Ordinarily, the regulatory elements will be provided 5′ of the nucleic acid molecule to be expressed. The construct may also contain other elements that enhance transcription, such as the nos 3′ or the ocs 3′ polyadenylation regions or transcription terminators.

Typically, the nucleic acid construct comprises a selectable marker. Selectable markers aid in the identification and screening of plants or cells that have been transformed with an exogenous nucleic acid molecule. The selectable marker may provide antibiotic or herbicide resistance to the wheat cells, or allow the utilization of substrates, e.g., mannose.

Preferably, the nucleic acid construct is stably incorporated into the genome of a plant. Accordingly, the nucleic acid construct comprises additional elements, which allow the molecule to be incorporated into the genome, or the construct is placed in an appropriate vector, which can be incorporated into a chromosome of a plant cell.

In an embodiment, the nucleic acid construct is incorporated in a recombinant vector, which includes at least one nucleic acid molecule described herein, inserted into any vector capable of delivering the nucleic acid molecule into a host cell. Such a vector contains heterologous nucleic acid sequences, that is, nucleic acid sequences that are not naturally found adjacent to nucleic acid molecules of the present invention, which are preferably derived from a species other than the species from which the nucleic acid molecule(s) are derived. The vector can be either RNA or DNA, either prokaryotic or eukaryotic, and is typically is a virus or a plasmid.

The present disclosure also provides a method of genotyping a wheat plant or grain, the method comprising:
a. obtaining a sample comprising nucleic acid extracted from a wheat plant or grain; and
b. detecting in the sample a nucleic acid molecule as described herein.

The present disclosure also provides a method of selecting a wheat plant from a population of wheat plants, wherein said population of plants comprises progeny plants obtained from a cross between two plants of which at least one plant was a wheat plant as described herein, the method comprising:
a. genotyping each one or more progeny plants in said population of wheat plants using the method described herein; and
b. based on the results of the genotyping in step (a), selecting a progeny plant which comprises the nucleic acid molecule described herein.

In an embodiment, the progeny is genotyped for the presence or absence of the nucleic acid molecule described herein.

The present disclosure also provides a method for reducing susceptibility to LMA into a wheat plant, the method comprising:
a. crossing a first parent wheat plant with a second parent wheat plant, wherein the second plant is a wheat plant as described herein; and
b. backcrossing a progeny plant of the cross of step (a) with a plant of the same genotype as the first parent plant to produce a plant with a majority of genotype of the first parent but comprising a nucleic acid molecule as described herein.

In an embodiment, the progeny is genotyped for the presence or absence of the nucleic acid molecule described herein.

Accordingly, certain embodiments, the second parent wheat plant comprises a genetic modification in at least one Amy1 gene on chromosome 6B that differs from the first parent wheat plant, e.g., the first parent wheat plant may comprise mutations in Amy1-B3, Amy1-B4 and Amy1-B5 (e.g., B1n) and the second parent wheat plant may comprise mutations in Amy1-B1 and Amy1-B2 (e.g., B23n).

In another embodiment, the first parent wheat plant comprises a genetic modification in at least one Amy1 gene on chromosome 6A and/or 6D (e.g., Amy1-6A-1, Amy1-6A-2, Amy1-6A-3, Amy1-6D-1, Amy1-6D-2, and Amy1-6D-3).

### Polypeptides

In another aspect of the disclosure, there is provided an AMY1 polypeptide, wherein the polypeptide differs from the amino acid sequence of SEQ ID NO: 16, 20, 24, 28 and/or 32, or an amino acid sequence which is at least 99% identical to the amino acid sequence of SEQ ID NO: 16, 20, 24, 28 and/or 32, by at least one amino acid substitution, insertion or deletion in the active site, catalytic site, calcium binding site and/or carbohydrate binding domain, wherein the AMY1 polypeptide has an impaired structure and/or reduced activity relative to an AMY1 polypeptide having the amino acid sequence of SEQ ID NO: 16, 20, 24, 28 and/or 32.

As described herein, the AMY1 polypeptide differs from the amino acid sequence of SEQ ID NO: 16, 20, 24, 28 and/or 32, or an amino acid sequence which is at least 99% identical to the amino acid sequence of SEQ ID NO: 16, 20, 24, 28 and/or 32, by at least one amino acid substitution, insertion or deletion in the active site, catalytic site, calcium binding site and/or carbohydrate binding domain, wherein the AMY1 polypeptide has an impaired structure and/or reduced activity relative to an AMY1 polypeptide having the amino acid sequence of SEQ ID NO: 16, 20, 24, 28 and/or 32. Accordingly, the amino acid sequence may be at least 99% or 100% identical to the amino acid sequence of SEQ ID NO: 16, 20, 24, 28 and/or 32.

Methods for the determination of amino acid sequence identity would be known to persons skilled in the art, illustrative examples of which include computer programs that employ algorithms, such as protein BLAST (Altschul et al., 1997, Nucleic Acids Research, 25: 3389-3402).

In an embodiment, the polypeptide differs from the amino acid sequence of SEQ ID NO: 32 by at least one amino acid substitution, insertion or deletion in:
a. the active site, wherein the at least one amino acid substitution, insertion or deletion is between amino acid 33 and 312 of SEQ ID NO: 32;
b. the catalytic site, wherein the at least one amino acid substitution, insertion or deletion is between amino acid 203 and 313 of SEQ ID NO: 32; and/or
c. the calcium binding site, wherein the at least one amino acid substitution, insertion or deletion is between amino acid 115 and 172 of SEQ ID NO: 32.

In an embodiment, the at least one amino acid substitution, insertion or deletion is selected from:
a at least one amino acid substitution, insertion or deletion of an amino acid corresponding to amino acid W33, Y75, H116, F167, R201, F204, K206, W230, T231 or H312 of SEQ ID NO: 32;
b. at least one amino acid substitution, insertion or deletion of an amino acid corresponding to amino acid D203, E228 or D313 of SEQ ID NO: 32; and/or
c. at least one amino acid substitution, insertion or deletion of an amino acid corresponding to amino acid N115 or D172 of SEQ ID NO: 32.

In an embodiment, the at least one amino acid substitution, insertion or deletion is between amino acid 294 and 418 of SEQ ID NO: 24.

In an embodiment, the at least one amino acid substitution, insertion or deletion corresponds to amino acid Y400 of SEQ ID NO: 24.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### Example 1—General Materials and Methods

### Plant Materials

### Glasshouse

Wheat plants, including the Chara controls (i.e., the heavy ion bombarded (HIB) population in Chara background), were grown a CSIRO Agriculture Black Mountain Innovation Park, Canberra, in a glasshouse with natural light and at temperatures of 18° C. (night) and 24° C. (day). Plants were grown in 15 cm pots and watered daily. Developing grain samples (dissected where appropriate) and other tissue samples were frozen on dry ice and stored at −80° C. until time of analysis.

### Glasshouse Growth Study

The growth study was performed using the method described by Ral et al. (2012, Plant Biotechnology Journal, 10(7): 871-82).

### Germination Study

Germination assays were performed as described by Jacobsen et al. (2013, Planta, 238: 121-138). Grains were harvested at physiological maturity and four weeks after physiological maturity on the mother plant. The protocol for harvest followed that of Gubler et al. (2008, Plant Physiology, 147: 886-896) with the following modifications: harvested grain was dried at 37° C. for 24 hours with low humidity and were then stored at −20° C. Grains were imbibed on 9 cm plastic Petri dishes containing 0.3% agarose (w/v) and MilliQ water. Plates were incubated at 20° C. in the dark. Coleorhiza emergence (CE) was counted every 24 hours for the duration of the experiment.

### Field Trial

Field trials on selected Amy1 single, double and triple knockout mutants alongside the control varieties Chara and Crusader, were grown in a replicated split plot trial at the CSIRO Boorowa Research Station (NSW, Australia). Nitrogen treatments meeting standard agronomic practice for the region of the trial were applied at sowing and again just prior to anthesis. Single null An, Bn and Dn had six replicated plots, while the rest were sown in triplicate. Harvest cuts were taken from two locations in each plot at maturity. All stalks in a 90 cm² frame were cut at ground level and processed for biomass, harvest index and thousand grain weight in the laboratory. Plots were machine harvested. Plot yield was standardized by dividing by plot area to calculate kg·m⁻². Field trial information is summarized in Table 1.

### Grain Weight

Grain was harvested from plants at maturity, which was taken as when the plants were completely yellowed. The heads were harvested and stored at 37° C. for at least two weeks to ensure complete drying, then stored at room temperature if further storage was required, and then threshed to provide mature grain.

Grain weight for each wheat line was determined from the total weight of 100 grains. The moisture content of grain was measured with a MPA FT-NIR spectrometer (BRUKER); this was typically about 9% on a weight basis for mature grain. Parameters that depend on the grain weight such as starch content, BG content, fructan content, etc. as described herein were calculated on a dry weight basis, assuming a 9% moisture content (w/w) if not measured by NIR Unless otherwise specified, 5 g of dried grain per line was milled using a Udy Cyclone mill (Fort Collins, CO, USA) with 0.5 mm mesh screen to generate wholemeal flour or using a Brabender Quadrumat Junior mill (Brabender® GmbH & Co. KG, Duisburg, Germany) to obtain white flour.

### Alpha-Amylase Assay

Total alpha-amylase (α-amylase) activity was assessed in 10 mg wholemeal samples and 206 ground developing grain samples using the CERALPHA kit (Megazyme International Ireland Ltd) and the method described by Whan et al. (2014, supra), as adapted for 96-well format and with appropriate dilutions. Data was expressed in Ceralpha Unit (CU) per gram of flour or μg of protein as determined by Bradford assay.

### Mutant Screening

DNA extracted from the HIB mutant population described by Li et al. (2011, in: Wheat Breeding Assembly, August 24-26, Citigate Hotel, Perth, Western Australia: 95) was used to screen for single null mutants in the α-amylase alleles. Screening for mutants was performed using single-channel real-time PCR and multiplexed melt curve analyses. The analyses includes a DNA concentration/quality check and produce unequivocal results for null mutants. Primers to detect the alleles were designed and are shown in Table 2.

### Identification of Deletions by 90K SNP Analysis

To analyze and identify the location of the deletions from the HIB population mutants a 90K single nucleotide polymorphism (SNP) array was performed using a custom 90K Wheat BeadChip for Illumina Infinium SNP genotyping in accordance with the method of Sichen et al. (2014, Plant Biotechnology Journal, 12(6): 787-796).

Raw intensity data for the 7 Amy1 HIB mutant lines (as well as data generated for the Chara controls and additional control group of 96 unrelated HIB mutants) was loaded into GenomeStudio and the NormTheta (x-axis coordinate) and NormR (y-axis coordinate) values from SNP cluster plots was exported. Shifts in cluster position were calculated between the euploid Chara and each of the mutant samples or between the average HIB position of the control group of 96 unrelated HIB mutants and each of the Amy1 mutant samples. The control HIBs were used to assist in the identification of putative deletions in the event that the Chara controls included biotypes (i.e., different versions of Chara).

SNP loci putatively tracking deleted segments were identified in each of the Amy1 HIB mutants. SNPs with a minimum cluster shift between the Chara controls and the additional control group of HIB mutants of 0.1 along the x-axis (i.e., NormTheta) or 0.5 along the y-axis (i.e., NormR), were considered to be putative markers for a deletion.

To clarify the position of the Amy1 genes in chromosomes 6A, 6B and 6D, sequences similar to the Amy1 gene sequence (KJ470678) were aligned in Gydle against the NRgene Chinese Spring genome assembly. Regions in the wheat NRgene scaffolds containing Amy1-like sequence were identified from the alignment. Each distinct region of the genome containing an Amy1-like sequence was numbered sequentially according to the sub-genome and position in the genome e.g., 6A1, 6A2, 6A3, 6B1, and so on. 90K probes containing hybridization sites within the NRgene wheat scaffolds were identified. In order to validate that deletions were located on the sub-genome of interest (i.e., 6A) rather than a homeologoue (i.e., 6B or 6D), 90K SNPs with only one hybridization site in the NRgene assembly were identified and single-site SNPs located near either end of the deletion were visually assessed in GenomeStudio. A “null” allele (i.e., where there is no fluorescence due to deletion of the hybridization site) were considered to be indicative of the deletion occurring on the sub-genome specified by the NRgene scaffold.

Deleted segments were identified in each HIB line. Markers were ordered by chromosome, then by their position in the NRgene assembly. Markers putatively flanking deleted segments were identified for each HIB line. Markers were visually assessed in GenomeStudio to assess polymorphism relative to Chara. To estimate the size of the deletion, information for the NRgene scaffolds (HiC Bin, PopSeq Chr, PopSeq cM) and 90K mapping information (SNP consensus map) were reported.

### Grain Composition Analysis

Grain compositional analysis was performed according to the methods described in Ral et al. (2012, supra) and Whan et al. (2014, supra).

### Starch Extraction

Unless stated otherwise, starch was extracted from grain samples by first grinding grain (10 g) to wholemeal using a Cyclone mill machine (Cyclote 1093, Tecator, Sweden). Starch was isolated from the wholemeal by a protease extraction method (Morrison et al., 1984, Journal of Cereal Science, 2(4): 257-271), and washed with water using 10 ml of water per gram of wholemeal, at room temperature, with removal of the tailings. The starch was then freeze-dried and weighed for analysis. Starch is also isolated on a small scale from developing wheat grain using the method of Regina et al. (2006, supra).

### Starch Content

The total starch content of grain was assayed by AACC method 76.13, using the total starch analysis kit (K-TSTA) supplied by Megazyme (Bray, Co Wicklow, Republic of Ireland) and calculated on a weight basis as a percentage of the mature, unmilled grain weight. Subtraction of the starch weight from the total grain weight to give a total non-starch content of the grain determined whether the reduction in total weight was due to a reduction in starch content.

### Soluble Sugars

Total soluble sugar was measured using anthrone method (Whan et al., 2014, supra) and compared to a standard curve established using anthrone solution with glucose gradient (1 mg·mL⁻¹ glucose stock solution).

### Beta-Glucan (β-Glucan)

β-glucan was measured using Mixed-linkage β-glucan assay kit (Megazyme International Ireland, Ltd.) which permits the analysis of small scale samples (20 mg) by its procedure with some modification. Three technical replicates were performed on each sample.

### Starch Gelatinization

The gelatinisation temperature profiles of starch samples were measured in a Pyris 1 differential scanning calorimeter (Perkin Elmer, Norwalk CT, USA). The viscosity of starch solutions was measured on a Rapid-Visco-Analyser (RVA, Newport Scientific Pty Ltd, Warriewood, Sydney), for example, using conditions as reported by Batey et al., (1997, Cereal Chemistry, 74(4): 497-501). The parameters measured included peak viscosity (the maximum hot paste viscosity), holding strength, final viscosity and pasting temperature. The swelling volume of flour or starch was determined according to the method of Konik-Rose et al., (2001, Starch, 53(1): 14-20). The uptake of water was measured by weighing the sample prior to and after mixing the flour or starch sample in water at defined temperatures and following collection of the gelatinized material.

### Protein Content

Total protein of the wholemeal flour was determined by Dumas combustion method using DuMaster Buchi D-480 (Buchi, Switzerland). 2 mg of wholemeal was used per assay and three technical replicated were performed. The factor of 5.8 was used to convert nitrogen amounts into crude protein content.

### Falling Number

Determination of Falling Number was performed according to the AACC Method 56-81.04.

### MRM/Targeted MS Method Development

### Protein Extraction and Digestion

Triplicate samples from wholemeal flour from wheat lines and negative controls were prepared for α-amylase protein extraction. Milled wholemeal flour (50 mg) was dissolved in 500 ul of Ceralpha:α-amylase reagent (50 mM sodium malate, 50 mM sodium chloride, 2 mM calcium chloride, pH 5.4), vortexed and incubated with agitation, at ambient room temperature for 20 min. Samples were then heated to 40° C., shaken in a thermal mixer for half an hour and centrifuged at 17,000g for 10 min at room temperature. The supernatant was reduced by the addition of 1M DTT (dithiothreitol) at a 1:10 ratio to reach 100 mM DTT final concentration. This mixture was incubated for an hour at 40° C. and 14,000 rpm.

100 μl aliquots of the protein extracts were applied to a 10 kDa molecular weight cut-off filter (Millipore, Australia), washed twice with 200 μl of 8M urea, 100 mM Tris-HCL (pH8.5) and centrifuged at 17000 g for 10 min. For cysteine alkylation, 100 mM iodoacetamide in 8M urea, 100 mM Tris-HCL was added (100 μl) and incubated at room temperature in the dark for 30 min. The filters were centrifuged at 14 000 rpm for 10 min to remove excess iodoacetamide and washed twice using 100 μl of 8M urea, 100 mM Tris-HCl buffer.

### Global Proteomic Profiling and Protein Identification

Pooled samples (5 μL from each replicate, n=4) were analyzed as described previously (Colgrave et al., 2017, Journal of Agricultural and Food Chemistry, 65(44): 9715-9725) with chromatographic separation using a micro HPLC system (Shimadzu Scientific, Rydalmere, Australia) directly coupled to a 6600 TripleTOF MS (SCIEX, Redwood City, USA). ProteinPilot™ 5.0 software (SCIEX) with the Paragon Algorithm (Shilov et al., 2007, Molecular and Cellular Proteomics, 6(9): 1638-55) was used for protein identification. Tandem mass spectrometry data was searched against in silico proteolytic digests of Poaceae proteins of the Uniprot database (version 2017/02; 2,891,190 sequences) appended with the common repository of adventitious proteins (cRAP) database. The search parameters were defined as iodoacetamide modified for cysteine alkylation and trypsin as the digestion enzyme. ProteinPilot generates a score for each protein based on the confidence, wherein a confidence of 99% is assigned a score of 2.00 and a confidence of 95% is assigned a score of 1.30. The database search results from the combined barley cultivar analyses were manually curated to yield the protein identifications using a 1% global false discovery rate (FDR) determined by the in-built FDR tool within ProteinPilot software (Tang et al., 2008, Journal of Proteomics Research, 7(9): 3661-3667).

### Identification of α-Amylase Peptides and MRM/Targeted-MS

Mass spectrometry analysis was performed following the method described in Whan et al. (2014, supra). Peptide summaries generated by ProteinPilot were used to select peptides that yielded intense peaks and were fully tryptic, i.e., no variable or missed cleavages. MRM transitions were determined for each peptide where the precursor ion (Q1) m/z and the fragment ion (Q3) m/z values were determined from the data collected in the discovery experiments. In total, 54 peptides were selected for MRM experiments.

Reduced and alkylated digested peptides (20 L) were chromatographically separated on a Shimadzu Nexera UHPLC and analysed on a 6500 QTRAP mass spectrometer (SCIEX) as described previously (Colgrave et al., 2016, Analytical Chemistry, 88(18): 9127-9135) with minor modifications. Initially, four MRM transitions were analysed in MRM experiments. From these, three MRM transitions were selected based on transition intensity and lack of interferences. Relative quantitation was achieved using scheduled MRM scanning experiments using a 60 s detection window for each MRM transition with RT as determined in the preliminary MRM experiment and a 0.3 s cycle time. Peak area integration of each peptide was conducted using Skyline software v. 19.1 (MacLean et al., 2010, Bioinformatics, 26(7): 966-8). Three modifications carbamidomethyl (C), oxidation (M) and Gln->pyro-Glu (N-term Q) were allowed for each peptide and all transitions were required to co-elute at the same retention time (RT, min). The peak areas of the three MRM transitions monitored were summed and the data was converted to a percentage relative to the average peak area (of all extracts and fractions) for ease of data comparison. Graphs were generated in GraphPad Prism v6.

### Example 2—Identification of TaAMY1 Genes

The Amy1 locus is a complex set of 12 genes divided in two separated clusters on each of the three wheat chromosomes 6. The A and D genome contain 3 genes each while the B genome contains 6 genes divided in two clusters of 4 (3 active genes and a pseudo gene) and 2 respectively (FIG. 1).

To demonstrate the feasibility of combining null alleles of Amy1 as a viable strategy for the elimination of LMA, a dedicated screening method was established to assist mutation detection on large HIB population generated using the wheat variety Chara. Hexaploidy makes the wheat genome highly tolerant to mutations compared to diploid plants. As a result, two types of mutant populations with high mutation frequencies have been successfully generated in wheat: 1) TILLING populations where high-frequency point mutations are randomly induced by chemical agents, and 2) mutant populations where large deletions are created by bombardment from heavy ions as described in Li et al. (2011, supra).

DNA samples from wheat HIB null mutants for selected genes were available (20-400 ng/μL concentration range). Available DNA samples from wild-type Chara (˜50 ng/μL) were used for sequencing. Genome specific primer pair were designed to screen the HIB population as shown in Table 2.

Target genes were amplified for sequencing where necessary, using genome non-specific primers following the method described by Mieog et al. (2017, De Gruyter Open, 1: 35-49) with the following changes. To amplify the gene from all three genomes in the same reaction, PrimeSTAR Max DNA polymerase (TaKaRa, Clonetech Laboratories, Inc) was used according to the manufacturer's instructions. Products were TOPO cloned using a Zero Blunt Topo PCR cloning kit (Invitrogen) following the manufacturer's instructions. Up to 14 plasmids per target gene were sequenced (BigDye Terminator v3.1 Cycle Sequencing Kit, Thermo Fisher Scientific) with the M13 forward and reverse primers to ensure good reads for all three genomes were obtained. The primers were designed with reference to the full Amy genome, corresponding to GenBank Accession No. KY368734.1 (SEQ ID NO: 51); KY368735.1 (SEQ ID NO: 52), KY368735.1 (SEQ ID NO: 53), KY368732.1 (SEQ ID NO: 54), KY368731.1 (SEQ ID NO: 55), KY368730.1 (SEQ ID NO: 56), KY368729.1 (SEQ ID NO: 57), and KY368728.1 (SEQ ID NO: 58).

All melt runs were performed on a PikoReal real-time PCR machine (Thermo Fisher Scientific) and consisted of 5 μL Sensimix SYBR no-ROX (Bioline Pty Ltd), 0.3 μL primer premix, and 3.7 μL H₂O and 1 μL DNA template. Cycling conditions were standardized at 10 minutes at 95° C., then 40 cycles of 10 seconds at 95° C., 10 seconds at 60° C., 10 seconds at 72° C., followed by a melt curve analysis starting at 60° C. and increasing at 0.2° C. increments each second. Initial genome-specific test runs used forward and reverse primers premixes, both at 10 nM concentrations. Multiplexed primer premixes were optimized using trial-and-error with the highest primer pair concentration pre-set at 10 nM per primer.

Using sequence information from GenBank as well as in-house generated sequences, alignments containing representative sequences for all three genomes were made for all three target genes.

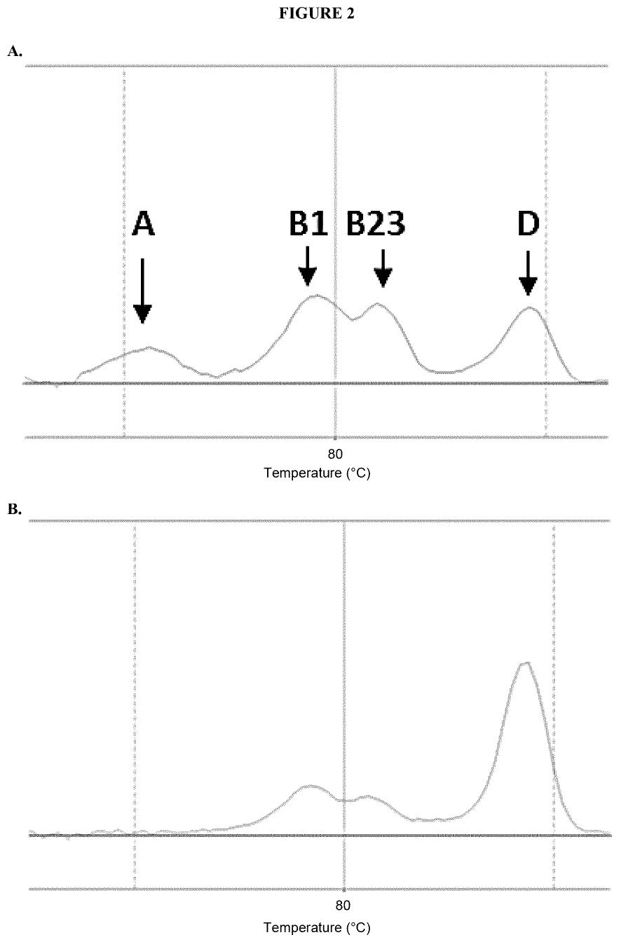

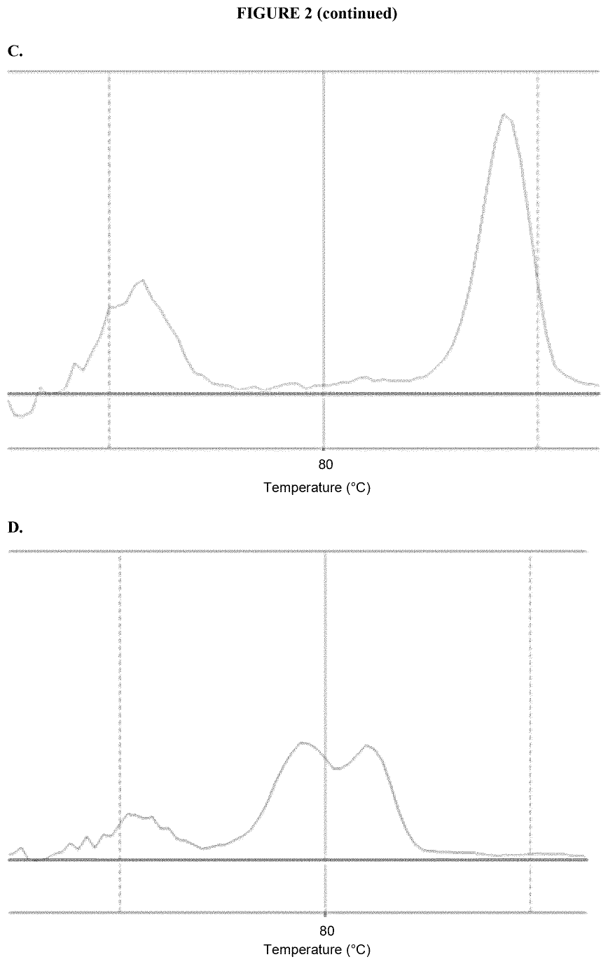

Analysis of presence of Amy1 genes using gene specific primers and melt curve analysis result is shown in FIG. 2. The primer pairs are specific for the following genes: Amy1-A1 (A), Amy1-B5 (B1), Amy1-B2 (B23) and Amy1-D1 (D). The presence of the individual 4 gene-specific peaks, representing the 4 genes detected, is shown for wild-type sample (WT). Data presented for the WT Chara plants indicate that all of the genes were detected using the 4 primer pairs (FIG. 2). The analysis was repeated for the lines obtained from the single and double null growth trial. Near identical results were obtained from the analysis of the single nulls from the single and double growth trial.

Lines from the HIB population containing deletions within the Amy1 loci were identified including in the A genome (8 lines), B (24) and D genome (8). More B genome mutants were identified in comparison to A and D genome mutants. While deletion removing simultaneously the two Amy1 cluster could be found for both A and B genome, only mutation removing Amy1 D1 and D2 could be detected No mutation impacting the full D genome locus (Amy1 D1, D2 and D3 simultaneously) was detected by the mutant population screening.

### Example 3—SNP Analysis to Identify Regions of Deletion in the HIB Population

Nine lines were selected for full genomic analysis of chromosome 6 using a 90K SNP chip analysis as described elsewhere herein. The lines selected were the two A nulls (21B_710 & 22A_183) and a partial A12 null (21B_674); B nulls (18A_509 & 20B_602), B1 (22A_28) & B23 (partial) nulls (20B_602 & 22A_7) and the D null (19B_34). Of the nine lines analyzed six of these lines were retained for further analysis as shown in Table 3.

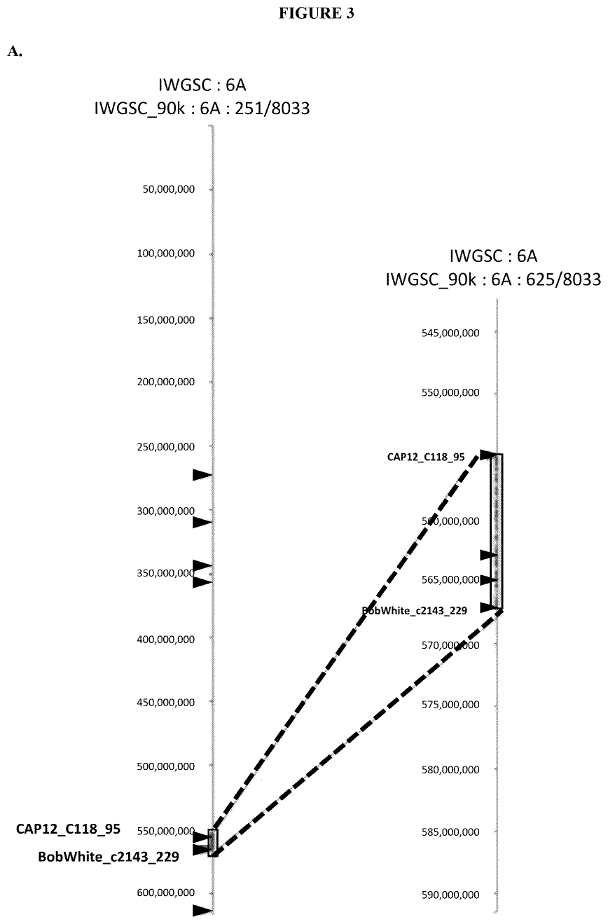

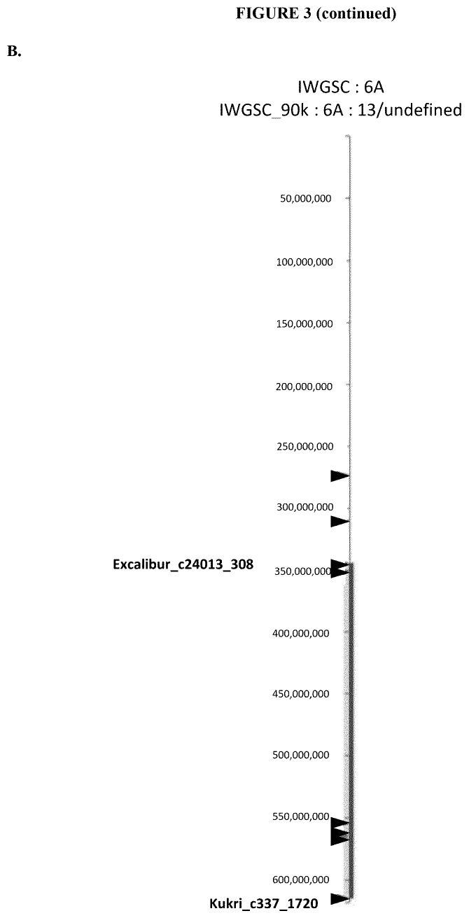

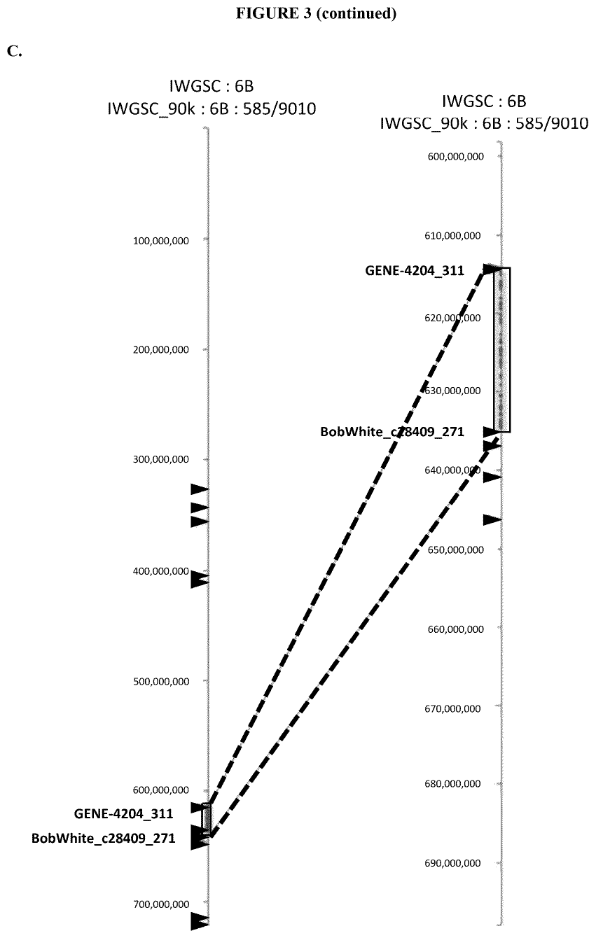

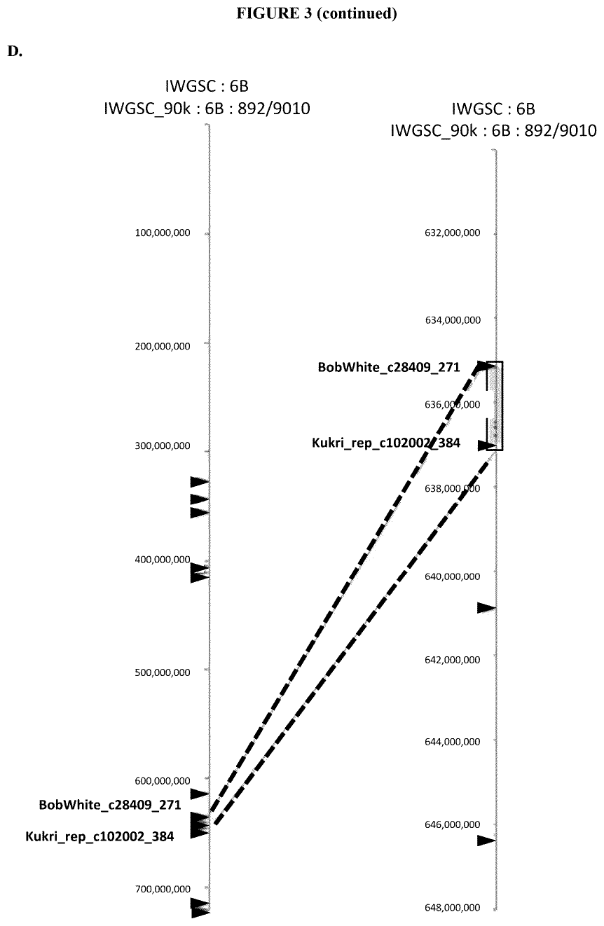

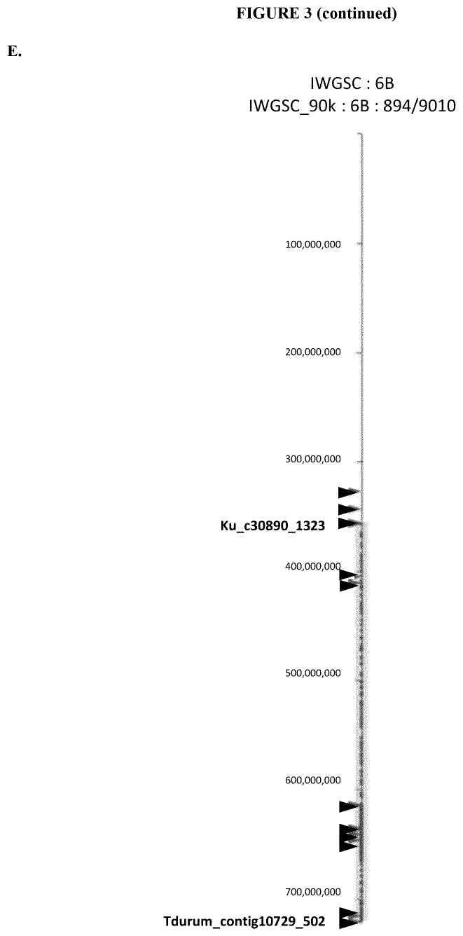

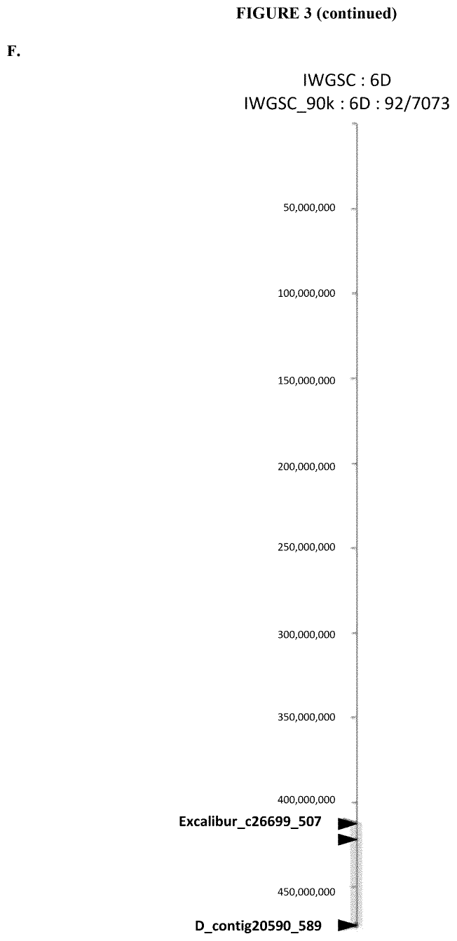

Results from the analysis are shown in FIG. 3 by way of a simple schematic representation of the chromosome being analyzed. The chromosomes referred to are 6A for A nulls (21B_710) and partial A12null (21B_674); chromosome 6D for D null (19B_34); and chromosome 6B for the Bnulls (20B_241), B1 (22A_28) & B23 (20B_602). The estimated size of the deletions on the chromosome 6A ranged from 13 million bp (A partial null) to 250 million bases (A null) (FIG. 3A). The estimated size of the deletions on the chromosome 6B for the B partial null 2 million bases up to 370 million bases in the full B null (FIG. 3B). The estimated size of the deletions on the chromosome 6D for the D null mutant was less than 100 million bp (FIG. 3C).

This analysis demonstrates that a broad range of deletions was identified in the HIB screening, with partial and full deletions of Amy1 genes for each of the three genomes. Of the lines selected for SNP analysis, six lines were selected for further analysis to evaluate the effects of a partial or complete deletion of the Amy1 locus across the A, B and D wheat genomes.

### Example 4—Generation of Amy1 Lines with Combined Genomic Mutations

In order to generate double null Amy1 mutant plants, plants of the six three wheat lines An (null for Amy1 in A genome), A12n (partial null for Amy1 cluster in A genome). Bn (null for Amy1 in the B genome), B1n (partial null for Amy1 in B genome), B23n (partial null for Amy1 in B genome) and Dn (partial null for Amy1 in D genome) were used in a series of crosses. Crossing of the single null lines resulted in the following double nulls being obtained: AnB23n, A12nBn, A12nBin, A12nB23n, B1nDn, B23nDn. Presence or absence of Amy1 genes was confirmed using the PCR method described elsewhere herein.

In order to generate triple null Amy1 mutant plants, plants of the selected double nulls were crossed and two independent triple null lines were obtained. Presence or absence of Amy1 genes was confirmed using the PCR method described in elsewhere herein. The A12nB1nDn line was generated from a cross between A12nBin and B1nDn and has 4 copies left of the Amy1 genes out of 11. The other triple null line obtained was a new A12nB23Dn generated from a cross between A12nB23n and B23nDn which has 5 copies of the Amy1 genes retained but showed a strong growth penalty.

A double null AnBn was not obtained. This inability of generating this specific double null may be the demonstration of Amy1 locus significant reduction on grain viability. As for each genome, the Amy1 locus is composed into distant cluster, it is also possible that a physiologically very important gene was deleted, thus causing viability issue.

These lines were grown at the same time under the same growing conditions, grain harvested at plant maturity, and the grain dried to about 9% moisture content (on a weight basis). These grain lots were analyzed for various parameters including seed weight, starch content, amylose content, total dietary fibre, and lipid content.

To produce plants and grain in two different genetic backgrounds other than HIB, single null mutants An, Bn and Dn were used as pollen donors in crosses to plants of the wheat cultivars Chara, Seri and Kennedy, with use of the DNA markers in each generation to detect and select the mutant alleles in the progeny. Seri and Kennedy are LMA constitutive expressers and LMA susceptible lines, thereby representing testing varieties for LMA resistance. For each generation selected single null mutant progeny were used in 3 successive back-crosses to Chara as recurrent parent, resulting in BC2 plants. Single null mutants were crossed and selfed, producing F2 progeny, from which a total single null Amy1 plants (designated as “abd” genotype) were selected. The F2 progeny included wild-type, single null Amy1 genotypes as well as all combinations of heterozygotes. Introgression of the specific Amy1 mutation on “clean” non-mutated Chara replacing approximately 82.5% of the HIB genome by a mutation free genome while retaining the Amy1 mutation was achieved using the An, Bn and Dn HIB mutants. These plants were grown at the same time under the same growing conditions, grain harvested at plant maturity, and the grain dried to about 9% moisture content (on a weight basis). These grain lots were analyzed for various parameters including seed weight, starch content, amylose content, total dietary fibre, and lipid content.

### Example 5—Grain Compositional Analysis of Single and Double Mutants

Complete grain composition was assessed from studies conducted for the single, and double null lines grown in designed and replicated single pot experiment in Glasshouse at CSIRO Black Mountain Science and Innovation Park, Canberra ACT Australia. Plants were grown in late spring, under natural light with temperatures regulated to 20° C. during the day and 14° C. at night. Plants in single pots were watered daily to maintain pot capacity.

The average grain weight (mg per grain) of the single null and double null Amy1 mutants and the wild-type grain in three genetic backgrounds was calculated by measuring the weight of 1000 grains (TGW) from each line. Average TWG ranged from 23 mg to 41 mg for Amy1 null mutants and 35 mg for the wild-type lines. When compared to the control a reduced TWG was observed for the Bn single null and A12nBn double null mutant. The A null mutant lines had a tendency to have an increased grain weight and other mutant lines showed no significant change or a tendency to have an increased grain weight (FIG. 4).

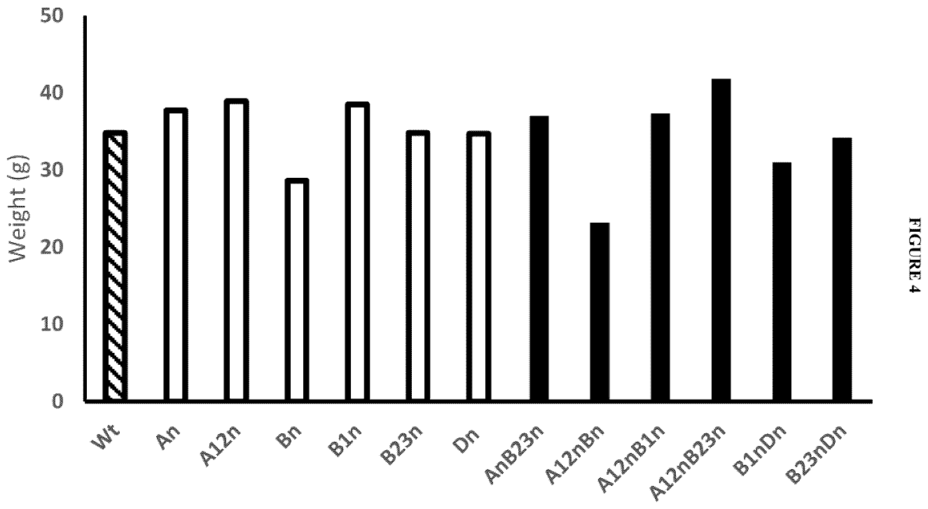

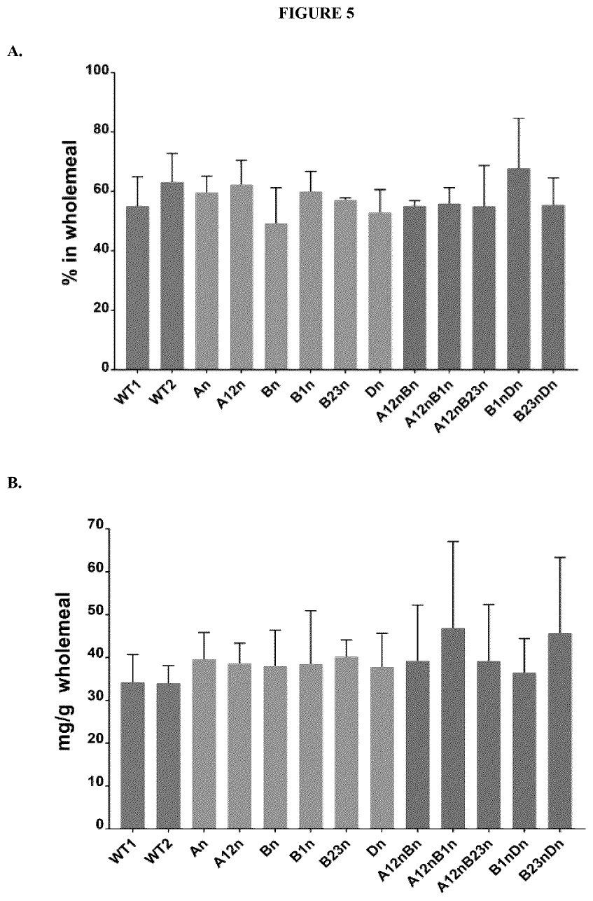

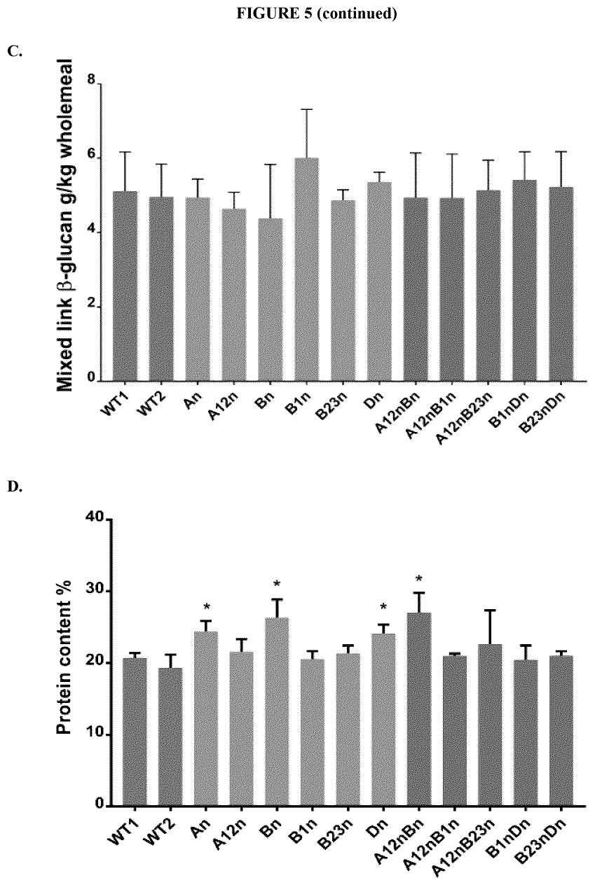

The starch content was not affected in the mutant lines. It was found that the total starch measured in wholemeal from the mutant lines was not significantly different between the wild-type, the single and the double nulls (FIG. 5A). Further analysis of the soluble sugars showed the mutant lines were similar to the wild-type controls (FIG. 5B). Similarly, no differences were found on β-glucan levels (FIG. 5C). However, analysis of total percentage nitrogen in wholemeal suggest a protein content increase for the full nulls on the A and B genome (An and Bn, respectively), as well as the partial D null (Dn) and the double mutant carrying full B null (A12nBn, FIG. 2D).

### Example 6—Starch Structural and Physiological Properties of Single and Double Null Mutants

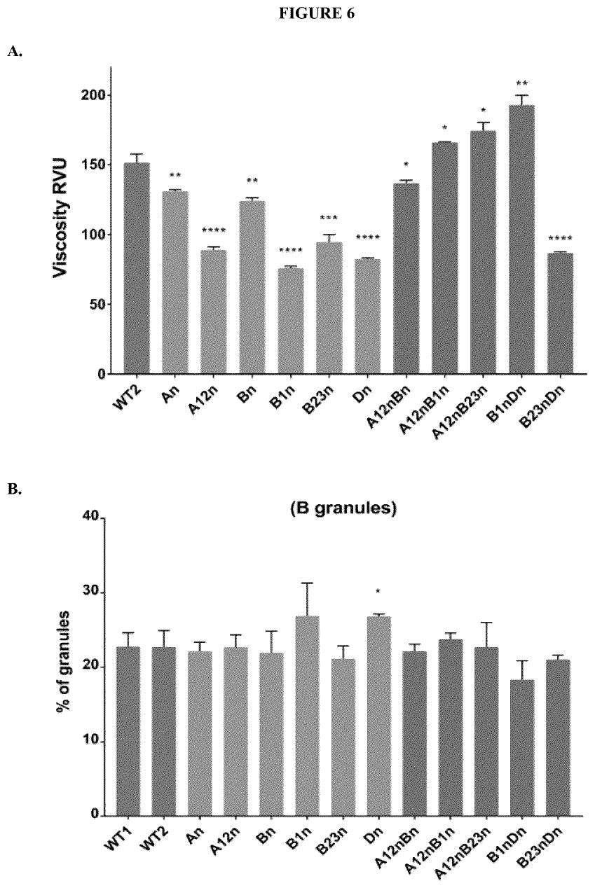

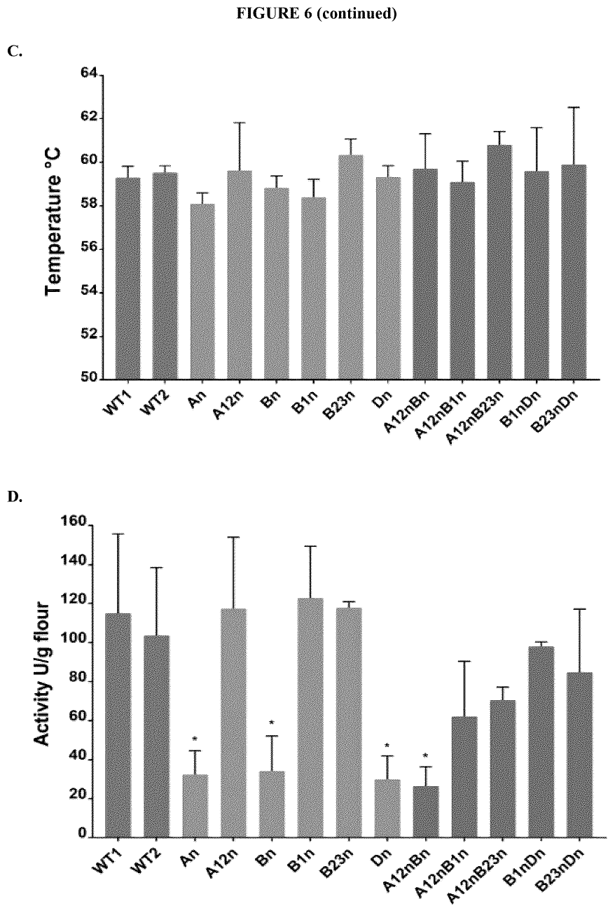

Complete starch characterization including starch granule size distribution, Rapid Visco Analysis (Viscosity) and chain length distribution were assessed for the single, and double null lines grown in designed and replicated single pot experiment in Glasshouse at CSIRO Black Mountain Science and innovation Park, Canberra ACT (FIG. 6). There was an overall decrease in peak viscosity for all the single and double null lines compared to wild-type (FIG. 6A). The decrease was more severe for full A and B nulls but the lowest viscosity was observed in the partial D null. There was no impact on starch structure (data not shown), granule size (FIG. 6B) or gelatinization properties (FIG. 6C).

As α-amylase accumulates over the first days of grain germination, total a-amylase activity was evaluated at a 72 h germination time point in the single and double null lines (FIG. 6D). Dormancy was broken down before imbibition in order to “synchronize” the physiological grain conditions. Grains were imbibed on filter paper with water, at room temperature and in the dark. Grain were sampled at appropriate time points and freeze dried before milling. α-amylases were extracted, and activity quantified using following the Megazyme Ceralpha kit. The total α-amylase activity was considerably reduced for the full A and B null, partial D null and double null with full B null. A decrease in activity was also observed for the partial double nulls but was less considerable than the single full nulls.

### Example 7—Characterization of LMA Resistance

A bulk up trial was conducted in Glasshouse at CSIRO Black Mountain Science and Innovation Park, Canberra ACT. Nitrogen input was applied to mimic field practice and aimed to boost grain production toward a potential outdoor field trial. Each line was grown on single bench and contained 20 large pots containing 6 seeds each. The experiment was replicated twice for the single mutants An, Bn and Dn. Seeds were sown at the end of April and grain was harvested in September. Due to the growing period corresponding to the autumn and winter, glasshouse conditions were suitable to trigger LMA according to Mares and Mrva (2014). Complete LMA assessment was undertaken on the grains from these growing and bulk up trials. Tests for Falling Number, total α-amylase activity, Mass spectrometry (MRM) and LMA ELISA were undertaken according to methods described elsewhere herein.

### Falling Number

Falling Number measurements were performed on wholemeal flour obtained from grain pooled and collected from the lines on each bench. Approximately 4 tests per bench were performed and compared to a standard wholemeal flour MACE from a commercial provider (not grown under LMA conditions) and to the Chara wild-type parent grown in glasshouse and under LMA conditions. The Falling Number measurements are presented in FIG. 4. While the commercial control flour showed a Falling Number of 460, the lines grown in glasshouses showed a dramatic reduction of Falling Number to the minimal value of 65-70 seconds except for the where Bn mutant was grown (FIG. 4). A12Bn could not be analyzed due to the very low yield. While An showed a falling number of around 120-150, Bn was the only line with an acceptable falling number above 250.

### Total α-Amylase Activity

Total α-amylase activity was measured using the Megazyme biochemical assay kit as described elsewhere herein. The kit can detect total α-amylase activity but does not discriminate between the different isoforms of α-amylase (i.e., type 1, type 2 or type 3). Low levels of total α-amylase activity were found in commercial control sample of the wheat cv. Mace. Most of the lines showed elevated level of total α-amylases including the parent control Chara. However, low activity levels were found in wholemeal flour from An, A12nBn and Bn with all lines carrying Bn having an α-amylase activity level comparable to the non-affected control flour (FIG. 8).

### Germination Assay

Germination time course was performed in lab environment on grain harvested at physiological maturity in accordance with the methods described in Example 1. Coleorhizae emergences were monitored every 24 hours for a week until no new emergence were observed.

As alpha-amylase accumulates over the first days of grain germination, total alpha-amylase activity in the single and double Amy1 mutant lines was assessed at the 72 h germination time point (FIG. 6D). Dormancy was broken down before imbibition in order to “synchronise” the physiological grain conditions. Grains were imbibed on filter paper with water, at room temperature and in the dark. Grains were sampled at appropriate time points and freeze dried before milling. Alpha-amylases were extracted, and activity quantified using following the Megazyme Ceralpha kit, as described elsewhere herein. The total alpha-amylase activity was reduced in the full A and B null, partial D null and double null with full B null Amy1 mutant lines. A decrease in activity was also observed for the partial double nulls, but to a lesser extent as compared to the single full nulls.

Germination rate in lines carrying An, Bn and Dn mutations remained high despite the reduction of total alpha-amylase activity from 24 to 72 hours. The line carrying the Bn mutation reached 100% emergence in 48 hours. The other mutant lines and the control Chara reached 90% emergence after 48 hours. Accordingly, while several null combinations showed a reduction in total alpha-amylase activity, null combinations comprising Amy1-6B mutations did not result in germination concerns.

### Analysis of AMY Proteins by Mass Spectrometry

Currently there is no method to detect the individual gene proteins present in the grain or flour. Falling Number does not discriminate between any starch degrading enzymes, which may results in false positives due to modification of starch structure. Total α-amylase activity similarly does not discriminate between the different families of α-amylase. The LMA ELISA test is specific to the AMY1 family but not to specific genes among the 12 genes.

Using the set of mutants from the HIB described above, a targeted mass spectrometry method was developed, employing a multiple reaction monitoring (MRM) method to detect the presence of specific peptides that map to each of the described Amy1 genes across the three genomes. This advantageously enables the identification of active Amy1 genes in the LMA phenotype. Further, this technique allows for the analysis of several peptides in a single experiment.

Using 72 hr sprouted control flour, a set of sprouting-associated peptides were detected, including 35 AMY1 related peptides and 9 AMY2 related peptides (Table 5).

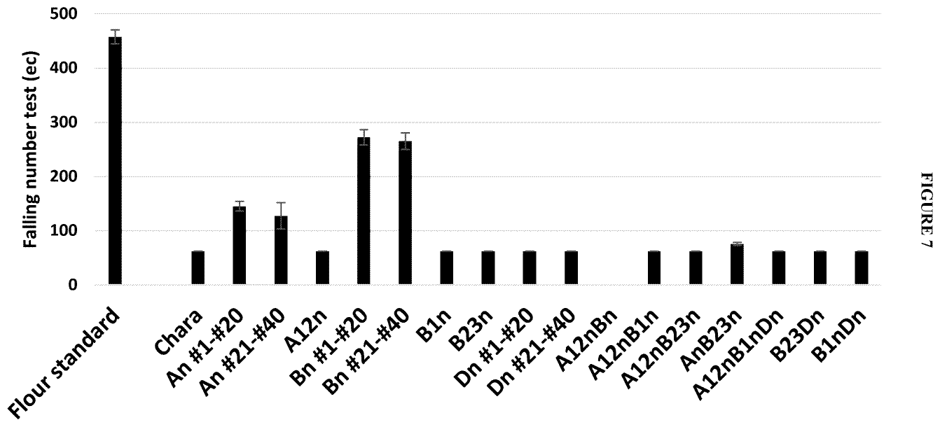

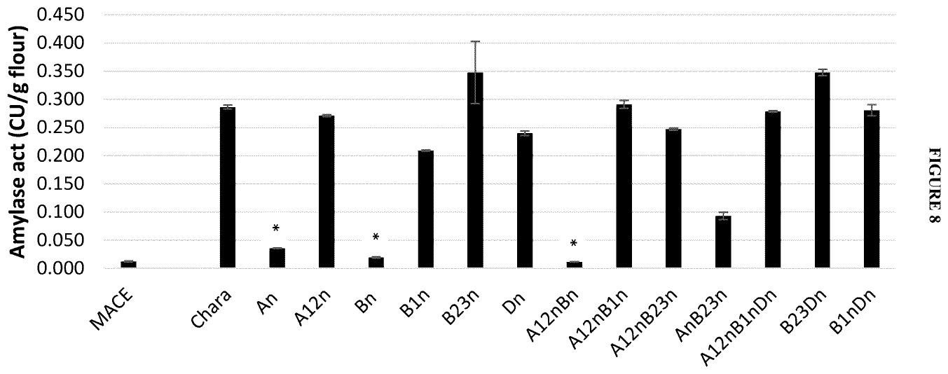

Analysis performed on the mutant lines indicated that LMA susceptible plants presented between 70 to 95% of the AMY1-related peptides, while containing less than 20% of the AMY2-related peptides. This result confirmed the LMA nature of the low Falling Number for the mutant lines, except for the triple null mutant lines with reduced susceptibility to LMA, e.g., Bn (FIG. 9).

An and Bn show a very limited presence of AMY1 peptides confirming the low alpha-amylase activity and associated higher Falling Number.

### Example 8—Detection of Reduced Susceptibility to LMA

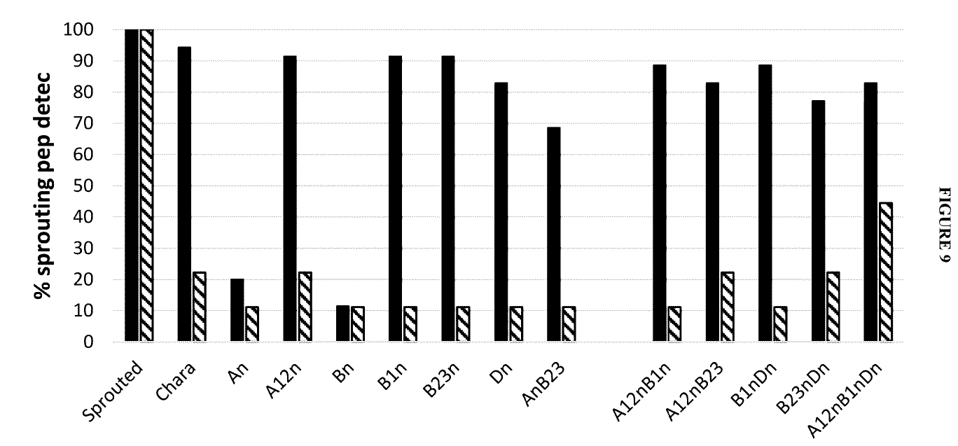

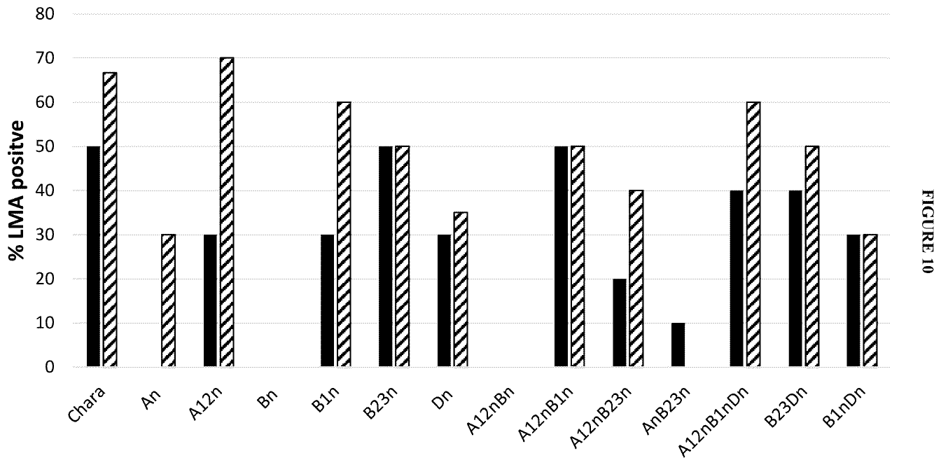

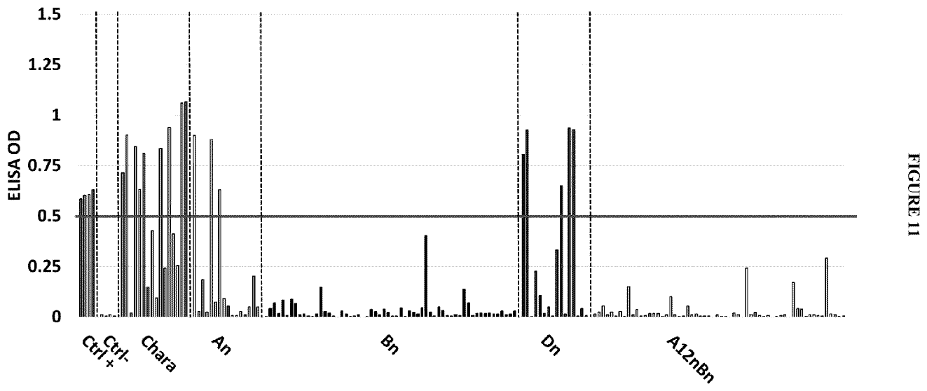

An LMA ELISA was test performed on 10 individual seeds for each mutant lines and wholemeal from all mutants including A12nBn, which confirmed the LMA nature of the lower FN (FIG. 10). When grain is sprouted, α-amylase production is triggered by embryo releasing and diffusing Gibberellic acid hormones. Therefore, the α-amylase content is much stronger on the embryonic part of the grain. However, when LMA is triggered, the α-amylase expression is randomly distributed across the grain and shows very little difference between the embryonic and non-embryonic.

The presence of AMY1 protein was strong and uniform in both the embryonic part and non-embryonic part of the grain, suggesting the presence of LMA rather than sprouting. Among the mutants tested, An and AnB23n showed a mild response which matches to the previous FN and α-amylase activity measurements. Bn and A12nBn showed a total absence of AMY1 in either the embryonic or non-embryonic part of the grain, indicating that Bn mutation may be sufficient to reduce the presence of AMY1 below the detection level when LMA is triggered.

In order to confirm the absence of positive test on Bn and A12Bn, 60 seeds were tested individually for LMA in both mutants A12nBn and Bn, and compared to An, Dn and Chara (FIG. 8). While the Chara and Dn lines showed an average of 40 to 60% LMA affected grain, the An line presented only a 30% contamination. More importantly, none of the 60 seeds tested in both Bn and A12nBn lines showed significant level of LMA ELISA response.

LMA testing was also conducted by the National LMA testing facility (Adelaide, Australia) over the 2019-2020 season. Twenty grains from each single and double null variety were anonymized and provided to testing facility for analysis.

The LMA testing facility grew 2 plans per pot and tag 4 heads in total (2 per plant). For each line, 2 or 3 pots were grown. The data indicated that an average of 6 to 10 single ELISA test (wells) were performed per lines. The ELISA results had little variation (˜3% variation between technical reps), with most of the variability coming from the greenhouse phase. The results were listed at a 0.05 and 0.01 statistical threshold (TRUE indicating lines passed).

Eighteen different controls were used by the testing facility and provided expected results (including Hartog being LMA negative and Seri, Cranbook and Kennedy being LMA sensitive). Among the 15 anonymized varieties, Chara (Background), Seri and Hartog were included and provided similar results to those from the control panel. From the AMY1 KO variety set, mutants carrying the full Bn deletion either alone or in combination with A12n were deemed LMA resistant at 0.05 and 0.01 statistical threshold respectively. A12n was reported as passing the LMA test as well but the presence of LMA in An, AnB23n, A12nB1n and A12nB23n (all carrying similar mutation to the A12n) could indicate that tested A12n grains “escaped trigger” rather than a true phenotype indicative of reduced susceptibility to LMA. All other mutants were reported as having LMA present.

The absence of LMA in both Bn and A12nBn replicates the results obtained using the MRM analysis method described elsewhere herein.

### Example 9—Field Trial Assessment of AMYKO Lines

### Growing Conditions

To assess the agronomic performance of the AMY1KO lines, or Amy1 knock out lines, in field conditions. The AMY1 KO lines refers to the single and double null mutants identified and developed by the inventors. A replicated plot trial was performed at Boroowa Experimental Research Station (Boroowa, NSW, Australia) during the 2020 growing season. Single null An, Bn and Dn had 6 replicated plots, while the rest of the AMY1KO lines were sown in triplicate.

### Results

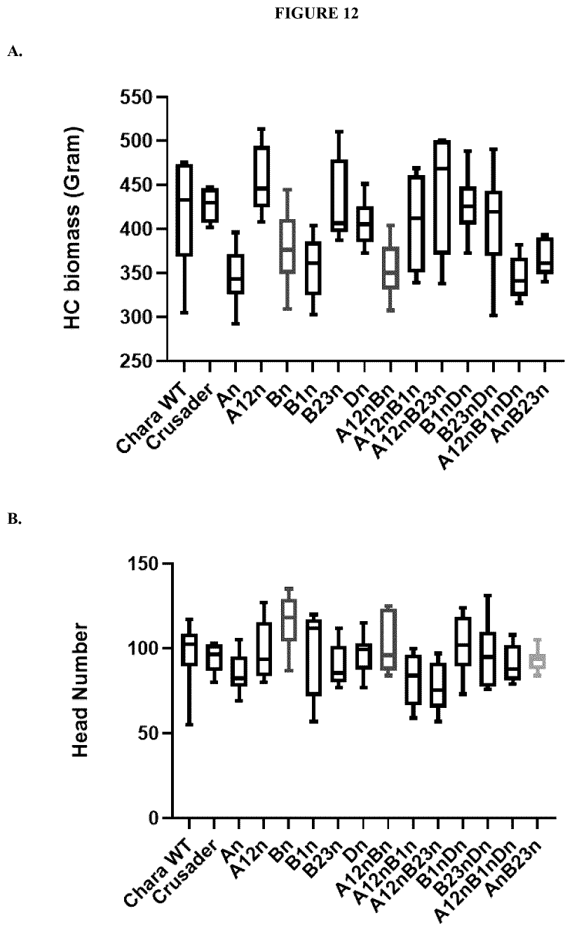

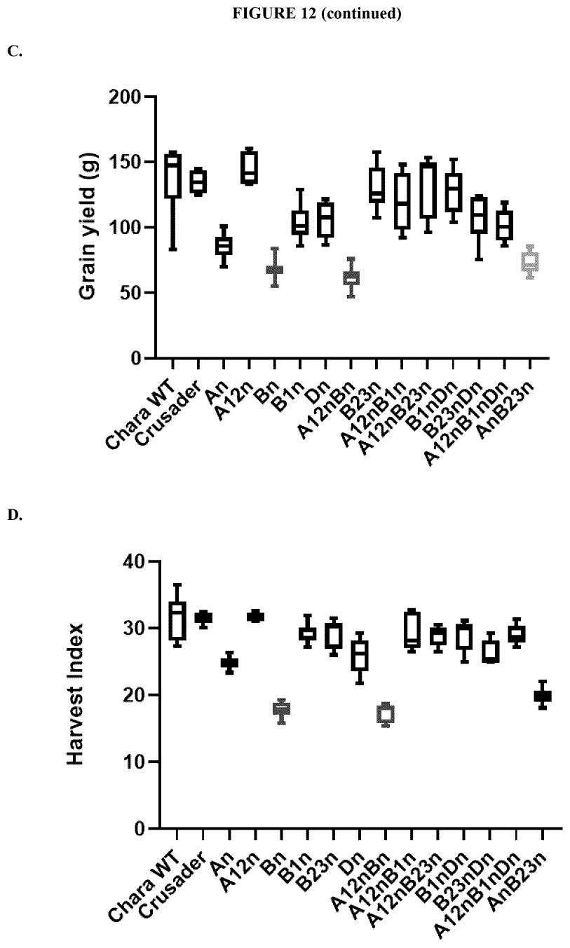

Non-significant variations in biomass were observed across the AMY1 KO population with the AMY1KO varying from 293 g to 500 g while Chara (from 305 to 475 g per Harvest cut (HC)) and Crusader (402 to 448 g per HC) remained within the same range (FIG. 12A).

Tiller numbers also remained in the same range averaging around 100 tiller per harvest cut. The tiller number from lines carrying the Bn mutation was in the higher part of the spectrum (FIG. 12B). However, grain yield was significantly reduced for some of the AMY1 KO lines most notably lines carrying the B null mutation (62 g per HC on average) and the A null mutation (86 g per HC on average) were lower than the control lines. All the mutations maintained similar yield to that displayed by Chara WT (FIG. 10C). Combination of significant reduction of grain size associated with no statistical differences in term of biomass lead to an overall significant decrease in Harvest Index for the AMY1KO lines carrying the B null mutation.

The combination of increased head number, decreased grain yield and a trend of reducing biomass in the field present only in full Bn mutant but absent in B1n and B23n mutants (the two partial deletions surrounding the Amy1-6B locus) indicate that the complete deletion of the region located between the two Amy1-6B clusters may be responsible for the yield reduction.

Strategies to overcome this yield penalty include (i) a targeted gene editing approach to inactivate all AMY1B genes; and (ii) crossing two partial 6B mutants (e.g., Bln and B23n).

### Example 10—Development of B Null Variants of AMY1KO Lines

### Crossing of Mutants

To overcome the yield penalty observed in the field assessment of Example 8 associated with the Bn lines (Bn and A12nBn), the two partial B null mutants (B1n and B23n) were crossed to generate the same LMA resistance without yield penalty. The cross between the two partial mutations of the TaAmy1B locus carrying smaller deletion allows for production of the same LMA resistance without the impediment related to a large genome deletion. Cross-pollination between B1n and B23n generated 4 F1 heterozygous plants generating 50-75 seeds per plants. A sub-sample of 35 seeds from one single plant was germinated and screening revealed the presence of 2 lines with the desired deletions B1nB23n homozygous plants. The plants were grown to maturity and the grains collected.

LMA assessment was undertaken on the B1nB23n grains from these growing trials. Tests for falling number, total α-amylase activity, MRM and LMA ELISA were undertaken according to methods described elsewhere herein.

Further glasshouse pot trials were harvested at the start of summer at CSIRO Science and Innovation Park (Canberra, ACT). The pot trials compared the homozygote double mutant carrying small deletion (i.e., B1n23n) and the large deletion Bn line to the Chara wild-type line. The purpose was to assess the impact of the deletions on grain size and yield potential. The deletions were confirmed in the F2 population by real-time PCR as described elsewhere herein. The deletions in the F2 lines were confirmed as homozygote stable.

The B1nB23n homozygous plants showed improved development when compared to the Bn plants grown under the same conditions. The B1nB23n homozygous plants exhibited development similar to that of the control Chara wild-type line. The B1nB23n homozygous plants also showed a restoration of the grain weight with a similar result to that of the control Chara wild-type line as assessed by Thousand Grain Weight (TGW). The Thousand Grain Weight (TGW) was approximately 47 mg per 1000 seeds for the B1nB23n homozygous plants, whereas the TGW for the Bn plants was approximately 16.8 mg per 1000 seeds.

### Gene Editing Mutations

In an alternative method to generate LMA resistant lines, CRISPR/Cas9 gene editing is expected to be used to inactivate all Amy1B genes. The CRISPR/Cas9 approach to mutant generation is expected to inactivate the genes on chromosome 6B without introducing other issues resulting from the large deletions that are generated by HIB, such as phenotype drag.

Initially, the gene editing approach will focus on the introduction of mutations in conserved structures of the AMY1 polypeptides to affect protein folding, structure and/or enzymatic function. For example, modification of certain residues in the active site (e.g., T231 of SEQ ID NO: 32) or the carbohydrate binding domain (e.g., Y400 of SEQ ID NO: 24). In particular, modification of T231 of SEQ ID NO: 32 may introduce AMY1-specific structural affects, as AMY1 is associated with the display of a polar neutral side chain amino acid (threonine), whereas TaAMY2 and TaAMY3 both had an acidic amino acid (aspartic acid). Further, modification of amino acids in the carbohydrate binding domain may reduce or eliminate the ability of AMY1 to bind the substrate (starch) thus reducing the ability to digest starch and lower Falling Number.

Other gene editing strategies for the introduction of a genetic modification leading to reduced expression of at least one Amy1 gene on chromosome 6B include the introduction of a deletion mutation, an insertion mutation, a substitution mutation, a splice-site mutation, a premature translation termination mutation, a nonsense mutation and a frameshift mutation which reduces the level or activity, or both, of AMY1 polypeptides.

### Discussion

LMA is a genetic and environmentally-induced grain quality defect resulting from abnormally high levels of alpha-amylase. LMA is a relatively recently identified quality issue that is now receiving increasing attention worldwide and whose prevalence is now seen as impeding the development of superior quality wheat varieties.

The approach taken by the present inventors was to identify the Amy1 gene(s) that are responsible for some of the key characteristics of LMA, including low Falling Number, and elevated levels and/or activity of AMY1 polypeptides. As shown elsewhere herein, reduction in the expression of at least one Amy1 gene on chromosome 6B is sufficient to reduce the amount or activity of AMY1 polypeptides, which can mitigate the deleterious effects caused by elevated levels and/or activity of AMY1 polypeptides (i.e., reduced susceptibility to LMA). Accordingly, the introduction of a genetic modification leading to reduced expression of at least one Amy1 gene on chromosome 6B advantageously provides a means to control the downstream release of AMY1 polypeptides, which are triggered via complex mechanisms associated with LMA. Importantly, the genetic modification of at least one Amy1 gene on chromosome 6B do not result in any consistent deleterious impact on starch structure or other important properties of the wheat grain. Further, agronomic performance (e.g., yield, germination rate) were not adversely affected by the genetic modifications contemplated herein.

It has therefore been surprisingly demonstrated that of the complex Amy1 locus, the Amy1 genes on chromosome 6B (i.e., 5 of 12 genes) can be targeted for genetic modification to reduce susceptibility to LMA. In particular, by targeting the Amy1 genes on chromosome 6B, the expression or activity of AMY1 polypeptides is sufficiently controlled to reduce the accumulation of AMY1 polypeptides throughout the grain (i.e., embryonic, non-embryonic tissue, aleurone), thereby resulting in an acceptable Falling Number (i.e., greater than 200 seconds), and/or reducing the level or activity of alpha-amylase such that deleterious effects associated with LMA are ameliorated or avoided completely.

TABLE 1
Field trial characteristics

| | |
|---|---|
| Location | Boroowa (NSW) |
| Sowing density | 130 seeds/m² |
| (seeds per m²) | |
| Plot size (m²) | 5.2 m long × 1.8 m wide (10 rows × 18 cm |
| | spacing) = 9.36 m² |
| | Harvest dimensions 4.6 m long × 1.44 m |
| | wide (inside 8 rows) = 6.62 m² |
| Nitrogen | 100 kg/ha CROPLIFT ®15 (14.6% N) |
| application | (sowing) |
| (kg · Ha⁻¹) | 150 kg/Ha urea 6 Aug. 2020 |
| | 100 kg/Ha urea 18 Sep. 2020 |
| Planting date | 15 May 2020 |
| Emergence | First to second week of June 2020 |
| Anthesis | Early lines started approximately 5 months |
| | after planting on the 12 Oct. 2020 |
| | Later lines started approximately 8-10 days |
| | after the early lines |

Harvest

| | |
|---|---|
| Hand harvest date | 14 Dec. 2020 |
| Machine harvest date | 13 Jan. 2021 |

TABLE 2
Primers for mutation analysis

| | | | SEQ | |
|---|---|---|---|---|
| Tar- | Direc- | | ID | |
| get | tion | Name | NO: | Sequence |
| A | Forward | TaAmy1_ | 45 | CTCAACTGGCTCAGGAC |
| | | A1_F1 | | CAA |
| A | Reverse | TaAmy1_ | 46 | CCATTGTGCTTCCACGA |
| | | genABD_R1 | | CTC |
| B1 | Forward | Amy1_ | 47 | GTAGGTGCCGCGATCAT |
| | | B1I1_F1 | | GTA |
| B2 | Forward | Amy1_ | 48 | AGCTCCTATACGTACCG |
| | | Bmix50I1_F1 | | CGA |
| D | Forward | Amy1_D1I1_ | 49 | AAGATATTGTCCGCCGG |
| | | F1 | | CAA |
| ABD | Reverse | Amy1_ABDI1_ | 50 | ACTCCCAGTTGAAACCC |
| | | R | | TGC |

TABLE 3
Mutant lines analyzed by 90K SNP Chip

| Line | Deletion(s) detected | Reference |
|---|---|---|
| An | All AMY1 genes, 3 in total, deleted from | 21B_710 |
| | genome A. | |
| A12n | Two AMY1 genes deleted, 1 copy | 21B_674 |
| | remaining - TaAMY1- A3. | |
| Bn | All AMY1 genes, 5 in total, deleted from | 20B_241 |
| | genome B. | |
| B1n | Three AMY1 genes deleted, 2 copies | 22A_28 |
| | remaining - TaAMY1- B1, TaAMY1- | |
| | B2. | |
| B23n | Two AMY1 genes deleted. 3 copies | 20B_602 |
| | remaining - TaAMY1-B3, TaAMY1- | |
| | B4, TaAMY1-B5. | |
| Dn | Two AMY1 genes deleted, 1 copy | 19B_34 |
| | remaining - TaAMY1-D | |

TABLE 4
Minimum Falling Number required for Australian wheat grades

| Wheat Grade | Minimum Falling Number (seconds) |
|---|---|
| APH2 | 350 |
| H1, H2, APW1, APW1, ASW1, | 300 |
| AUH2, HSP1 | 250 |
| AGP1 | 200 |

TABLE 5
Sprouting peptides for determining
susceptibility to LMA

| | | SEQ |
|---|---|---|
| Sprouting | | ID |
| Peptide | Peptide Sequence | NO: |
| AMY1-A1 | QVLFQGFNWESWK | 59 |
| | SEPSFAVAEIWTSLAYGGDGKPNLNQDQHR | 60 |
| | AVTFVDNHDTGSTQHMWPFPSDK | 61 |
| AMY1-A2 | VDDIAAAGVTHVWLPPASQSVAEQGYMPGR | 62 |
| | LYDLDASK | 63 |
| | SLIGALHGK | 64 |
| | AIADIVINHR | 65 |
| | GIYCIFEGGTPDAR | 66 |
| | DDRPYADGTGNPDTGADFGAAPDIDHLNPR | 67 |
| | ELVEWLNWLR | 68 |
| | GYSADVAK | 69 |
| | QELVNWVNK | 70 |
| | VGGSGPGTTFDFTTK | 71 |
| | GILNVAVEGELWR | 72 |
| | APGMIGWWPAK | 73 |
| | AVTFVDNHDTGSTQHMWPFPSDR | 74 |
| | LQIIEADADLYLAEIDGK | 75 |
| | YDVGHLIPQGFK | 76 |
| | VVAHGNDYAVWEK | 77 |
| AMY1-A3 | VGAHGNDYAVWEK | 78 |
| AMY1-B2 | VDDIAAAGVTHVWLPPASQSVSEQGYMPGR | 79 |
| | TDIGFDGWR | 80 |
| AMY1-B5 | VDDIAAAGITHVWLPPASQSVAEQGYMPGR | 81 |
| AMY1-D1 | VDDIAAAGVTHVWLPPASQSVSK | 82 |
| | SEASFAVAEIWTSLAYGGDGKPNLNQDPHR | 83 |
| | FDVGHLIPGGFK | 84 |
| | DYAVWEK | 85 |
| AMY1-D3 | AVTFVDNHDTGSTQHMWPFPSDR | 86 |
| | ELVEWLNWLK | 87 |
| | ADIGFDGWR | 88 |
| | QELVNWVDK | 89 |
| | GPATTFDFTTK | 90 |
| | LQIIEADSDLYLAEIDGK | 91 |
| | YDVGHLIPGGFK | 92 |
| | VAAHGNDYAVWEK | 93 |
| AMY2-A2 | GVQAIADIVINHR | 94 |
| | VGGAASAGMVFDFTTK | 95 |
| AMY2-B1 | GIYCIFEGGTSDGR | 96 |
| | SDLGFDAWR | 97 |
| | GYSPEMAK | 98 |
| | QNLVNWVDK | 99 |
| | GILNAAVEGELWR | 100 |
| | APGVMGWWPAK | 101 |
| | ILMHEGDAYVAEIDGK | 102 |
| | AVTFVDNHDTGSTQALWPFPSDK | 103 |

1. A wheat plant comprising a genetic modification leading to reduced expression of at least one Amy1 gene on chromosome 6B relative to a wild-type wheat plant, wherein grain from the plant is characterized by a reduced level or activity, or both, of alpha-amylase 1 (AMY1) polypeptides relative to a wild-type wheat grain.

2. The wheat plant of claim 1, wherein grain from the plant is further characterized by one or more, or all, of the features selected from:
a. a Falling Number of at least 200 seconds;
b. reduced total alpha-amylase activity relative to a control level;
c. reduced expression of AMY1 polypeptides or peptides in the grain relative to a control level; and
d. has reduced susceptibility to late-maturity alpha-amylase (LMA) relative to wild-type wheat grain.

3. The wheat plant of claim 1 or claim 2, wherein the at least one Amy1 gene on chromosome 6B is selected from Amy1-6B-1, Amy1-6B-2, Amy1-6B-3, Amy1-6B-4 and Amy1-6B-S.

4. The wheat plant of any one of claims 1 to 3, which has reduced expression of at least two, at least three, or at least four Amy1 genes on chromosome 6B.

5. The wheat plant of claim 3, which has reduced expression of all five Amy1 genes on chromosome 6B.

6. The wheat plant of any one of claims 1 to 5, wherein expression of the at least one Amy1 gene is reduced to an undetectable level.

7. The wheat plant of any one of claims 1 to 6, wherein the genetic modification produces the reduced expression of at least one Amy1 gene on chromosome 6B via gene silencing.

8. The wheat plant of any one of claims 1 to 6, wherein the genetic modification is a mutation selected from a deletion mutation, an insertion mutation, a substitution mutation, a splice-site mutation, a premature translation termination mutation, a nonsense mutation and a frameshift mutation.

9. The wheat plant of claim 8, wherein the mutation is a deletion mutation, which deletes all or a part of at least one Amy1 gene on chromosome 6B.

10. The wheat plant of claim 8, wherein the deletion mutation deletes all or part of a nucleic sequence of SEQ ID NO: 13, 17, 21, 25 and/or 29, or a nucleic acid sequence which is at least 90% identical to the sequence of SEQ ID NO: 13, 17, 21, 25 and/or 29.

11. The wheat plant of claim 9 or claim 10, wherein the deletion mutation is a deletion of about 1-30 base pairs.

12. The wheat plant of any one of claims 1 to 11, wherein the control level is total alpha-amylase activity or expression of AMY1 polypeptides in grain from a wild-type wheat plant grown under the same conditions.

13. The wheat plant of claim 12, wherein total alpha-amylase activity is from about 1% to about 50% of the total alpha-amylase activity of grain from a wild-type wheat plant grown under the same conditions.

14. The wheat plant of claim 13, wherein the expression of AMY1 polypeptides in the grain is reduced by at least 50% relative to grain from a wild-type wheat plant grown under the same conditions.

15. The wheat plant of any one of claims 1 to 14, wherein grain from the plant is further characterized by one or more, or all, of the features selected from:
a. a content of protein that is increased relative to wild-type wheat grain;
b. a proportion of total starch that is the same as, or reduced relative to wild-type wheat grain;
c. a content of soluble sugars that is the same as, or increased relative to wild-type wheat grain;
d. starch with reduced viscosity (peak viscosity, pasting temperature, etc.) relative to wild-type wheat grain; and
e. a content of beta-glucan that is the same as, or reduced, relative to wild-type wheat grain.

16. The wheat plant of any one of claims 1 to 15, which is male and female fertile.

17. A process for producing wheat grain, comprising:
a. growing a wheat plant of any one of claims 1 to 16, preferably in a field as part of a population of at least 1000 such plant or in an area of at least 1 hectare planted at a standard planting density; and
b. harvesting grain from the plant.

18. A process for producing bins of wheat grain, comprising:
a. reaping above-ground parts of wheat plants of any one of claims 1 to 16;
b. threshing and/or winnowing the parts of the wheat plants to separate the grain from the remainder of the plant parts;
c. sifting and/or sorting the grain separated in step (b); and
d. loading the sifted and/or sorted grains into bins, thereby producing bins of grain.

19. Wheat grain obtained from the wheat plant of any one of claims 1 to 16, or produced by the process of claim 17 or claim 18.

20. Wheat grain comprising a genetic modification leading to reduced expression of at least one Amy1 gene on chromosome 6B relative to a wild-type wheat grain, wherein the grain is characterized by a reduced level or activity, or both, of AMY1 polypeptides relative to a wild-type wheat grain.

21. The wheat grain of claim 20, further characterized by one or more, or all, of the features selected from:
a. a Falling Number of at least 200 seconds;
b. reduced total alpha-amylase activity relative to a control level;
c. reduced expression of AMY1 polypeptides or peptides in the grain relative to a control level; and
d. has reduced susceptibility to the deleterious effects of LMA relative to a wild-type wheat grain.

22. The wheat grain of claim 20 or claim 21, which is further characterized by a feature of as defined in any one of claims 3 to 15.

23. The wheat grain of any one of claims 20 to 22, which has been processed so that it is no longer capable of germinating, such as kibbled, cracked, par-boiled, rolled, pearled, milled or ground grain.

24. The wheat grain of any one of claims 20 to 22, which is capable of growing into a wheat plant when the grain is sown into soil.

25. A wheat cell derived from the wheat plant of any one of claims 1 to 16 or the wheat grain of any one of claims 20 to 24.

26. A nucleic acid molecule which encodes an AMY1 polypeptide, wherein the nucleic acid molecule differs from the nucleic acid of SEQ ID NO: 13, 17, 21, 25 and/or 29, or a nucleic acid sequence which is at least about 91% identical to the nucleic acid sequence of SEQ ID NO: 13, 17, 21, 25 and/or 29, by a mutation selected from a deletion mutation, an insertion mutation, a splice-site mutation, a premature translation termination mutation, a nonsense mutation and a frameshift mutation.

27. The nucleic acid molecule of claim 26, wherein the mutation is a deletion mutation, which deletes all or a part of the sequence set forth as SEQ ID NO: 13, 17, 21, 25 and/or 29, or a nucleic acid sequence which is at least about 91% identical to the nucleic acid sequence of SEQ ID NO: 13, 17, 21, 25 and/or 29.

28. The nucleic acid molecule of claim 27, wherein the deletion is about 1-30 base pairs.

29. The nucleic acid molecule of any one of claims 26 to 28, which is in a cell, preferably in a wheat cell, or in a food product.

30. An AMY1 polypeptide, wherein the polypeptide differs from the amino acid sequence of SEQ ID NO: 16, 20, 24, 28 and/or 32, or an amino acid sequence which is at least 99% identical to the amino acid sequence of SEQ ID NO: 16, 20, 24, 28 and/or 32, by at least one amino acid substitution, insertion or deletion in the active site, catalytic site, calcium binding site and/or carbohydrate binding domain, wherein the AMY1 polypeptide has an impaired structure and/or reduced activity relative to an AMY1 polypeptide having the amino acid sequence of SEQ ID NO: 16, 20, 24, 28 and/or 32.

31. The AMY1 polypeptide of claim 30, wherein the polypeptide differs from the amino acid sequence of SEQ ID NO: 32 by at least one amino acid substitution, insertion or deletion in:
a. the active site, wherein the at least one amino acid substitution, insertion or deletion is between amino acid 33 and 312 of SEQ ID NO: 32;
b. the catalytic site, wherein the at least one amino acid substitution, insertion or deletion is between amino acid 203 and 313 of SEQ ID NO: 32; and/or
c. the calcium binding site, wherein the at least one amino acid substitution, insertion or deletion is between amino acid 115 and 172 of SEQ ID NO: 32.

32. The AMY1 polypeptide of claim 30 or claim 31, in which the at least one amino acid substitution, insertion or deletion is selected from:
a. at least one amino acid substitution, insertion or deletion of an amino acid corresponding to amino acid W33, Y75, H116, F167, R201, F204, K206, W230, T231 or H312 of SEQ ID NO: 32;
b. at least one amino acid substitution, insertion or deletion of an amino acid corresponding to amino acid D203, E228 or D313 of SEQ ID NO: 32; and/or
c. at least one amino acid substitution, insertion or deletion of an amino acid corresponding to amino acid N115 or D172 of SEQ ID NO: 32.

33. The AMY1 polypeptide of claim 30, wherein the at least one amino acid substitution, insertion or deletion is between amino acid 294 and 418 of SEQ ID NO: 24.

34. The AMY1 polypeptide of claim 33, wherein the at least one amino acid substitution, insertion or deletion corresponds to amino acid Y400 of SEQ ID NO: 24.

35. A method of producing wheat flour, wholemeal, starch, starch granules or bran, the method comprising obtaining the grain of any one of claims 19 to 23 and processing the grain to produce the flour, wholemeal, starch, starch granules or bran.

36. Wheat flour, wholemeal, starch, starch granules or bran produced by the method of claim 35, or comprising the nucleic acid molecule of any one of claims 26 to 29 or the polypeptide of any one of claims 30 to 34.

37. A method of producing a food product, comprising mixing the grain of any one of claims 19 to 23, or the wheat flour, wholemeal, starch, starch granules or bran of claim 36 with at least one other food ingredient to produce the food product.

38. A food product comprising the wheat grain of any one of claims 19 to 23, the wheat cell of claim 25, the nucleic acid molecule of any one of claims 26 to 29, the polypeptide of any one of claims 30 to 34, or an ingredient which is the wheat flour, wholemeal, starch, starch granules or bran of claim 36.

39. The food product of claim 38, which is leavened or unleavened bread, pasta, noodle, breakfast cereal, snack food, cake, pastry or a flour-based sauce.

40. A method of genotyping a wheat plant or grain, the method comprising:
a. obtaining a sample comprising nucleic acid extracted from a wheat plant or grain; and
b. detecting in the sample a nucleic acid molecule of any one of claims 26 to 29.

41. A method of selecting a wheat plant from a population of wheat plants, wherein said population of plants comprises progeny plants obtained from a cross between two plants of which at least one plant was a wheat plant of any one of claims 1 to 16, the method comprising:
a. genotyping each one or more progeny plants in said population of wheat plants using the method of claim 40; and
b. based on the results of the genotyping in step (a), selecting a progeny plant which comprises the nucleic acid molecule of any one of claims 26 to 29.

42. A method for reducing susceptibility to LMA in a wheat plant, the method comprising:
a. crossing a first parent wheat plant with a second parent wheat plant, wherein the second plant is a wheat plant of any one of claims 1 to 16; and
b. backcrossing a progeny plant of the cross of step (a) with a plant of the same genotype as the first parent plant to produce a plant with a majority of genotype of the first parent but comprising a nucleic acid molecule of any one of claims 26 to 29.

43. The method of claim 42, wherein the progeny is genotyped for the presence or absence of the nucleic acid molecule using the method of claim 40.

44. A method of producing a plant with reduced susceptibility to LMA, the method comprising:
a. introducing a genetic modification to at least one Amy1 gene on chromosome 6B to a plant cell such that the cell has reduced level or activity, or both, of AMY1 polypeptides relative to unmodified cells;
b. regenerating a plant with the genetic modification from the cell of step (a), wherein the plant has reduced susceptibility to LMA relative to wild-type plants lacking the genetic modification.

45. The method of claim 44, further comprising harvesting the seed from the plant.

46. The method of claim 44 or claim 45, further comprising crossing the plant with the genetic modification with a second plant to produce one or more progeny plants.

47. A method of determining susceptibility to LMA in a wheat plant, the method comprising:
a. obtaining a sample of grain from the wheat plant;
b. analyzing the sample for the presence of at least one AMY1 peptide selected from the amino acid sequences of SEQ ID NOs: 59-93, or an amino acid sequence which comprises not more than three modifications to an amino acid sequence of SEQ ID NOs: 59-93; and/or at least one AMY2 peptide selected from the amino acid sequences of SEQ ID NOs: 94-103, or an amino acid sequence which comprises not more than three modifications to an amino acid sequence of SEQ ID NOs: 94-103;
c. comparing the peptides analyzed in step (b) to those present in control wheat grain, and based on this comparison, determining if the wheat plant is susceptible to LMA.

48. The method of claim 47, wherein the control wheat grain is a sprouted wheat grain.

49. The method of claim 47 or claim 48, wherein the sample of grain is milled or ground grain.

50. The method of claim 47 or claim 48, wherein the sample of grain is an extract of the grain.

51. The method of any one of claims 47 to 50, wherein the AMY1 and/or AMY2 peptides are analyzed by targeted mass spectrometry.

52. The method of claim 51, wherein the targeted mass spectrometry comprises a multiple reaction monitoring (MRM) method.

53. The method of any one of claims 47 to 53, wherein the AMY1 peptides comprising the amino acid sequences of SEQ ID NOs: 59-93 and the AMY2 peptides comprising the amino acid sequences of SEQ ID NOs: 94-103 are analyzed in step (b).

54. The method of claim 53, wherein the wheat plant is determined to be susceptible to LMA when:
a. at least about 50% of the AMY1 peptides comprising the amino acid sequences of SEQ ID NOs: 59-93 are present in the sample relative to the control wheat grain; and
b. less than about 50% of the AMY2 peptides comprising the amino acid sequence of SEQ ID NOs: 94-103 are present in the in the sample relative to the control wheat grain.

55. The method of claim 54, wherein from about 50% to about 100% of the AMY1 peptides comprising the amino acid sequences of SEQ ID NOs: 59-93 are present in the sample.

56. The method of claim 54 or claim 55, wherein from about 0% to about 50% of the AMY2 peptides comprising the amino acid sequences of SEQ ID NOs: 94-103 are present in the sample.

57. The method of claim 53, wherein the wheat plant is determined to have reduced susceptibility to LMA when:
a. less than about 30% of the AMY1 peptides comprising the amino acid sequences of SEQ ID NOs: 59-93 are present in the sample relative to the control wheat grain; and
b. less than about 30% of the AMY2 peptides comprising the amino acid sequence of SEQ ID NOs: 94-103 are present in the in the sample relative to the control wheat grain.
